(19) United States
(12) Patent Application Publication
(10) Pub. No.: US 2026/0337208 A1
(43) Pub. Date: Oct. 29, 2026

(54) HETEROCYCLIC DERIVATIVES AS JANUS KINASE INHIBITORS

(71) Applicant: CHIESI FARMACEUTICI S.P.A., Parma (IT)

(72) Inventors: Alessandro ACCETTA, Parma (IT); Alberto CUZZOLIN, Parma (IT); Fabio RANCATI, Parma (IT); Andrea RIZZI, Parma (IT); Ivaylo Jivkov ELENKOV, Parma (IT); Milan MESIC, Parma (IT); Claudio FIORELLI, Parma (IT)

(73) Assignee: CHIESI FARMACEUTICI S.P.A., Parma (IT)

(21) Appl. No.: 19/109,284

(22) PCT Filed: Sep. 8, 2023
(86) PCT No.: PCT/EP2023/074692

§ 371 Date: Mar. 6, 2025

(30) Foreign Application Priority Data
Sep. 9, 2022 (EP) 22194841.7

(51) Int. Cl. C07D 519/00; A61K 31/519; A61K 31/538; A61K 31/5415; A61K 45/06; C07D 471/04

(52) U.S. Cl. CPC C07D 519/00; A61K 31/519; A61K 31/538; A61K 31/5415; A61K 45/06; C07D 471/04

(57) ABSTRACT

Provided is a compound of formula (I) inhibiting the JAK family of non-receptor tyrosine protein kinases (JAK1, JAK2, JAK3, and TYK2). Methods of preparing such compounds, pharmaceutical compositions containing the compounds, and therapeutic use of the compounds are also described. In particular, the compound can be used to treat diseases or conditions associated with dysregulation of the JAK family non-receptor kinases, for example, for treating various inflammatory diseases, including asthma, COPD, and other respiratory diseases.

### FIELD OF THE INVENTION

The present invention relates to chemical compounds that are derivatives useful as JAK inhibitors, such as JAK 1, useful for the treatment of various inflammatory disease including asthma, COPD and other respiratory diseases.

### BACKGROUND OF THE INVENTION

The JAK family consists of non-receptor tyrosine protein kinases and has four main members, JAK1, JAK2, JAK3, and TYK2. More than 50 cytokines and growth factors bind to type I and II receptors noncovalently associated with different combinations of JAK kinases. The signalling triggered by the ligands consists in tyrosine phosphorylation of receptors by JAK and recruitment of one or more STATs proteins. Tyrosine-phosphorylated STATs dimerize and are then transported into the nucleus through the nuclear membrane to regulate specific genes. JAKs have seven homology domains (the JAK homology domain, JH). Starting from the carboxyl terminus, JH1 is the first JH, known as the kinase domain, and is composed of approximately 250 amino acid residues. JH1 encodes a kinase protein that constitutes the kinase structure domain that phosphorylates a substrate; JH2 is a pseudokinase domain which regulates the activity of the kinase domain. JAK3 is expressed in the bone marrow and lymphatic system, as well as endothelial cells and vascular smooth muscle cells; other members are expressed in almost all tissues (Hu X et al., Signal Transduct Target Ther. 2021, 26; 6 (1): 402). Many cellular processes are downstream JAK/STAT signalling: hematopoiesis, immune balance, tissue repair, inflammation, apoptosis, and adipogenesis. Different biological responses are regulated by specific pairing of JAK isoforms. JAK1/JAK3 combination mediates IL-2, -4, -7, -9, -15, and -21 signalling that is relevant for growth/maturation of lymphoid cells, differentiation/homeostasis of T-cells/NK cells, B-cell class switching and other inflammatory processes. Combinations of JAK1/TYK2-JAK1/JAK2, regulate the signal associated with the innate immune response, such as IL-6 and the type I interferons, involved into naïve T cell differentiation, T cell homeostasis, granulopoiesis and other inflammatory processes. (Howell M D et al., Front. Immunol. 2019, 10, 2342). JAK2 frequently associates with itself (JAK2/JAK2) controlling the signalling of various cytokines and growth factors, such as IL-3, IL-5, granulocyte macrophage colony-stimulating factor (GM-CSF), erythropoietin (EPO), and thrombopoietin (TPO) (Hodge et al., Clin Exp Rheumatol 2016; 34(2): 318-28).

Genetically modified mouse models and human diseases prove the importance of JAK/STAT pathways in immune fitness. In particular, overexpression or mutations involving some JAK isoforms as well as aberrant JAK/STAT signalling drive malignancies of hematopoietic and lymphoid tissues as well as inflammatory disorders. Currently, several Food and Drug Administration (FDA)- and/or EU-approved JAK inhibitors are in clinical use. Two (ruxolitinib and fedratinib) small molecules are in use for hematologic disorders as myelofibrosis and polycythemia vera; six JAK inhibitors (tofacitinib, baricitinib, ruxololitinib, filgotinib, upadicitinib and delgocitinib in Japan) result in use for immune-mediated disorders as rheumatoid arthritis, polyarticular juvenile idiopathic arthritis, atopic dermatitis, ulcerative colitis and acute graft-versus-host disease. Moreover, some of these drugs as well as others are currently under phase II and III of clinical trials for indications that span from autoimmune diseases (lupus, vitiligo, etc.), inflammatory bowel disease to Non-Hodgkin lymphoma and COVID-19 (Hu X. et al., Sig Transduct Target Ther 2021, 6:402).

The small molecules targeting JAK/STAT represent an attractive option also for the therapy of fibrotic disorders. In fact, inflammatory cytokines (IL-4, IL-3, IL-6, IL-11, IL-31, etc) and growth factors (FGF, VEGF, etc.) involved in the fibrotic processes activate JAK/STAT pathway. Ruxolitinib tested in a bleomycin-induced fibrosis mouse model ameliorated the fibrotic lesions in lung, and reduced levels of fibrotic molecular markers (Zhang, Y et al., Ann. Rheum. Dis. 2017, 76, 1467-1475) while tofacitinib acted as a preventive agent in experimental dermal and pulmonary fibrosis (Wang, W et al., Scleroderma Relat. Disord. 2020, 5, 40-50). In patients, some case reports were studied. A single-6 case report corroborated the efficacy and safety of tofacitinib in combination with nintedanib in the management of an aggressive interstitial lung disease with poor prognosis (Conca, W et al., Front. Pharmacol. 2020, 11, 5857619). Baricitinib was demonstrated to be a safe immune modulator that reduces the biomarkers' levels of lung fibrosis and inflammation in RA patients, including a subgroup with interstitial lung disease (D'Alessandro M et al., Int. Immunopharmacol. 2020, 86, 106748).

In COVID-19, there are some JAK inhibitors undergoing clinical trials, and they are tofacitinib, baricitinib, and ruxolitinib. Baricitinib and ruxolitinib were associated with a reduced risk of mortality. They reduced the use of invasive mechanical ventilation and had a borderline impact on the admission rate of the intensive care unit and the incidence of acute respiratory distress syndrome (ARDS). (Wijaya, I. et al. Clin. Epidemiol. Glob. Health 2021, 11, 100755). Ruxolitinib also was tested in COVID-19 patients, and improved the clinical symptoms and chest computed tomography images (Cao Y. et al., J. Allergy Clin. Immunol. 2020 146, 137-146).

Asthma can be included in the plethora of immune-mediated diseases for which pathogenesis is characterized by an essential role of JAK/STAT signalling. Asthma is a chronic inflammatory disease of the airways due to a complex interplay between immune response, genetic susceptibility and nonspecific external stimuli like cold, allergens and exercise leading to hyperresponsiveness, remodelling of the airways, ultimately contributing to airflow limitation. Severe asthma affects 5% to 15% of the population with adult asthma (which is 300 million people worldwide) and represents a public health issue associated with increased mortality, increased hospitalizations, significant burden of symptoms, health care costs, and missed work and school (Steve N G et al., J Allergy Clin Immunol 2021; 148:953-63). Severe asthma represents a subset of difficult-to-treat asthma and occurs in patients whose disease remains uncontrolled despite the use of high doses of inhaled corticosteroids (ICSs) combined with long-acting β-agonists or other controllers. To date, four types of biologics are licensed for severe asthma, i.e. omalizumab (anti-immunoglobulin E) antibody, mepolizumab and reslizumab (anti-interleukin [IL]-5antibody), benralizumab (anti-IL-5 receptor a antibody) and dupilumab (anti-IL-4 receptor alpha antibody). Despite their efficacy, many patients continue to experience exacerbations or uncontrolled disease, indicating a need for more novel therapies (Israel E, Reddel H K. N Engl J Med 2017; 377:965-76).

Recently, the better understanding of asthma pathobiology brought to a shift from a phenotypic classification system to the introduction of the “endotype” concept. According to the latter, classification is performed on the basis of pathophysiologic mechanisms and clinical biomarkers associated with a given patient (Wenzel S E et a., Am J Respir Crit Care Med 2021; 203:809-21). There are two major endotypes in asthma: type 2 and non-type 2. The type 2 pathway is defined by activation of cytokines derived from TH2 cells and group 2 innate lymphoid cells (ILC2s); these include IL-4, IL-5, and IL-13 that cause airway inflammation by activating eosinophils, B cells, airway epithelial cells, and other cell types. Biomarkers of type 2 asthma include blood/sputum eosinophilia and elevated levels of fractional exhaled nitric oxide (FENO) and IgE. The type 2-low pathway is characterized by absence of type 2-high cytokines and biomarkers, and it manifests either increased levels of neutrophils in the airways or a paucigranulocytic profile, with normal levels of airway neutrophils and eosinophils. Type 2-low asthma is currently not well understood, and it likely encompasses multiple distinct endotypes. Potential mediators and/or biomarkers of T2 low endotypes under investigation include IL-6, IL-17A/F, IL-23, Type I interferons, CXCL10, TNF, alarmins (TSLP, IL-25, IL-33), IL-1β, IL-8, IFN-γ (Hinks T S C et al., ERJ 2021, 57 (1) 2000528).

Almost all the mediators mentioned above both for T2 and T2-low endotypes activate JAK/STAT pathway, here the rationale for the potential use of JAK inhibitors in both endotypes of severe asthma. Targeting simultaneously several cytokines by JAK inhibitors may offer advantage over the biologics (for no-responder patients) and standard therapies (for patients who remain uncontrolled) considering their administration on top of ICS.

Despite strong rationale of JAK inhibitors in asthma, safety concerns may arise by administration of systemic inhibitors or may limits administration into particular asthma subjects such as children. Considering that Asthma is a lung restricted disease, inhalatory route of administration for a JAK inhibitor may offers the advantage of therapeutic efficacy while limiting systemic exposure and correlated side effects. To date, some companies are developing inhaled JAK inhibitor for asthma treatment. Astrazeneca pipeline include AZD-0449 (completed Phase I clinical trial) and AZD-4604 (ongoing Phase I clinical trial); Theravance Biopharma is starting a new preclinical program on TD-8236 inhaled JAK inhibitor and Kinaset/Vectura is developing VR588 (ongoing Phase I clinical trial) as inhalatory compound. Many preclinical studies sponsored by the companies mentioned above demonstrated the efficacy of JAK inhibitors in the modulation of asthma. In the preclinical phase of drug development, JAK1/3 inhibitor R256 (now referred as AZD0449) orally given showed be effective in decreasing airway resistance, BAL eosinophilia, mucus production and if administered during sensitization, also TH2 cytokine responses (Ashino S et al., J Allergy Clin Immunol 2014; 133:1162-74). iJak-381 from Genentech given as dry powder reduced BAL eosinophilia, CCL11, airway resistance, and Muc5AC in OVA-challenged mice. Moreover, it reduced BAL eosinophilia, neutrophilia, CCL11, and CXCL1 in a in mouse model of chronic exposure to AAH allergens (Dengler H S et al., Sci Transl Med 2018; 10: eaao2151). Moreover, an oral JAK inhibitor as Tofacitinib, formulated for being administered as aerosol, reduced eosinophils count in a house dust mite mouse model of asthma (Younis U S et al., AAPS PharmSci-Tech 2019; 20:167).

Another respiratory disease that could benefit from lung restricted JAK inhibition is Chronic obstructive pulmonary disease (COPD), an inflammatory disease of the lung, most commonly resulting from cigarette smoke exposure, characterised by a largely irreversible and progressive airflow limitation. Despite inflammatory cytokines are drivers of chronic airway inflammation and some of them trigger JAK/STAT activation (IL-6, IFN-γ, IL-2, etc.), the role of this pathway in COPD pathogenesis is poorly characterized. Phosphorylated-STAT4+ cells (Di Stefano A et al., Eur Respir J. 2004 July; 24 (1): 78-85) were found to be increased in COPD compared to non-smokers healthy controls. In another study, phosphorylated-STAT3+ and phosphorylated-STAT1+ cells counts were higher in lung biopsies of COPD patients than non-smokers controls while it was not possible to reproduce previous data on phosphorylated-STAT4 molecule (Yew-Booth L et al., Eur Respir J 2015; 46 (3): 843-5). These data might also suggest a therapeutic use of JAK inhibitors also in COPD disease.

Despite inhalatory administration, safety concerns may still arise by drug levels reaching systemic circulation following inhalation of a JAKi. In addition to a well suited profile for inhalation, a JAKi should preferably possess additional properties that may further limit the systemic exposure after inhalation.

Strong need remains for JAK inhibitors, and particularly inhaled JAK inhibitors that have potential for giving compounds with an improved safety.

In view of the number of pathological responses which are mediated by JAK enzymes, there is a continuing need for inhibitors of JAK enzymes which can be useful in the treatment of many disorders and particularly respiratory diseases.

Thus, the finding of novel safe and potent JAK inhibitor suitable for local administration to the lungs for treatment of asthma and respiratory disease still remains an important need.

### SUMMARY OF THE INVENTION

Accordingly, it is one object of the present invention to provide compounds of formula (I)

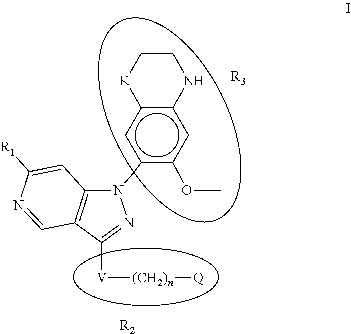

Wherein n, K, V and Q and R₁ are as defined in the detailed description of the invention; or a pharmaceutically acceptable salt thereof, that are useful as JAK kinase inhibitors.

It is another object of the present invention to provide pharmaceutical compositions comprising such compounds, methods of using such compounds to treat respiratory diseases, and processes and intermediates useful for preparing such compounds.

In one aspect, the present invention provides a compound of formula (I) for use as a medicament. In one aspect the present invention provides the use of a compound of the invention for the manufacture of a medicament.

In a further aspect, the present invention provides the use of a compound of the invention for the preparation of a medicament for the treatment of any disease associated with JAK enzyme mechanisms.

In another aspect, the present invention provides a method for prevention and/or treatment of any disease associated with JAK enzyme mechanisms as above defined, said method comprises administering to a patient in need of such treatment a therapeutically effective amount of a compound of the invention.

In a particular aspect the compounds of the invention are used alone or combined with other active ingredients and may be administered for the prevention and/or treatment of a pulmonary disease including asthma, Chronic obstructive pulmonary disease (COPD), cystic fibrosis (CF), interstitial lung diseases and idiopathic pulmonary fibrosis (IPF), acute lung injury and acute respiratory distress syndrome (ARDS).

### DETAILED DESCRIPTION OF THE INVENTION

### Definitions

The term “Pharmaceutically acceptable salts” refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts of the invention comprise ions of alkali or alkaline earth metals such as potassium, sodium, calcium or magnesium. Those obtained by reacting the main compound, functioning as a base, with an inorganic or organic acid to form a salt comprise, for example, salts of hydrochloric, hydrobromic, sulfuric, phosphoric, methane sulfonic, camphor sulfonic, acetic, oxalic, maleic, fumaric, succinic and citric acids.

Many organic compounds can form complexes with solvents in which they are reacted or from which they are precipitated or crystallized. These complexes are known as “solvates” which are a further object of the invention. Polymorphs and crystalline forms of compounds of formula (I), or of pharmaceutically acceptable salts, or solvates thereof are a further object of the invention.

The term “Halogen” or “Halo-” or “halogen atoms” includes fluorine, chlorine, bromine, and iodine atom; meaning Fluoro, Chloro, Bromo, Iodo as substituent.

The term “(C₁-C₆)Alkyl” refers to straight-chained or branched alkyl groups wherein the number of carbon atoms is in the range 1 to 6. Particular alkyl groups are for example methyl, ethyl, n-propyl, isopropyl, t-butyl, 3-methylbutyl and the like.

The expressions “(C₁-C₆)Haloalkyl” refer to the above defined “(C₁-C₆)alkyl” groups wherein one or more hydrogen atoms are replaced by one or more halogen atoms, which can be the from other. Examples include halogenated, same or different each poly-halogenated and fully halogenated alkyl groups wherein all of the hydrogen atoms are replaced by halogen atoms, e.g. trifluoromethyl or difluoro methyl groups.

By way of analogy, the terms “(C₁-Cₓ) hydroxyalkyl” or “(C₁-Cₓ) aminoalkyl” refer to the above defined “(C₁-Cₓ) alkyl” groups wherein one or more hydrogen atoms are replaced by one or more hydroxy (OH) or amino group respectively, and wherein x is an integer up to 10. Thus, “(C₁-C₆)hydroxyalkyl” or “(C₁-C₆)aminoalkyl” refers to said hydroxy- or amino-alkyl groups wherein the number of carbon atoms is in the range 1 to 6.

The definition of aminoalkyl encompasses alkyl groups (i.e. “(C₁-C₆)alkyl” groups) substituted by one or more amino groups (—NR₄R₅). An example of aminoalkyl is a mono-aminoalkyl group such as R₄R₅N—(C₁-C₆)alkyl, or —(CH₂)ₘNR₄R₅. Wherein R₄ and R₅ and m are as defined in the detailed description of the invention.

With reference to the substituent R₄ and R₅ as above defined, it is here further explained that when either R₄ and R₅ are taken together with the nitrogen atom they are linked to form a 5 to 6 membered heterocyclic radical, at least one further ring carbon atom in the said heterocyclic radical may be replaced by at least one heteroatom or hetero-group (e.g. N, NH, S or O) or may bear an -oxo (═O) substituent group. The said heterocyclic radical might be further optionally substituted on the available points in the ring, namely on a carbon atom, or on an heteroatom or hetero-group available for substitution. Thus, Examples of said heterocycle radicals are 1-pyrrolidinyl, 1-piperidinyl, 1-piperazinyl, 4-morpholinyl, piperazin-4yl-2-one, 4-methylpiperazine-1-yl.

The term “(C₃-C₁₀)cycloalkyl” likewise “(C₃-C₆)cycloalkyl” refers to saturated cyclic hydrocarbon groups containing the indicated number of ring carbon atoms. Examples include cyclopropyl, cyclobutyl, cyclopentyl, cyclohexyl and cycloheptyl, and polycyclic ring systems such as adamantan-yl.

The expression “Aryl” refers to mono, bi- or tri-cyclic carbon ring systems which have 6 to 20, preferably from 6 to 15 ring atoms, wherein at least one ring is aromatic. The expression “heteroaryl” refers to mono-, bi- or tri-cyclic ring systems with 5 to 20, preferably from 5 to 15 ring atoms, in which at least one ring is aromatic and in which at least one ring atom is a heteroatom (e.g. N, S or O).

Examples of aryl or heteroaryl monocyclic ring systems include, for instance, phenyl, thienyl, pyrrolyl, pyrazolyl, imidazolyl, isoxazolyl, oxazolyl, isothiazolyl, thiazolyl, pyridinyl, pyrimidinyl, pyrazinyl, pyridazinyl, triazinyl, furanyl radicals and the like.

Examples of aryl or heteroaryl bicyclic ring systems include naphthalenyl, biphenylenyl, purinyl, pteridinyl, pyrazolopyrimidinyl, benzotriazolyl, benzoimidazole-yl, quinolinyl, isoquinolinyl, indolyl, isoindolyl, indazolyl, benzothiopheneyl, benzodioxinyl, dihydrobenzodioxinyl, indenyl, dihydro-indenyl, dihydrobenzo[1,4]dioxinyl, benzothiazole-2-yl, dihydrobenzodioxepinyl, benzooxazinyl, 1,2,3,4-tetrahydroisoquinoline-6-yl, 4,5,6,7-tetrahydrothiazolo[4,5-c]pyridine, 4,5,6,7-tetrahydrobenzo[d]thiazol-2-yl, 5,6,7,8-tetrahydro-1,7-naphthyridine, radicals and the like.

Examples of aryl or heteroaryl tricyclic ring systems include fluorenyl radicals as well as benzocondensed derivatives of the aforementioned heteroaryl bicyclic ring systems.

The derived expression “(C₃-C₁₀)heterocycloalkyl” likewise “(C₃-C₆)heterocycloalkyl” refers to saturated or partially unsaturated mono, bi- or tri-cycloalkyl groups of the indicated number of carbons, in which at least one ring carbon atom is replaced by at least one heteroatom (e.g. N, NH, S or O) and/or may bear an -oxo (═O) substituent group (e.g. C(═O), S(═O)₂). Said heterocycloalkyl (i.e. heterocyclic radical or group) is further optionally substituted on the available points in the ring, namely on a carbon atom, or on an heteroatom available for substitution.

Substitution on a carbon atom includes spiro disubstitution as well as substitution on two adjacent carbon atoms, in both cases thus form additional condensed 5 to 6 membered heterocyclic ring. Examples of (C₃-C₆)heterocycloalkyl are represented by: oxetanyl, tetrahydro-furanyl, pyrrolidinyl, imidazolidinyl, thiazolidinyl, piperazinyl, piperidinyl, morpholinyl, thiomorpholinyl, dihydro- or tetrahydro-pyridinyl, tetrahydropyranyl, pyranyl, 2H- or 4H-pyranyl, dihydro- or tetrahydrofuranyl, dihydroisoxazolyl, pyrrolidin-2-one-yl, dihydropyrrolyl, 5-oxopyrrolidin-3-yl, (1R,5S,6r)-3-oxabicyclo[3.1.0]hexan-6-yl, 1,1-dioxidothiomorpholino, octahydrocyclopenta[c]pyrrol-5-yl, 4,5,6,7-tetrahydropyrazolo[1,5-a]pyrazin-2-yl; 4,5,6,7-tetrahydrothiazolo[5,4-c]pyridine-2-yl radicals and the like.

Examples of said heterocycle radicals are 1-methyl-2-pyrrolidinyl, 1-piperidinyl, 1-piperazinyl, 4-morpholinyl, piperazin-4-yl-2-one, 4-methylpiperazine-1-yl, 1-methylpiperidin-4-yl, 4-metylpiperazine-1-yl-2-one, 7-methyl-2,7-diazaspiro[3.5]nonan-2-yl, 2-methyl-2,9-diazaspiro[5.5]undecan-9-yl, 9-methyl-3,9-diazaspiro[5.5]undecan-3-yl, and (3aR,6aS)-5-methyl-octahydropyrrolo[3,4-c]pyrrol-2-yl.

The term “Aryl(C₁-C₆)alkyl” refers to an aryl ring linked to a straight-chained or branched alkyl group wherein the number of constituent carbon atoms is in the range from 1 to 6, e.g. phenylmethyl (i.e. benzyl), phenylethyl or phenylpropyl.

Likewise the term “Heteroaryl(C₁-C₆)alkyl” refers to an heteroaryl ring linked to a straight-chained or branched alkyl group wherein the number of constituent carbon atoms is in the range from 1 to 6, e.g. furanylmethyl.

The term “alkanoyl”, refers to HC(O)— or to alkylcarbonyl groups (e.g. (C₁-C₆)alkylC(O)—) wherein the group “alkyl” has the meaning above defined. Examples include formyl, acetyl, propanoyl, butanoyl.

The term “(C₁-C₁₀) alkoxy” or “(C₁-C₁₀) alkoxyl”, likewise “(C₁-C₆)alkoxy” or “(C₁-C₆) alkoxyl” etc., refers to a straight or branched hydrocarbon of the indicated number of carbons, linked to the rest of the molecule through an oxygen bridge. “(C₁-C₆)Alkylthio” refers to the above hydrocarbon linked through a sulfur bridge.

The derived expression “(C₁-C₆)haloalkoxy” or “(C₁-C₆)haloalkoxyl” refers to the above defined haloalkyl, linked through an oxygen bridge. Example of (C₁-C₆)haloalkoxy is difluoromethoxy, trifluoromethoxy.

Likewise derived expression “(C₃-C₆)heterocycloalkyl-(C₁-C₆)alkyl” and “(C₃-C₆)cycloalkyl-(C₁-C₆)alkyl” refer to the above defined heterocycloalkyl and cycloalkyl groups linked to the rest of the molecule via an alkyl group of the indicated number of carbons, for example piperidin-4-yl-methyl, cyclohexylethyl.

The derived expression “(C₁-C₆)alkoxy (C₁-C₆)alkyl” refers to the above defined alkoxy group linked to the rest of the molecule via an alkyl group of the indicated number of carbons, for example methoxymethyl.

Likewise “(C₁-C₆)haloalkoxy(C₁-C₆)alkyl” refers to the above defined (C₁-C₆)haloalkoxy” group linked to the rest of the molecule via an alkyl group of the indicated number of carbons, for example difluoromethoxypropyl.

Likewise “(C₁-C₆)alkoxycarbonyl” refers to the above defined alkoxy group linked to the rest of the molecule via an carbonyl group.

“(C₁-C₆)alkylthiocarbonyl-” refers to the above alkylthio group linked to the rest of the molecule via a carbonyl group (C═O).

“(C₁-C₆)alkoxycarbonyl-(C₁-C₆)alkyl” refers to the above defined alkoxy group linked to the rest of the molecule via an carbonyl group further enchained with an alkyl group of the indicated number of carbons, for example methoxycarbonylmethyl.

“(C₁-C₆)alkoxycarbonyl-(C₁-C₆)alkylthio” consequently refer to the indicated enchained groups like methoxycarbonylmethylthio.

Other derived expression will be apparent in their meaning
e.g. “halo-((C₁-C₆)alkyl(C₃-C₈)heterocycloalkyl)” refer to enchained groups like 4-fluoro-1-methylpyrrolidin-3-yl.

An oxo moiety is represented by (O) as an alternative to the other common representation, e.g. (═O). Thus, in terms of general formula, the carbonyl group is herein preferably represented as —C(O)— as an alternative to the other common representations such as —CO—, —(CO)— or —C(═O)—. In general the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

When a numerical index the statement (value) “p is zero” or “p is 0” means that the substituent or group bearing the index p (e.g. Ip) is absent, that is to say no substituent, other than H when needed, is present. Likewise when the index is attached to a bridging divalent group (e.g. (CH₂)m) the statement “m in each occurrence is zero . . . ” or “m is 0” means that the bridging group is absent, that is to say it is a bond.

Whenever basic amino or quaternary ammonium groups are present in the compounds of formula (I), physiological acceptable anions, selected among chloride, bromide, iodide, trifluoroacetate, formate, sulfate, phosphate, methanesulfonate, nitrate, maleate, acetate, citrate, fumarate, tartrate, oxalate, succinate, benzoate, p-toluenesulfonate, pamoate and naphthalene disulfonate may be present. Likewise, in the presence of acidic groups such as COOH groups, corresponding physiological cation salts may be present as well, for instance including alkaline or alkaline earth metal ions.

Compounds of formula (I) when they contain one or more stereogenic center, may exist as optical stereoisomers.

Where the compounds of the invention have at least one stereogenic center, they may accordingly exist as enantiomers. Where the compounds of the invention possess two or more stereogenic centers, they may additionally exist as diastereoisomers. It is to be understood that all such single enantiomers, diastereoisomers and mixtures thereof in any proportion are encompassed within the scope of the present invention. The absolute configuration (R) or(S) for carbon bearing a stereogenic center is assigned on the basis of Cahn-Ingold-Prelog nomenclature rules based on groups' priorities.

“Single stereoisomer”, “single diastereoisomer” or “single enantiomer”, when reported near the chemical name of a compound indicate that the isomer was isolated as single diastereoisomer or enantiomer (e.g. via chiral chromatography) but the absolute configuration at the relevant stereogenic center was not determined/assigned.

Atropisomers result from hindered rotation about single bonds where the steric strain barrier to rotation is high enough to allow for the isolation of the conformers (Bringmann G et al, Angew. Chemie Int. Ed. 44 (34), 5384-5427, 2005. doi: 10.1002/anie.200462661).

Oki defined atropisomers as conformers that interconvert with a half-life of more than 1000 seconds at a given temperature (Oki M, Topics in Stereochemistry 14, 1-82, 1983).

Atropisomers differ from other chiral compounds in that in many cases they can be equilibrated thermally whereas in the other forms of chirality isomerization is usually only possible chemically.

Separation of atropisomers is possible by chiral resolution methods such as selective crystallization. In an atropo-enantioselective or atroposelective synthesis one atropisomer is formed at the expense of the other. Atroposelective synthesis may be carried out by use of chiral auxiliaries like a Corey Bakshi Shibata (CBS) catalyst, an asymmetric catalyst derived from proline, or by approaches based on thermodynamic equilibration when an isomerization reaction favors one atropisomer over the other.

Racemic forms of compounds of formula (I) as well as the individual atropisomers (substantially free of its corresponding enantiomer) and stereoisomer-enriched atropisomer mixtures are included in the scope of the present invention.

The invention further concerns the corresponding deuterated derivatives of compounds of formula (I). In the context of the present invention, deuterated derivative means that at least one position occupied by a hydrogen atom is occupied by deuterium in an amount above its natural abundance. Preferably, the percent of deuterium at that position is at least 90%, more preferably at least 95%, even more preferably 99%.

All preferred groups or embodiments described above and here below for compounds of formula (I) may be combined among each other and apply as well mutatis mutandis.

As above mentioned, the present invention provides compounds of general formula (I), acting as JAK inhibitors, to processes for the preparation thereof, pharmaceutical compositions comprising them either alone or in combination with one or more active ingredient, in admixture with one or more pharmaceutically acceptable carriers.

In a first aspect the present invention provides compounds of formula (I)

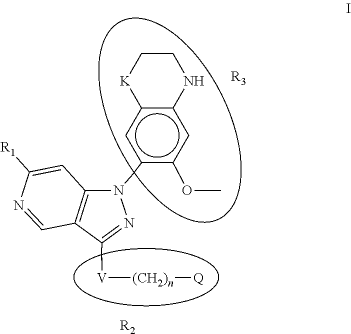

wherein
R₁ is selected from pyrazolo[1,5-a]pyrimidin-3-yl and (3-oxo-3,4-dihydropyrazin-2-yl)amino;
   graphically represented respectively by compounds of formula I-1 or I-2

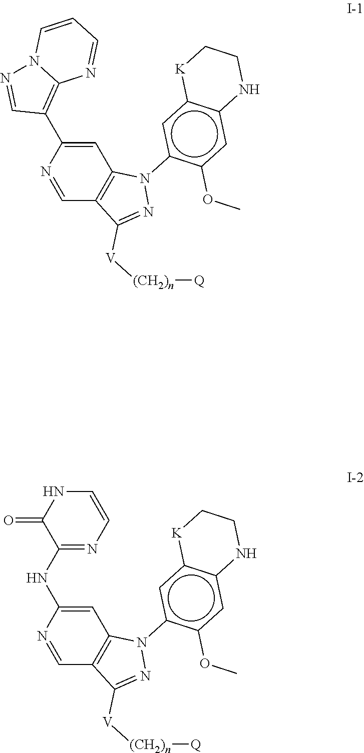

wherein the substituent group

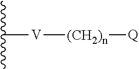

is comprising at least one ester or thioester moiety (i.e. by selecting appropriate combination of V, Q, R₄ and R₅);
V is a divalent group selected from C(O)O, C(O)N(R₆), N(R₆)C(O)O;
Q is selected from the group consisting of (C₁-C₆)alkoxycarbonyl, —(CH₂)ₘNR₄R₅, (C₃-C₈)cycloalkyl and (C₃-C₆)heterocycloalkyl; wherein said (C₃-C₈)cycloalkyl and (C₃-C₆)heterocycloalkyl are optionally substituted by one or more substituent group selected from the group consisting of (C₁-C₆)alkoxycarbonyl(CH₂)ₘ, that is preferably (C₁-C₆)alkoxycarbonyl (when m is zero), (C₃-C₈)cycloalkyl-oxycarbonyl, NC—(C₁-C₆)alkoxycarbonyl, (C₁-C₆)haloalkyl-oxycarbonyl, (C₃-C₈)heterocycloalkyl-oxycarbonyl, (C₁-C₆)alkyl(C₃-C₈)heterocycloalkyl-oxycarbonyl, (C₃-C₈)heterocycloalkyl-(C₁-C₆)alkoxycarbonyl, (C₁-C₆)alkyl-(C₃-C₈)heterocycloalkyl-(C₁-C₆)alkoxycarbonyl, (C₁-C₆)hydroxyalkyl-oxycarbonyl, (C₁-C₆)aminoalkyl-oxycarbonyl, (C₁-C₆)alkoxy-(C₁-C₆)alkyl-oxycarbonyl, (C₁-C₆)alkylthiocarbonyl, NC—(C₃-C₈)cycloalkyl(C₁-C₆)alkoxycarbonyl, NC—(C₃-C₈)cycloalkyl-oxycarbonyl, (C₁-C₆)haloalkyl-(C₃-C₈)heterocycloalkyl-oxycarbonyl, halo-((C₁-C₆)alkyl(C₃-C₈)heterocycloalkyl)-oxycarbonyl, (C₁-C₆)haloalkyl-(C₃-C₈)heterocycloalkyl-(C₁-C₆)alkoxycarbonyl, (C₁-C₆)haloalkyl-(C₃-C₈)heterocycloalkyl-oxycarbonyl-(C₁-C₆)alkyl, (C₁-C₆)hydroxyalkyl-oxycarbonyl-(C₁-C₆)alkyl, (C₁-C₁₀)alkyl and halogen;
K is selected from O, CH₂, S;
n and m are in each occurrence independently 0 or an integer selected from 1, 2, 3 and 4;
R₄ and R₅, the same or different, are selected from the group consisting of
—H,
(C₁-C₆)alkyl,
(C₁-C₆)haloalkyl,
(C₃-C₆)heterocycloalkyl
(C₁-C₆)alkyl-(C₃-C₈)heterocycloalkyl;
(C₁-C₆)alkoxycarbonyl-methyl;
(C₁-C₆)alkoxycarbonyl-phenyl-methyl; and
(C₁-C₆)alkoxycarbonyl-(C₁-C₆)alkyl-phenyl-methyl;
R₆ is in each occurrence independently selected from the group consisting of H, (C₁-C₆)alkyl, (C₁-C₆)hydroxyalkyl;
or a pharmaceutically acceptable salt or solvate thereof.

Preferred, in the above group are compounds of formula I-1 wherein K is O;
represented by the formula (Ia)

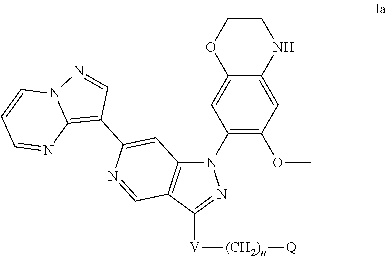

wherein
V is C(O)N(R6),
Q is (C₃-C₆)heterocycloalkyl, preferably a piperidine moiety, substituted by a group selected from (C₁-C₆)alkoxycarbonyl, (C₃-C₈)cycloalkyl-oxycarbonyl, NC—(C₁-C₆)alkoxycarbonyl, (C₁-C₆)haloalkyl-oxycarbonyl, (C₁-C₆)alkyl(C₃-C₈)heterocycloalkyl-oxycarbonyl, (C₁-C₆)hydroxyalkyl-oxycarbonyl, (C₁-C₆)alkoxy (C₁-C₆)alkyl-oxycarbonyl and (C₁-C₆)alkylthiocarbonyl;
n is in each occurrence independently 0 or an integer selected from 1, 2, 3 and 4;
R₆ is in each occurrence independently selected from the group consisting of H, (C₁-C₆)alkyl, (C₁-C₆) hydroxyalkyl;
or a pharmaceutically acceptable salt or solvate thereof.

Particularly preferred compounds in this embodiment are:

| Example No | Chemical Name |
|---|---|
| 9 | ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 45 | isopropyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 11 | cyclopropyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 25 | 2-cyano-2-methylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 13 | 2,2,2-trifluoroethyl 1-(2-(1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridine-3- |
| | carboxamido)ethyl)piperidine-4-carboxylate |
| 21 | 1-methylazetidin-3-yl 1-(2-(1-(6-methoxy-3,4-dihydro-2H-benzo[b][1,4]oxazin- |
| | 7-yl)-6-(pyrazolo[1,5-a]pyrimidin-3-yl)-1H-pyrazolo[4,3-c]pyridine-3- |
| | carboxamido)ethyl)piperidine-4-carboxylate |
| 15 | 3-hydroxy-2,2-dimethylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 14 | 2-hydroxy-2-methylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 5 | 1-methoxy-2-methylpropan-2-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 23 | (R)-1-methylpyrrolidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 59 | S-Ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carbothioate |

A further preferred embodiment in the above group of compounds of formula (I) is that wherein
V is a divalent group C(O)O;
Q is selected from the group consisting of (C₁-C₆)alkoxycarbonyl, —(CH₂)ₘNR₄R₅, and (C₃-C₆)heterocycloalkyl optionally substituted by one or more group selected from (C₁-C₁₀)alkyl and halogen;
n and m are in each occurrence independently 0 or an integer selected from 1, 2, 3 and 4;
R₄ and R₅, the same or different, are selected from the group consisting of
—H,
(C₁-C₆)alkyl,
single enantiomers, diastereoisomers and mixtures thereof
or a pharmaceutically acceptable salt or solvate thereof.

Another preferred embodiment in the above group of compounds of formula (I), when K is equal to S, is a compound represented by formula (Ib)

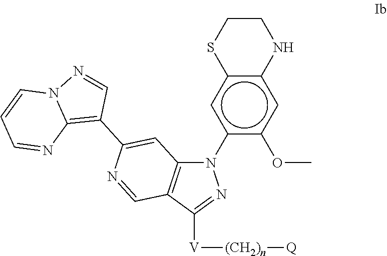

wherein
V is a divalent group selected from N(R₆)C(O)O;
Q is selected from the group consisting of (C₁-C₆)alkoxycarbonyl;
n is an integer selected from 1, 2, 3 and 4;
R6 is in each occurrence independently selected from the group consisting of H, (C₁-C₆)alkyl;
single enantiomers, diastereoisomers and mixtures thereof
or a pharmaceutically acceptable salt or solvate thereof.

Another preferred embodiment in the above group of compounds of formula (I-2), when K is equal to O or S, is a compound represented by formula (Ic)

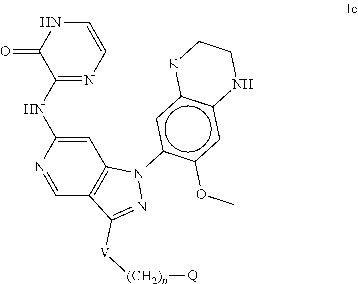

wherein
V is a divalent group selected from C(O)N(R₆);
Q is (C₃-C₆)heterocycloalkyl substituted by one (C₁-C₆)alkoxycarbonyl(CH₂)ₘ;
n is an integer selected from 1, 2, 3 and 4;
R₆ is in each occurrence independently selected from the group consisting of H, (C₁-C₆)alkyl;
single enantiomers, diastereoisomers and mixtures thereof
or a pharmaceutically acceptable salt or solvate thereof.

According to specific embodiments, the present invention provides the compounds of examples 1-59, as listed in the table below, or pharmaceutical acceptable salts and solvates thereof.

| Example No | Chemical Name |
|---|---|
| 1 | ethyl 1-(2-(1-(6-methoxy-3,4-dihydro-2H-benzo[b][1,4]thiazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 2 | ethyl (1s,3s)-3-(((1-(6-methoxy-3,4-dihydro-2H-benzo[b][1,4]thiazin-7-yl)-6- |
| | (pyrazolo[1,5-a]pyrimidin-3-yl)-1H-pyrazolo[4,3-c]pyridin-3- |
| | yl)carbamoyl)oxy)cyclobutane-1-carboxylate |
| 3 | ethyl 2-(((1-(6-methoxy-3,4-dihydro-2H-benzo[b][1,4]thiazin-7-yl)-6- |
| | (pyrazolo[1,5-a]pyrimidin-3-yl)-1H-pyrazolo[4,3-c]pyridin-3- |
| | yl)carbamoyl)oxy)acetate |
| 4 | ethyl 1-(2-(((1-(6-methoxy-3,4-dihydro-2H-benzo[b][1,4]thiazin-7-yl)-6- |
| | (pyrazolo[1,5-a]pyrimidin-3-yl)-1H-pyrazolo[4,3-c]pyridin-3- |
| | yl)carbamoyl)oxy)ethyl)piperidine-4-carboxylate |
| 5 | 1-methoxy-2-methylpropan-2-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 6 | propyl N-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)-N-methylglycinate |
| 7 | 3-(dimethylamino)propyl 1-(6-methoxy-3,4-dihydro-2H-benzo[b][1,4]thiazin- |
| | 7-yl)-6-(pyrazolo[1,5-a]pyrimidin-3-yl)-1H-pyrazolo[4,3-c]pyridine-3- |
| | carboxylate |
| 8 | 3-(4-methylpiperazin-1-yl)propyl 1-(6-methoxy-3,4-dihydro-2H- |
| | benzo[b][1,4]thiazin-7-yl)-6-(pyrazolo[1,5-a]pyrimidin-3-yl)-1H-pyrazolo[4,3- |
| | c]pyridine-3-carboxylate |
| 9 | ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 10 | sec-butyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 11 | cyclopropyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 12 | cyclopentyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 13 | 2,2,2-trifluoroethyl 1-(2-(1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridine-3- |
| | carboxamido)ethyl)piperidine-4-carboxylate |
| 14 | 2-hydroxy-2-methylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 15 | 3-hydroxy-2,2-dimethylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 16 | 2-methoxyethyl 1-(2-(1-(6-methoxy-3,4-dihydro-2H-benzo[b][1,4]oxazin-7-yl)- |
| | 6-(pyrazolo[1,5-a]pyrimidin-3-yl)-1H-pyrazolo[4,3-c]pyridine-3- |
| | carboxamido)ethyl)piperidine-4-carboxylate |
| 17 | tetrahydro-2H-pyran-4-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 18 | (tetrahydro-2H-pyran-4-yl)methyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 19 | (4-methylmorpholin-2-yl)methyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 20 | 2-(dimethylamino)-2-methylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 21 | 1-methylazetidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 22 | 1-methylpyrrolidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 23 | (R)-1-methylpyrrolidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 24 | (S)-1-methylpyrrolidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 25 | 2-cyano-2-methylpropyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 26 | 1-methylpiperidin-4-yl 1-(3-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)propyl)piperidine-4-carboxylate |
| 27 | ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-3-carboxylate |
| 28 | ethyl 4-fluoro-1-(2-(1-(6-methoxy-3,4-dihydro-2H-benzo[b][1,4]oxazin-7-yl)- |
| | 6-(pyrazolo[1,5-a]pyrimidin-3-yl)-1H-pyrazolo[4,3-c]pyridine-3- |
| | carboxamido)ethyl)piperidine-4-carboxylate |
| 29 | methyl (R)-1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)pyrrolidine-3-carboxylate |
| 30 | methyl (S)-1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)pyrrolidine-3-carboxylate |
| 31 | ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)azetidine-3-carboxylate |
| 32 | ethyl (3aR,5r,6aS)-2-(2-(1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridine-3- |
| | carboxamido)ethyl)octahydrocyclopenta[c]pyrrole-5-carboxylate |
| 33 | ethyl (1R,5S,6r)-3-(2-(1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridine-3- |
| | carboxamido)ethyl)-3-azabicyclo[3.1.0]hexane-6-carboxylate |
| 34 | ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)-4-methylpiperidine-4-carboxylate |
| 35 | ethyl 2-(1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidin-4-yl)acetate |
| 36 | ethyl 4-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)-1-methylpiperazine-2-carboxylate |
| 37 | methyl 2-(3-(((2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)(methyl)amino)methyl)phenyl)acetate |
| 38 | ethyl 1-(2-(1-(7-methoxy-1,2,3,4-tetrahydroquinolin-6-yl)-6-(pyrazolo[1,5- |
| | alpyrimidin-3-yl)-1H-pyrazolo[4,3-c]pyridine-3-carboxamido)ethyl)piperidine- |
| | 4-carboxylate |
| 39 | ethyl 2-(((1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3- |
| | yl)carbamoyl)oxy)acetate |
| 40 | ethyl (1s,3s)-3-(((1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3- |
| | yl)carbamoyl)oxy)cyclobutane-1-carboxylate |
| 41 | ethyl 1-(2-(((1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridin-3- |
| | yl)carbamoyl)oxy)ethyl)piperidine-4-carboxylate |
| 42 | 1-methylpiperidin-4-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 43 | ethyl (R)-1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)pyrrolidine-3-carboxylate |
| 44 | ethyl (S)-1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)pyrrolidine-3-carboxylate |
| 45 | isopropyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carboxylate |
| 46 | (4-methylmorpholin-2-yl)methyl 1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxylate |
| 47 | 3-cyanocyclobutyl 1-(2-(1-(6-methoxy-3,4-dihydro-2H-benzo[b][1,4]oxazin-7- |
| | yl)-6-(pyrazolo[1,5-a]pyrimidin-3-yl)-1H-pyrazolo[4,3-c]pyridine-3- |
| | carboxamido)ethyl)piperidine-4-carboxylate |
| 48 | 2-hydroxy-2-methylpropyl 2-(1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidin-4-yl)acetate |
| 49 | 2-cyano-2-methylpropyl (R)-1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)pyrrolidine-3-carboxylate |
| 50 | (3S,4S)-4-fluoro-1-methylpyrrolidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 51 | (3R*,4R*)-4-fluoro-1-methylpyrrolidin-3-yl 1-(2-(1-(6-methoxy-3,4-dihydro- |
| | 2H-benzo[b][1,4]oxazin-7-yl)-6-(pyrazolo[1,5-a]pyrimidin-3-yl)-1H- |
| | pyrazolo[4,3-c]pyridine-3-carboxamido)ethyl)piperidine-4-carboxylate |
| 52 | (R)-1-(2-fluoroethyl)pyrrolidin-3-yl 2-(1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidin-4-yl)acetate |
| 53 | (1-cyanocyclopropyl)methyl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 54 | 3-cyanopropyl 1-(2-(1-(6-methoxy-3,4-dihydro-2H-benzo[b][1,4]oxazin-7-yl)- |
| | 6-(pyrazolo[1,5-a]pyrimidin-3-yl)-1H-pyrazolo[4,3-c]pyridine-3- |
| | carboxamido)ethyl)piperidine-4-carboxylate |
| 55 | (R)-1-(2-fluoroethyl)pyrrolidin-3-yl (R)-1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)pyrrolidine-3-carboxylate |
| 56 | (R)-1-methylpyrrolidin-3-yl (S)-1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)pyrrolidine-3-carboxylate |
| 57 | 1-isopropylazetidin-3-yl 1-(2-(1-(6-methoxy-3,4-dihydro-2H- |
| | benzo[b][1,4]oxazin-7-yl)-6-(pyrazolo[1,5-a]pyrimidin-3-yl)-1H-pyrazolo[4,3- |
| | c]pyridine-3-carboxamido)ethyl)piperidine-4-carboxylate |
| 58 | ethyl 1-(2-(1-(6-methoxy-3,4-dihydro-2H-benzo[b][1,4]oxazin-7-yl)-6-((3-oxo- |
| | 3,4-dihydropyrazin-2-yl)amino)-1H-pyrazolo[4,3-c]pyridine-3- |
| | carboxamido)ethyl)piperidine-4-carboxylate |
| 59 | S-ethyl 1-(2-(1-(6-methoxy-3,4-dihydro-2H-benzo[b][1,4]oxazin-7-yl)-6- |
| | (pyrazolo[1,5-a]pyrimidin-3-yl)-1H-pyrazolo[4,3-c]pyridine-3- |
| | carboxamido)ethyl)piperidine-4-carbothioate |

The compounds of the invention showed high biochemical potency on JAKs targets (JAK1, JAK2, JAK3 and Tyk2) and high potency on a representative functional assay in cells (such as inhibition of pSTAT6 in BEAS cells stimulated with IL-13).

Preferred compounds showed a drop of at least 10 fold or more in the functional activity (such as inhibition of pSTAT6 in BEAS cells stimulated with IL-13) of the predicted carboxylic acid metabolite. More preferred compounds showed a drop even higher than 100 fold, even more preferred higher than 200 fold.

Even more preferred are compounds according to the invention that, in addition to the previous properties, show an high clearance at least in in-vitro stability assays in human and/or mice representative tissues as for example liver microsomes, and/or hepatocytes, and/or plasma, with an half-life less than or equal to 30 min using well established and validated assays.

Those assays were adapted from literature methods (Kevin J. Coe & Tatiana Koudriakova, Metabolic Stability Assessed by Liver Microsomes and Hepatocytes, Methods in Pharmacology and Toxicology, 2013; L Di, EH Kerns, Y Hong, H Chen, Development and application of high throughput plasma stability assay for drug discovery, International journal of pharmaceutics, 2005) and validated respect to generally accepted literature references (i.e. verapamil for human/mice microsomes/hepatocytes and propantheline for plasma stability in human/mice).

During lead optimization, high turnover in tissue stability tests is commonly deemed predictive of an high systemic clearance in-vivo that may further contribute to limit the systemic exposure after inhalation, making these preferred compounds particularly suited for inhaled administration.

The compounds of the invention, including all the compounds hereabove listed, can be prepared from readily available starting materials using general methods and procedures as described in the experimental part below or by using slightly modified processes readily available to those of ordinary skill in the art. Although a particular embodiment of the present invention may be shown or described herein, those skilled in the art will recognize that all embodiments or aspects of the present invention can be prepared using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures. Processes of preparation described below and reported in the following schemes should not be viewed as limiting the scope of the synthetic methods available for the preparation of the compounds of the invention.

In some cases, a step is needed in order to mask or protect sensitive or reactive moieties, generally known protective groups (PG) could be employed, in accordance with general principles of chemistry (Protective group in organic syntheses, 3rd ed. T. W. Greene, P. G. M. Wuts).

Compounds of formula I-1 and I-2, here reported again for clarity, including all here above listed, can be usually prepared according to the procedures shown in the schemes below. Where a specific detail or step differs from the general schemes it has been detailed in the specific examples, and/or in additional schemes.

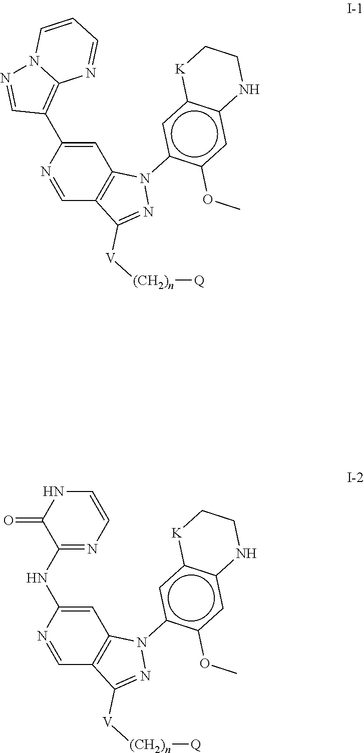

Compounds of formula (I-1) can be prepared according to scheme 1 starting from intermediates of formula IIa (or IIb, or IIc) by removing of PG₁ by mean of procedures well known to those skilled in the art. A suitable protective group for protecting secondary amines of intermediates Ila (or IIb, or IIc) can be carbamate type protective groups such as Boc (tert-butoxycarbonyl). Boc group can be easily removed by treating Boc protected intermediate IIa (or IIb, or IIc) in acidic conditions with an organic or an inorganic strong acid. For example, Boc group can be cleaved by treating the intermediates with trifluoroacetic acid neat or in mixture with an organic solvent such as DCM, DCE, THF or similar, typically at room temperature overnight.

The group Q′ present in intermediates of formula IIa (or IIb, or IIc) is a group of formula Q that can undergo a single step functional group interconversion to generate a compound of formula I-1. Example 43, 44 and 45 were prepared from example 29, 30 and 9 respectively by transesterification with an appropriate alcohol to provide the desired ester. A transesterification reaction can be carried out by reacting parent ester with an excess of the desired alcohol, in the presence of a strong inorganic acid such as sulfuric acid or hydrochloric acid, by heating at high temperature or up to boiling point of the alcohol.

It is apparent that in the case such conversions are not needed (when Q′ is already Q and/or PG₁ is H), any general approach described below for the preparation of intermediate IIa (or IIb, or IIc) will provide a compound of general formula I-1.

Intermediate IIa (or IIb, or IIc) can be obtained by direct introduction of pyrazolo[1,5-a]pyrimidin-3-yl moiety through a metal/palladium catalyzed cross coupling reaction such as Suzuki coupling, Stille coupling or similar (Strategic application of named reactions in organic synthesis, L. Kurti, B. Czako, Ed. 2005) by reaction of intermediate IIIa (or respectively IIIb or IIIc) with intermediate VI. For example, a suitable palladium catalyzed cross coupling for introducing pyrazolo[1,5-a]pyrimidin-3-yl moiety can be performed by reacting intermediate IIIa (or IIIb, or IIIc) with the corresponding boronic acid or boron pinacolate (intermediate VI, where A is dihydroxyboryl or 4,4,5,5-tetramethyl-1,3,2-dioxaborolanyl) in the presence of a palladacycle precatalyst as XPhos-Pd-G3 [(2-Dicyclohexylphosphino-2′,4′,6′-triisopropyl-1,1′-biphenyl)[2-(2′-amino-1,1′-biphenyl)]palladium(II) methanesulfonate], or in the presence of Pd catalyst such as tetrakistriphenylphosphinepalladium(0) or PdCl₂(dppf)₂, in an organic solvent such as THF, 1,4-dioxane, 1,2-dimethoxyethane, 2-propanol or DMF, with or without water, in the presence of an inorganic base like K₃PO₄ or Cs₂CO₃, under heating (typically in the range of 50-100° C.) for few hours (typically 1 to 5 h). Boronic acid and boronic pinacolate esters are generally commercially available or may be readily prepared by those skilled in the art starting from commercially available reagents.

Intermediates of formula IIIa may be obtained from intermediate IVa by means of amide coupling with the corresponding amine Va. An amide coupling can be performed by reacting the amine and the acid in an organic solvent like DMF, DCM, or THF, in the presence of a coupling agent like HATU((1-[Bis(dimethylamino)methylene]-1H-1,2,3-triazolo[4,5-b]pyridinium 3-oxide hexafluorophosphate), HBTU (O-(Benzotriazol-1-yl)-N,N,N′,N′-tetramethyluronium hexafluorophosphate) or COMU ((1-Cyano-2-ethoxy-2-oxoethylidenaminooxy)dimethylamino-morpholino-carbenium) and in the presence of an organic base like DIPEA, TEA, or pyridine.

Intermediate of formula IIIb may be obtained from intermediate IVa and an alcohol intermediate Vb through a two steps/one pot process that involves: 1) Acylazide formation and Curtius rearrangement to give an isocyanate intermediate and 2) one pot reaction of isocyanate with alcohol Vb to form the corresponding carbamate. The corresponding acyl azide of intermediate IIIb can be prepared by reaction with an azide source as azido(trimethyl)silane, in the presence of a suitable coupling agents such as T3P and with an organic base like TEA or DIPEA, in an organic solvent such as 2-methyl-THF, DMF or toluene. Subsequent Curtius rearrangement is done by thermal degradation around solvent reflux temperature (generally from 50° C. to 100° C.) for few hours (generally 1-3 hours) to give the corresponding isocyanate; after isocyanate formation, addition of alcohol intermediate Vb and continuing reflux overnight lead to the formation of carbamate of formula IIIb. In some cases, reaction of IVa with intermediate Vb can lead also to formation of intermediate IIIc that can be isolated from the same reaction together with intermediate IIIb.

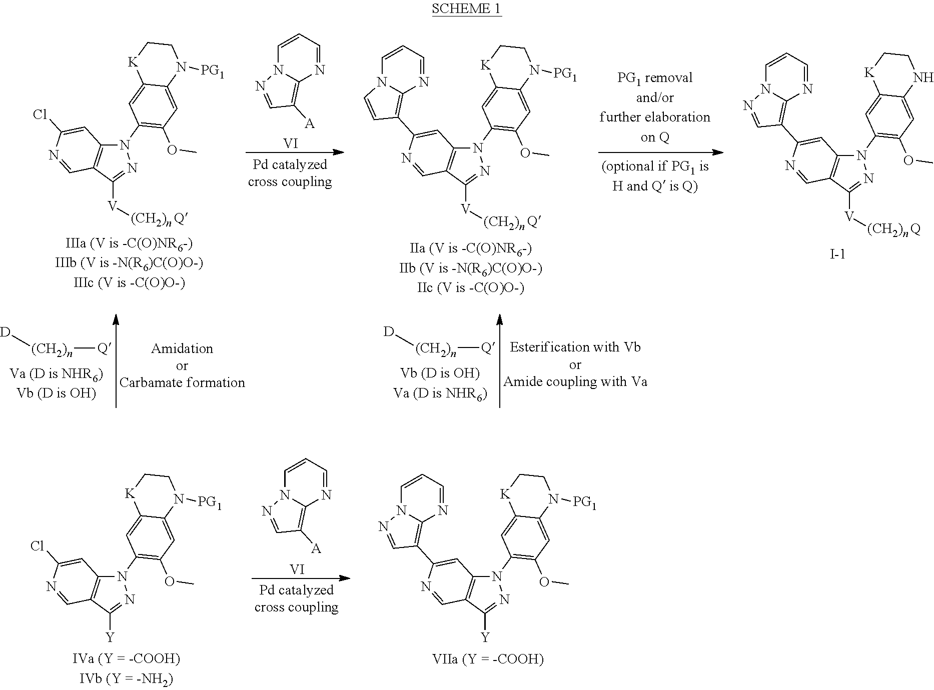

In another approach, intermediate IIIb may be prepared from intermediate IVb and intermediate Vb by a two steps/one pot process that involve: 1) isocyanate formation and 2) one pot reaction with alcohol Vb to form the corresponding carbamate. Intermediate IVb can be reacted with bis(trichloromethyl)carbonate in an organic solvent as DCM or THF, at low temperature like dry ice temperature (−78° C.), in the presence of an organic base like TEA or DIPEA for times up to 1 or 2 hours to form the corresponding isocyanate; addition of intermediate Vb and reaction at room temperature lead to the formation of carbamate of formula IIIb.

In a different approach, intermediate IIa may be obtained from intermediate VIIa and intermediate Va by an amide coupling using the same procedure described above for intermediate IIIa from IVa and Va.

In a different approach, intermediate IIc may be obtained from intermediate VIIa and intermediate Vb by esterification reaction promoted by a coupling agent like EDC (1-etil-3-(3-dimetilamminopropil)carbodiimmide) or DIC (N,N′-diisopropilcarbodiimmide), in an organic solvent as DCM or THF, in the presence of an organic base like DMAP or pyridine, at room temperature for few hours (typically 2-4 hours). Intermediate VIIa can be obtained from intermediate IVa and VI by a Suzuki coupling in a similar way to that described above for the reaction of intermediate IIIa (or respectively IIIb or IIIc) with an intermediate VI.

Intermediate IVa (or IVb) can be prepared according to scheme 2. Intermediate IVa (or IVb) can be prepared by means of N-arylation of intermediate VIIIa (or VIIIb) with a halide intermediate IX by using copper catalyzed Ullmann type reaction. An Ullmann reaction between a NH heteroaryl and an aryl/heteroaryl halide (bromide or iodide) can be performed in the presence of a suitable copper (I) catalyst/promoter such as CuI, Cu₂O or CuTC (copper thiophene carboxylate), ligandless or with a suitable ligand such as N,N-dimethylglicine, proline, phenantroline or dimethylcyclohexane-1,2-diamine (DMCHA), in the presence of an inorganic base such as K₂CO₃ or Cs₂CO₃, by heating (typically 90-150° C.) in a polar organic solvent such as DMSO, DMF or DMA, overnight or longer.

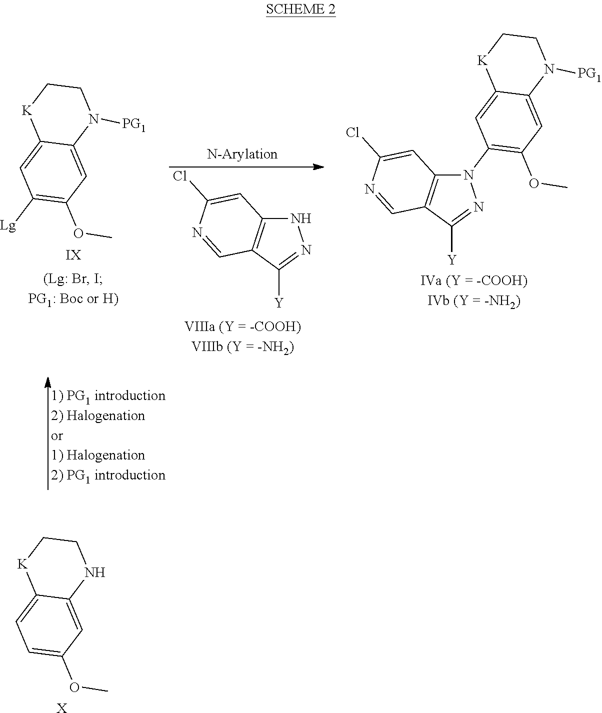

Intermediate IX may be obtained from intermediate X in a two steps process that involves PG₁ introduction (Boc group introduction) and halogenation or inverting the sequence of the steps. Insertion of Boc group can be carried out by reacting the anilino intermediate with Boc₂O in an organic solvent like THF or DCM, in the presence of an organic base like DMAP or pyridine, at room temperature for several hours up to overnight (typically 12 hours). Halogenation reaction can be carried out by reacting the aryl derivative with a source of electrophilic bromine or iodine such as 1,3-Dibromo-5,5-dimethyl-imidazolidine-2,4-dione, N-bromosuccinimide or N-iodosuccinimide, in an organic solvent like ethyl acetate, DMF, or DCM, at temperature around 0° C. or higher (up to 40° C.).

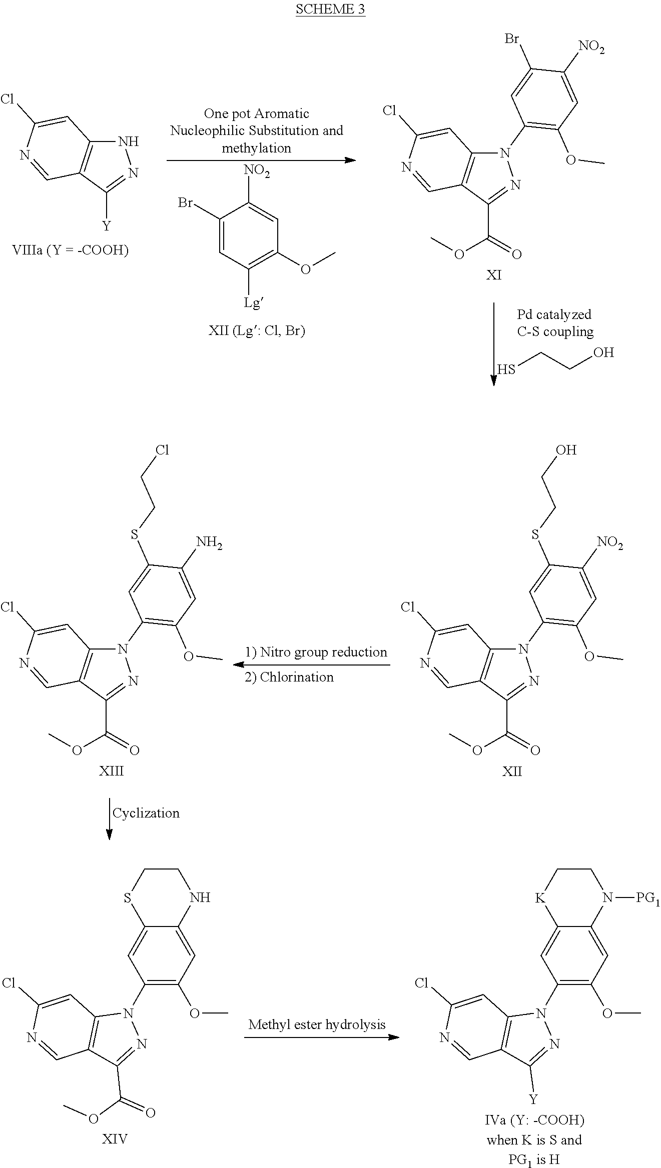

In another approach, intermediate IVa, where K is S and PG₁ is H, may be obtained from intermediate VIIIa by means of a multistep synthetic sequence reported in scheme 3. Intermediate VIIIa and intermediate XII can undergo an aromatic nucleophilic substitution (SNAr) to give intermediate XI, for example by reacting them in an organic solvent like DMF or 1,4-dioxane, in the presence of an organic base like DBU or DIPEA, at RT for few hours (typically 4 hours); addition of methyl halide like Mel at the end of SNAr reaction can lead to in situ methylation of the carboxylic acid and formation of intermediate XI. Intermediate XI can be reacted with mercaptoethanol under Pd-catalyzed C—S coupling conditions to give intermediate XII. C—S coupling can be performed by reacting aryl bromide XI and mercaptoethanol in the presence of a suitable catalytic system like as Pd₂(dba)₃/Xantphos or another suitable palladium source/phosphine source, in an organic solvent as 1,4-dioxane, toluene or DMA, in the presence of an organic base like DIPEA or DBU, at temperature up to 100° C. for few hours (typically 3-5 hours). Intermediate XII can be converted into intermediate XIII by a two steps process that involves: 1) nitro reduction and 2) chlorination. Nitro reduction can be performed by treating intermediate XII with a reducing agent like iron powder or zinc powder, in an organic solvent like methanol or ethanol, in the presence of an aqueous solution of a weakly acid inorganic salt like ammonium chloride, by heating at temperature up to 80° C. for few hours (typically 4-5 hours). Subsequent conversion of alcohol to the corresponding chloride can be achieved by reaction with neat thionyl chloride or oxalyl chloride at low temperature (around 0° C.). Chloride intermediate XIII can be cyclized to give intermediate XIV by heating (typically 80-100° C.) in an appropriate organic solvent like DMF or 1,4-dioxane, in presence of an inorganic base like potassium carbonate or sodium carbonate and in the presence of an additive like sodium iodide. Finally, intermediate XIV can be converted to intermediate IVa (where K is S and PG₁ is H) by ester hydrolysis performed by treating the methyl ester in a water-miscible organic solvent like THE or methanol, in the presence of an aqueous solution of an inorganic base like lithium hydroxide or sodium hydroxide, for few hours (typically 2-4 hours) at temperature up to 40° C.

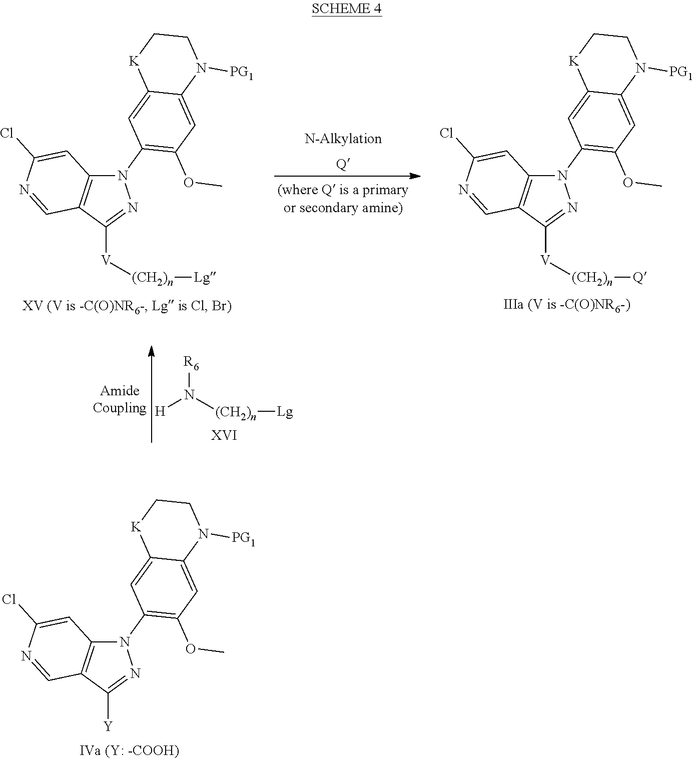

In another approach (scheme 4), intermediate IIIa can be prepared from intermediate XV by displacement of Lg″ by alkylation with primary/secondary nitrogen present in Q′ moiety by treating at room temperature for few hours (typically 5-7 hours) the amine Q′ and halide XV in an organic solvent like DMF or 1,4-dioxane, in the presence of an organic base like DIPEA or TEA. Intermediate XV can be obtained from intermediate IVa and XVI by an amide coupling reaction using similar conditions to those reported in scheme 1 for the conversion of intermediate IVa and intermediate Va to intermediate IIIa.

In a different approach (scheme 5), intermediate IIIa′ (or IIIb′) can be prepared from esterification with an alcohol of formula r-OH with the corresponding acid of formula XVIIa (or XVIIb) by using similar conditions to those reported in scheme 1 for the transformation of intermediates VIIa and Vb into IIc.

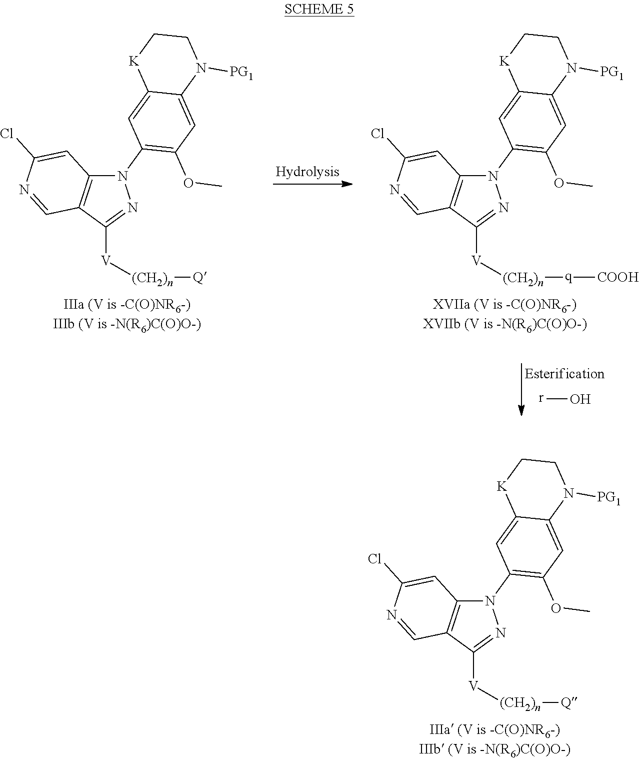

Intermediate XVIIa (or XVIIb) can be obtained from corresponding intermediate IIIa (or IIIb), where Q′ is a methyl ester (Q′ is q-COOMe) or ethyl ester (Q′ is q-COOEt), by hydrolysis in basic conditions in analogy to what described in scheme 4 for the conversion of intermediate XIV to intermediate IVa. The group Q″ present in intermediates of formula IIIa′ (or IIIb′) is a group that can undergo a single step functional group interconversion and/or PG deprotection to give a group of formula Q. Intermediates IIIa′ (or IIIb′) can be converted to compound of formula I-1 by using similar methods reported in scheme 1 to convert intermediate IIIa (or IIIb) to compound of formula I-1.

In a different approach (scheme 6), when group Q′ in intermediates of formula Ila contain a methyl ester or an ethyl ester, Ila can be transformed in IIa′ by a two steps process can allow transformation of group Q′ to Q″. In the first step, ester hydrolysis of Q′ of IIa generates an acid intermediate that can be submitted to an esterification with a proper alcohol (r-OH) to give intermediate IIa′. Methyl ester or ethyl ester hydrolysis can be carried out by reacting ester derivative with LiOH, NaOH or a suitable inorganic hydroxide in a mixture of water with THE, DMF or a suitable polar organic solvent. The acid can be transformed in an ester by activation of acid with a proper coupling agent like 2,4,6-trichlorobenzoyl chloride or HATU, followed by reaction with the corresponding alcohol in the presence of an organic base like TEA, DMAP or DIEA in an organic polar solvent like DMF. This procedure can also apply to the preparation of thioesters by replacing the alcohol (r-OH) with the proper thiol derivative (r-SH).

In another approach, compound of formula I-1, when Q contain a terminal CN group, may be prepared from the corresponding intermediate IIa that contain in Q′ a leaving group such as Br or Cl and alkylated with an inorganic cyanide salt like NaCN or KCN in an organic polar solvent like DMF by heating at temperature around 80° C. or higher.

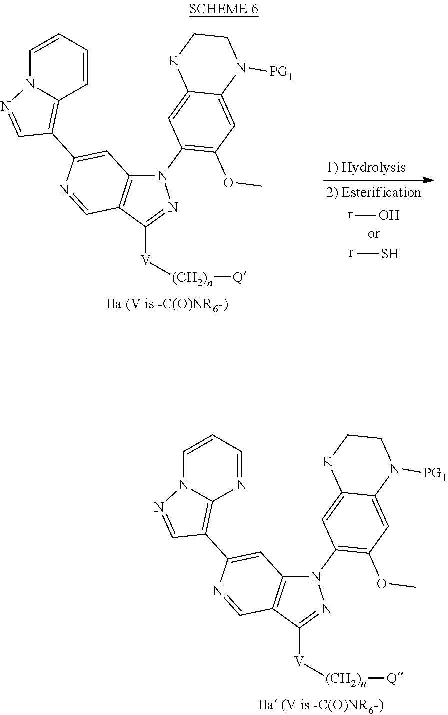

Compound of formula I-2 can be prepared like to compounds of formula I-1 by using similar synthetic sequences by introducing pyrazin-2 (1H)-one-3-aminyl moiety instead of pyrazol[1,5-a]pyrimidine-3-yl moiety. An example of such scheme modifications is provided by scheme 7. A suitable palladium catalyzed cross coupling for introducing pyrazin-2 (1H)-one-3-aminyl moiety is a Buchwald-Hartwig coupling. For synthetic convenience the lactam group of (3-oxo-3,4-dihydropyrazin-2-yl) amino needs to be masked as an alkoxyimmino group, where PG₂ is methyl, (such as a methoxy immino, —C(OMe)=N—) subsequently deprotected at the end of the synthesis of intermediate XIX. Intermediate IVa and intermediate XVIII can be reacted, to give intermediate XIX, in the presence of a suitable ligand palladacyle system such as XPhos-Pd-G3 (2-Dicyclohexylphosphino-2′,4′,6′-triisopropyl-1, 1′-biphenyl)[2-(2′-amino-1,1′-biphenyl)]palladium (II) methanesulfonate) or RuPhos-Pd-G3 (2-Dicyclohexylphosphino-2′,6′-diisopropoxy-1,1′-biphenyl) [2-(2′-amino-1,1′-biphenyl)]palladium(II) methanesulfonate) or in general a suitable Pd source (for example Pd₂(dba)₃ or Pd(OAc)₂) with a suitable phosphine ligand such as biphenylphosphine ligand type (RuPhos, X-Phos, or similar), in the presence of a strong organic base such as sodium tert-butoxide or an inorganic base such as Cs₂CO₃, in an organic solvent such 1,4-dioxane, THF or toluene, under heating at high temperature (typically 80-120° C.), for a few hours (typically overnight). Deprotection of PG₂ from intermediate XIX to give intermediate XX can be carried out by treatment of the protected precursor with TMS-C₁ (trimethylsilyl chloride) and sodium iodide in acetonitrile for 1 to 5 h at 60-100° C.; under these conditions also PG₁ when it is Boc can be deprotected. Finally, compound of formula I-2 can be prepared by reaction of intermediate XX with intermediate Va under amide coupling conditions similarly to what described in scheme 1 for the preparation of IIIa from IVa and Va.

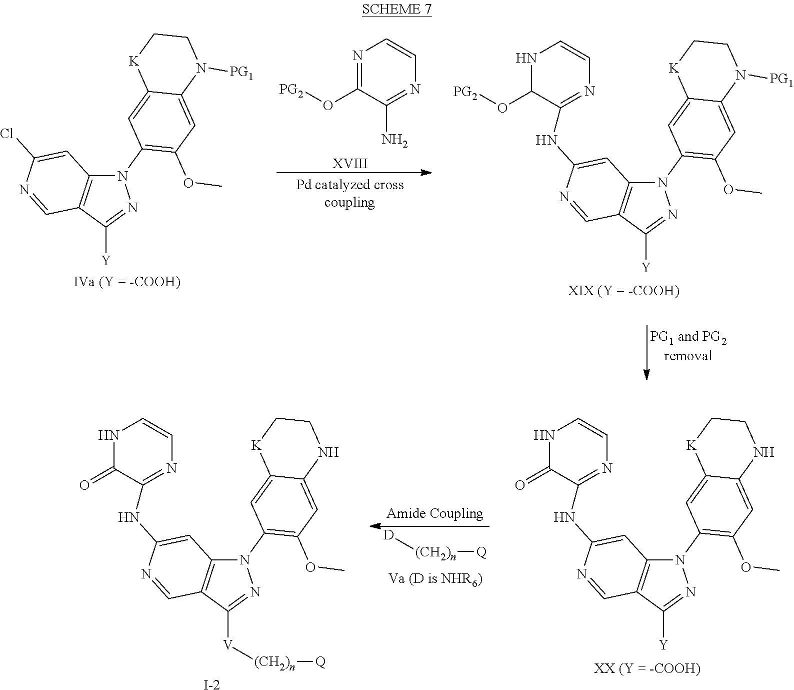

As herein described in detail, the compounds of the invention are inhibitors of kinase activity, in particular inhibiting JAK kinase activity for the treatment of JAK-dependent diseases.

In one aspect the invention provides compounds according to the invention, i.e. a compound of formula (I) or a pharmaceutical composition thereof, for use as a medicament, preferably for the prevention and/or treatment of respiratory and specifically pulmonary disease.

In a further aspect the invention provides the use of a compound (I), or a pharmaceutically acceptable salt thereof, in the manufacture of a medicament for the treatment of disorders associated with JAK mechanisms, particularly for the treatment of disorders such as respiratory and pulmonary diseases.

In particular the invention provides compounds of formula (I) for use in the prevention and/or treatment of pulmonary disease selected from the group consisting of asthma, chronic obstructive pulmonary disease COPD, idiopathic pulmonary fibrosis (IPF) acute lung injury and acute respiratory distress syndrome (ARDS).

Moreover, the invention provides a method for the prevention and/or treatment of disorders associated with JAK mechanisms, said method comprising administering to a patient in need of such treatment a therapeutically effective amount of a compound of the invention.

In particular, the invention provides methods for the prevention and/or treatment wherein the disorder is a respiratory disease selected from asthma, chronic obstructive pulmonary disease (COPD), idiopathic pulmonary fibrosis (IPF), acute lung injury and acute respiratory distress syndrome (ARDS).

Preferred is the use of the compounds of the invention for the prevention of the aforesaid disorders.

Equally preferred is the use of the compounds of the invention for the treatment of the aforesaid disorders.

Generally speaking, compounds which are JAK inhibitors may be useful in the treatment of many disorders associated with JAK enzyme mechanisms.

In one embodiment, the disorder that can be treated by the compound of the present invention is selected from the group consisting of asthma, chronic obstructive pulmonary disease (COPD) and interstitial lung disease such as idiopathic pulmonary fibrosis (IPF), acute lung injury and acute respiratory distress syndrome (ARDS).

In a further embodiment, the disorder is selected from asthma and chronic obstructive pulmonary disease (COPD).

The methods of treatment of the invention comprise administering an effective amount of a compound of formula (I) or a pharmaceutically acceptable salt thereof to a patient in need thereof. As used herein, “effective amount” in reference to a compound of formula (I) or a pharmaceutically acceptable salt thereof or other pharmaceutically active agent means an amount of the compound sufficient to treat the patient's condition but low enough to avoid serious side effects and it can nevertheless be routinely determined by the skilled artisan. The compounds of formula (I) or pharmaceutically acceptable salts thereof may be administered once or according to a dosing regimen wherein a number of doses are administered at varying intervals of time for a given period of time. Typical daily dosages may vary depending upon the particular route of administration chosen.

The invention also provides pharmaceutical compositions of compounds of formula (I) in admixture with one or more pharmaceutically acceptable carrier or excipient, for example those described in Remington's Pharmaceutical Sciences Handbook, XVII Ed., Mack Pub., N.Y., U.S.A.

The present invention is also directed to use of the compounds of the invention and their pharmaceutical compositions for various route of administration.

Administration of the compounds of the invention and their pharmaceutical compositions may be accomplished according to patient needs, for example, orally, nasally, parenterally (subcutaneously, intravenously, intramuscularly, intrasternally and by infusion), by inhalation, rectally, vaginally, topically, locally, transdermally, and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like. Time release capsules, tablets and gels are also advantageous.

Various liquid oral dosage forms can also be used for administering compounds of the invention, including aqueous and non-aqueous solutions, emulsions, suspensions, syrups, and elixirs. Such dosage forms can also contain suitable known inert diluents such as water and suitable known excipients such as preservatives, wetting agents, sweeteners, flavorants, as well as agents for emulsifying and/or suspending the compounds of the invention. The compounds of the present invention may be formulated as injectable composition, for example to be injected intravenously, in the form of an isotonic sterile solution. Other preparations are also possible.

Suppositories for rectal administration of the compounds of the invention can be prepared by mixing the compound with a suitable excipient such as cocoa butter, salicylates and polyethylene glycols.

Formulations for vaginal administration can be in the form of cream, gel, paste, foam, or spray formula containing, in addition to the active ingredient, such as suitable carriers, are also known.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Compounds of the invention may exhibit profile suitable for oral route administration. Optimization of drugs for oral delivery needs certain characteristics that allow orally administered compound to be absorbed by GI (gastrointestinal) tract and to be poorly cleared in order to give a good bioavailability (F %), thus to maintain a sufficient concentration in plasma and target tissues for a time adequate to sustain pharmacological effect. To enhance oral bioavailability, one or more features of the compounds need to be optimized such as, and not limited to, maximizing membrane permeability and reducing metabolic hot spots (optimizing in-vitro clearance).

For the treatment of the diseases of the respiratory tract, the compounds according to the invention, as above said, may also preferably be administered by inhalation.

Some preferred compounds of the invention exhibit profile suitable for inhalatory route administration.

Drugs optimized for inhaled delivery require certain characteristics that allow the compound, when administered to the lung to maintain a sufficient local concentration (lung retention) to exert a pharmacological effect of the desired duration, with minimal drug absorption in the GI tract for the swallowed fraction and in general non-relevant levels in unwanted compartments (i.e. plasma). For this purpose, one or more features of a compounds were optimized such as, and not limited to, membrane permeability, dissolution rate and the compound's basicity to enhance its binding to the phospholipid-rich lung tissue or through lysosomal trapping. In some embodiments, compounds of invention show one or more of the features above in the range desirable for an inhaled compound. inhalation, a JAKi should preferably possess additional properties that may further limit the systemic exposure after inhalation.

A way to limit systemic exposure, after local administration, might be to design soft-drug that means the introduction of specific moieties, like for example esters in the present invention, which favour a controlled rapid systemic metabolism (in the liver and/or in the blood) into predicted functionally less active, or even negligibly active, metabolites compared to parent compound. For this purpose, one way resulted in the optimization of ‘suitably designed esters derivatives’ which having corresponding predicted carboxylic acid metabolites showing a drop in functional activity (like cell based activity). Further advantageously, such optimized esters can be substrates of liver and/or blood esterases that may be beneficial for achieving an enhanced clearance in-vivo.

Thus, preferred compounds of the invention showed one or more of the following properties: high biochemical activity on target, strong functional activity (like cell based activity), a rapid clearance in representative assays (stability in liver microsomes and/or hepatocytes, plasma stability) and drop in the functional activity of the predicted carboxylic acid metabolite so that they can have potential for giving compounds with an improved safety.

Inhalable preparations include inhalable powders, propellant-containing metering aerosols or propellant-free inhalable formulations and may be administered through a suitable inhalation device which may be respectively selected from dry powder inhaler, pressurized metered dosed inhaler, or a nebulizer.

For administration as a dry powder, single- or multi-dose inhalers known from the prior art may be utilized. In that case the powder may be filled in gelatine, plastic or other capsules, cartridges or blister packs or in a reservoir.

A diluent or carrier, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in the form of solutions or suspensions in an aqueous, alcoholic or hydroalcoholic medium and they may be delivered by jet or ultrasonic nebulizers known from the prior art or by soft-mist nebulizers such as Respimat®, a registered trademark of Boehringer Ingelheim Pharmaceuticals (Wachtel, H., Kattenbeck, S., Dunne, S. et al. Pulm Ther (2017) 3:19.

The compounds of the invention, regardless of the route of administration, can be administered as the sole active agent or in combination (i.e. as co-therapeutic agents administered in fixed dose combination or in combined therapy of separately formulated active ingredients) with other pharmaceutical active ingredients.

The compounds of the invention can be administered as the sole active agent or in combination with other pharmaceutical active ingredients including those currently used in the treatment of respiratory disorders, and known to the skilled person, such as beta2-agonists, antimuscarinic agents, corticosteroids, mitogen-activated kinases (P38 MAP kinases) inhibitors, PI3K inhibitors (phosphoinositide 3-kinases), nuclear factor kappa-B kinase subunit beta inhibitors (IKK2), Rho kinase inhibitors (ROCKi), human neutrophil elastase (HNE) inhibitors, phosphodiesterase 4 (PDE4) inhibitors, leukotriene modulators, non-steroidal anti-inflammatory agents (NSAIDs) and mucus regulators).

The invention is also directed to a kit comprising the pharmaceutical compositions of compounds of the invention alone or in combination with or in admixture with one or more pharmaceutically acceptable carriers and/or excipients and a device which may be a single- or multi-dose dry powder inhaler, a metered dose inhaler or a nebulizer.

The dosages of the compounds of the invention depend upon a variety of factors including the particular disease to be treated, the severity of the symptoms, the route of administration, the frequency of the dosage interval, the particular compound utilized, the efficacy, toxicology profile, and pharmacokinetic profile of the compound.

A pharmaceutical composition comprising a compound of the invention suitable to be administered by inhalation is in various respirable forms, such as inhalable powders (DPI), propellant-containing metering aerosols (PMDI) or propellant-free inhalable formulations (e.g. UDV).

The invention is also directed to a device comprising the pharmaceutical composition comprising a compound according to the invention, which may be a single- or multi-dose dry powder inhaler, a metered dose inhaler and a nebulizer particularly soft mist nebulizer.

The following examples illustrate the invention in more detail.

The features of the invention will become apparent in the course of the following descriptions of exemplary embodiments which are given for illustration of the invention and are not intended to be limiting thereof.

### Preparation of Intermediates and Examples

### General Experimental Details

Chemical Names of the compounds were generated with Structure To Name Enterprise 10.0 Cambridge Software or latest.

Purification by ‘chromatography’ or ‘flash chromatography’ refers to purification using a Biotage SP1, or Interchim puriFlash purification system, or Biotage Isolera Four purification system equipped with Biotage Dalton 2000 mass detector, or equivalent MPLC using a pre-packed polypropylene column containing stationary phase (cartridge). Where products were purified using a Si cartridge, this refers to an Interchim (or equivalent) pre-packed polypropylene column containing unbounded activated silica with spherical particles with average size of 15 μm or Isolute® (or equivalent) pre-packed polypropylene column containing unbounded activated silica with irregular particles with average size of 50 μm. Fractions containing the desired product (identified by TLC and/or LCMS analysis) were pooled and concentrated in vacuo. Purification by ‘reverse phase chromatography’ or ‘reverse phase flash flash chromatography’ refers to purification on MPLC instrument with with C18 functionalized silica cartridges, as Biotage Sfar C₁₈ or equivalents. Where an SCX-2 cartridge was used, ‘SCX-2 cartridge’ refers to a Bond Elut® pre-packed polypropylene column containing a non-end-capped propylsulphonic acid functionalised silica strong cation exchange sorbent. Where preparative HPLC-MDAP was used for purification (MDAP: mass directed automatic purification) fractions containing the desired product were pooled and the solvent removed by evaporation or alternatively lyophilised.

### NMR Methods

NMR spectra were obtained on a Bruker Avance III 600 (5 mm RT inverse probe head), Bruker DRX 500, Bruker Avance AV 400 (5 mm RT direct probehead) or Bruker DPX 300 spectrometers using standard Bruker pulse sequences. Alternatively, NMR spectra were recorded with Varian MR-400 spectrometer operating at 400 Mhz or a Varian Unity Inova 400 spectrometer with a 5 mm inverse detection triple resonance probe operating at 400 MHz. DMSO-d₆ or CDCl₃ were used as solvents. Chemical shifts are given in relative to internal standard tetramethylsilane or solvent residual peak. All experiments were recorded at 298 K, unless stated differently. Coupling constants, (J values) are given in hertz (Hz) and multiplicities are reported using the following abbreviation: s=singlet, d=doublet, t=triplet, q=quartet, m=multiplet, br=broad, nd=not determined.

### LCMS Methods

### Method 1

Acquity UPLC coupled with SQD mass spectrometer; Column: Acquity UPLC BEH C₁₈ (50 mm×2.1 mm i.d., 1.7 μm packing diameter), mobile phase A: 0.1% formic acid in water, mobile phase B: 0.1% formic acid in acetonitrile;

| Gradient - Time | Flow (mL/min) | A % | B % |
|---|---|---|---|
| 0.00 | 0.9 | 97 | 3 |
| 1.50 | 0.9 | 3 | 97 |
| 1.90 | 0.9 | 3 | 97 |
| 2.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 2

Acquity UPLC coupled with SQD mass spectrometer; Column: Acquity UPLC BEH C₁₈ (50 mm×2.1 mm i.d., 1.7 μm packing diameter), mobile phase A: 10 mM aqueous solution of ammonium bicarbonate (adjusted to pH 10 with ammonia), mobile phase B: acetonitrile;

| Gradient-Time | Flow (mL/min) | A % | B % |
|---|---|---|---|
| 0.00 | 0.9 | 97 | 3 |
| 1.50 | 0.9 | 3 | 97 |
| 1.90 | 0.9 | 3 | 97 |
| 2.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 3

AGILENT LC 1260 Infinity with SFC and Agilent 6540 UHD Accurate-Mass Q-TOF LC/MS; Column: Acquity UPLC BEH C₁₈ (100 mm×2.1 mm i.d., 1.7 μm packing diameter), mobile phase A: 0.1% formic acid in water, mobile phase B: 0.1% formic acid in acetonitrile;

| Gradient-Time | Flow (mL/min) | A % | B % |
|---|---|---|---|
| 0.00 | 0.5 | 97 | 3 |
| 8.00 | 0.5 | 0 | 100 |
| 10.00 | 0.5 | 97 | 3 |
| 12.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1500 AMU.

### Method 4

AGILENT LC 1260 Infinity with SFC and Agilent 6540 UHD Accurate-Mass Q-TOF LC/MS; Column: Acquity UPLC BEH C₁₈ (100 mm×2.1 mm i.d., 1.7 μm packing diameter), mobile phase A: 0.05% aqueous ammonia, mobile phase B: acetonitrile;

| Gradient-Time | Flow (mL/min) | A % | B % |
|---|---|---|---|
| 0.00 | 0.5 | 97 | 3 |
| 8.00 | 0.5 | 0 | 100 |
| 10.00 | 0.5 | 97 | 3 |
| 12.00 | 0.05 | 97 | 3 |

Column temperature: 40° C.; UV detection: from 210 nm to 350 nm; MS conditions: Ionisation Mode: alternate-scan Positive and Negative Electrospray (ES⁺/ES−), Scan Range: 100 to 1000 AMU.

### Method 5

Shimadzu LCMS-2020 Single Quadrupole Liquid Chromatograph Mass Spectrometer; Column: Acquity UPLC BEH C₁₈ (100 mm×2.1 mm i.d., 1.7 μm packing diameter), mobile phase A: 0.1% formic acid in water, mobile phase B: 0.1% formic acid in acetonitrile;

| Gradient-Time | Flow (mL/min) | A % | B % |
|---|---|---|---|
| 0.10 | 0.5 | 80 | 20 |
| 2.00 | 0.5 | 5 | 95 |
| 2.70 | 0.5 | 5 | 95 |
| 2.80 | 0.5 | 80 | 20 |
| 3.00 | 0.5 | 80 | 20 |

Column temperature: 25° C.; UV detection: 215 nm and 254 nm; MS conditions: Ionisation Mode: alternate-scan Positive and Negative Electrospray (ES⁺/ES−), Scan Range: 100 to 1000 AMU.

### Method 6

Acquity UPLC coupled with QDA mass detector; Column: Acquity UPLC CSH C18 (50 mm×2.1 mm i.d., 1.7 μm packing diameter), mobile phase A: 0.05% (v/v) formic acid in water/MeCN 95/5, mobile phase B: 0.05% (v/v) formic acid in acetonitrile/water 95/5;

| Gradient - Time | Flow (mL/min) | A % | B % |
|---|---|---|---|
| 0.00 | 1.0 | 95 | 5 |
| 1.50 | 1.0 | 5 | 95 |
| 1.90 | 1.0 | 5 | 95 |
| 2.00 | 1.0 | 95 | 5 |
| 2.30 | 1.0 | 95 | 5 |

Column temperature: 40° C.; UV detection: from 210 nm to 350 nm; MS conditions: Ionisation Mode: alternate-scan Positive and Negative Electrospray (ES+/ES−), Scan Range: 100 to 1000 AMU.

### HPLC-MDAP

### Method 1

Agilent 1290 Infinity II Purification System; Column: Waters XBridge® (C18, 100 mm×19 mm i.d., 5 μm), mobile phase A: 0.1% (v/v) formic acid in water, mobile phase B: acetonitrile;

| Gradient - Time | Flow (mL/min) | A % | B % |
|---|---|---|---|
| 0.00 | 30 | 90 | 10 |
| 0.80 | 30 | 90 | 10 |
| 5.00 | 30 | 70 | 30 |
| 6.01 | 30 | 0 | 100 |
| 6.95 | 30 | 0 | 100 |
| 6.96 | 30 | 10 | 90 |

### Abbreviations Used:

Boc₂O=Di-tert-butyl dicarbonate; aq.=aqueous; DBU=1,8-Diazabicyclo[5.4.0]undec-7-ene; DCC=Dicyclohexylcarbodiimine; DCM=Dichloromethane; DIPEA=N,N-Diisopropylethylamine; DMAP=4-dimethylaminopyridine; DMCHDA=trans-N,N′-Dimethylcyclohexane-1,2-diamine; DMF=N,N-Dimethylformamide; DMSO=Dimethylsulfoxide; EDC·HCl=Ethyl-N,N-dimethylaminoethyl-carbodiimide, Hydrochloride; EtOAc=Ethyl acetate; HATU=(1-[Bis(dimethylamino)methylene]-1H-1,2,3-triazolo[4,5-b]pyridinium 3-oxid hexafluorophosphate); LCMS=Liquid chromatography-mass spectrometry; 1H-NMR=Proton nuclear magnetic resonance; RM=Reaction mixture; Rt=Retention time; RT=Room temperature; sat.=saturated; T3P®=Propylphosphonic anhydride; TEA=Triethylamine; TFA=Trifluoroacetic acid; THF=Tetrahydrofuran; Xphos-Pd-G3-(2-Dicyclohexylphosphino-2′,4′,6′-triisopropyl-1,1′-biphenyl) [2-(2′-amino-1,1′-biphenyl)]palladium(II) methanesulfonate

In the procedures that follow, some of the starting materials are identified through an “Intermediate” or “Example” number with indications on Step number. This is provided merely for assistance to the skilled chemist.

A “similar” or “analogous” procedure means that such a procedure may involve minor variations, for example reaction temperature, reagent/solvent amount, reaction time, work-up conditions and/or chromatographic purification conditions.

The stereochemistry of the compounds in the Examples, where indicated, has been assigned on the assumption that absolute configuration at resolved stereogenic centres of starting materials is maintained throughout any subsequent reaction conditions.

Unless otherwise stated, where absolute configuration (R) or(S) is reported in the compound name, ee % has to be considered equal or greater than 90%.

### PREPARATION OF INTERMEDIATES

### Intermediate 1

### Step 1

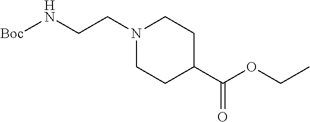 [SMILES: CCOC(=O)C1CCN(CCNC(=O)OC(C)(C)C)CC1]

### Ethyl 1-(2-((tert-butoxycarbonyl)amino)ethyl)piperidine-4-carboxylate (Intermediate 1-1)

A suspension of tert-butyl N-(2-bromoethyl)carbamate (3 g, 13.0 mmol), ethyl piperidine-4-carboxylate (2.1 mL, 13.0 mmol) and K₂CO₃ (3.7 g, 27 mmol) in DMF (30.0 mL) was stirred at 65° C. for 24 h. After cooling to RT, RM was diluted with water (80 mL) and extracted with EtOAc (3×80 mL). Combined organics were washed with sat. aq. NaHCO₃ (80 mL) and sat. aq. NaCl (50 mL). Organic solvent was removed in vacuo and residue was purified by flash chromatography on Si cartridge by eluting with 0-50% DCM: MeOH (93:7) in DCM to afford the title product (3.24 g).

LCMS (Method 2): Rt=1.05 min, ES⁺ m/z 301.3 [M+H]⁺.

### Step 2

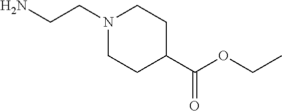

### Ethyl 1-(2-aminoethyl)piperidine-4-carboxylate (Intermediate 1)

A solution of intermediate 1-1 (1.77 g, 5.9 mmol) and TFA (8.8 mL, 118 mmol) in DCM (14 mL) was stirred at RT overnight. Volatiles were removed in vacuo and crude product passed through an SCX cartridge, washed with EtOH (500 mL) and eluted with 15% aq. NH₃ in EtOH (100 mL) to afford the title product (1.16 g).

¹H-NMR (300 MHz, CDCl₃) δ: 4.13 (q, J=6.9 Hz, 2H), 2.84-2.88 (m, 2H), 2.78 (t, J=6.2 Hz, 2H), 2.39 (t, J=6.2 Hz, 2H), 2.24-230 (m, 1H), 2.02 (t, J=11.5 Hz, 2H), 1.87-1.91 (m, 2H), 1.71-1.79 (m, 2H), 1.25 (t, J=6.9 Hz, 3H).

### Intermediate 2a

### Step 1

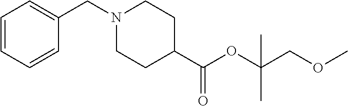 [SMILES: COCC(C)(C)OC(=O)C1CCN(Cc2ccccc2)CC1]

### 1-Methoxy-2-methylpropan-2-yl 1-benzylpiperidine-4-carboxylate (Intermediate 2a-1)

A solution of 1-benzylpiperidine-4-carboxylic acid (500 mg, 2.26 mmol) in DCM (15.0 mL) was treated with oxalyl dichloride (1.94 mL, 22.6 mmol) and DMF (52.2 μL, 0.68 mmol). RM was stirred at RT for 2 h, then volatiles were evaporated in vacuo. The residue was taken with DCM (5.0 mL), cooled to 10° C. and followed by the addition of 1-methoxy-2-methyl-propan-2-ol (1.32 mL, 11.3 mmol) and DIPEA (2.52 mL, 18.1 mmol). RM was stirred at RT for 16 h. RM was quenched with water (20 mL) and extracted with DCM (3×20 mL). Combined organic layers were passed through a phase separator and solvent removed in vacuo. The residue was purified by flash chromatography on Si cartridge by eluting with 0-50% DCM/MeOH (20:1) in DCM to give the desired product. (242 mg).

LCMS (Method 1): Rt=0.64 min, ES⁺ m/z 306.2 [M+H]⁺.

### Step 2

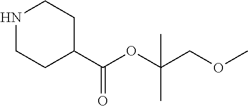

### 1-Methoxy-2-methylpropan-2-yl piperidine-4-carboxylate (Intermediate 2a)

A solution of intermediate 2a-1 (242 mg, 0.27 mmol) in MeOH (6 mL) was added with Pd/C (10%, 77.8 mg, 0.07 mmol) and kept under hydrogen atmosphere at 1 bar (baloon pressure) for 16 h. RM was filtered through a bed of diacetomatous earth and concentrated in vacuo. The residue was purified by flash chromatography on Si cartridge by eluting with 0-100% DCM/MeOH/NH₄OH (90:15:1.5) in DCM To give the title product (113 mg).

¹H-NMR (300 MHz, CDCl₃) δ: 3.48 (s, 2H), 3.34 (s, 3H), 3.05 (dt, J=12.5, 3.9 Hz, 2H), 2.60 (td, J=12.0, 2.6 Hz, 2H), 2.33 (tt, J=11.0, 3.8 Hz, 1H), 1.79-1.87 (m, 2H), 1.49-1.62 (m, 2H), 1.40 (s, 6H).

### Intermediate 2b

### Step 1

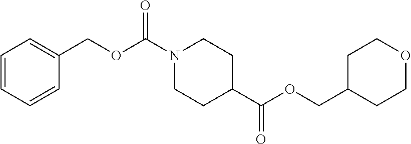 [SMILES: O=C(OCC1CCOCC1)C1CCN(C(=O)OCc2ccccc2)CC1]

### 1-benzyl 4-((tetrahydro-2H-pyran-4-yl)methyl) piperidine-1,4-dicarboxylate (Intermediate 2b-1)

1-phenylmethoxycarbonylpiperidine-4-carboxylic acid (300 mg, 1.14 mmol) was suspended in a mixture MeCN/DMF 5/2 (7.0 ml), then 4-(bromomethyl)tetrahydro-2H-pyran and cesium carbonate were added and the mixture was stirred at 40° C. for 18 h. The mixture was filtered, and the crude purified by chromatography on Si cartridge by eluting with hexane\EtOAc gradient to afford the desired product (300 mg).

LCMS (Method 6): Rt=1.09 min, ES⁺ m/z 362.2 [M+H]⁺

### Step 2

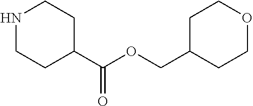 [SMILES: O=C(OCC1CCOCC1)C1CCNCC1]

### (Tetrahydro-2H-pyran-4-yl)methyl piperidine-4-carboxylate (Intermediate 2b)

A solution of intermediate 2b-1 (300 mg, 0.83 mmol) in EtOH (6 mL) was added with Pd/C (5%, 50 mg) and pH adjusted below 2 with aq. HBr 2M. RM was kept under hydrogen atmosphere for 3 h, then filtered through a pad of silica gel and concentrated to dryness to afford the title product (188 mg).

LCMS (Method 6): Rt=0.23 min, ES⁺ 228.28 m/z [M+H]⁺

### Intermediate 2c

### Step 1

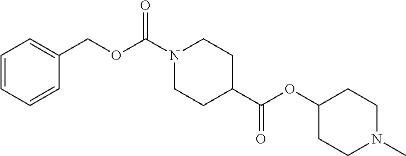 [SMILES: CN1CCC(OC(=O)C2CCN(C(=O)OCc3ccccc3)CC2)CC1]

### 1-benzyl 4-(1-methylpiperidin-4-yl)piperidine-1,4-dicarboxylate (Intermediate 2c-1)

1-((benzyloxy)carbonyl)piperidine-4-carboxylic acid (350 mg, 1.33 mmol, 82) was suspended in 5 mL of dry DCM, added with a drop of dry DMF and followed by dropwise addition oxalyl chloride (233 μl, 2.66 mmol). After 1h, RM was concentrated to dryness and taken with dry DCM (10 ml) and the solution added dropwise to a mixture of -methylpiperidin-4-ol (230 mg, 1.994 mmol) and DIPEA (697 μl, 3.99 mmol) in dry DCM. After 1h, RM was quenched by the addition of aq. sat. NaHCO₃ (20 ml) and the organic layer separated, dried over Na₂SO₄ and concentrated to dryness. The crude material was purified by chromatography on Si cartridge by eluting with 0-20% MeOH in DCM to afford the title product (391 mg).

LCMS (Method 6): Rt=0.55 min, ES⁺ 361.3 m/z [M+H]⁺

### Step 2

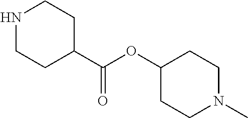 [SMILES: CN1CCC(OC(=O)C2CCNCC2)CC1]

### 1-methylpiperidin-4-yl piperidine-4-carboxylate (Intermediate 2c)

To a solution of intermediate 2c-1 (400 mg, 1.110 mmol) in EtOH (6 mL) was added Pd/C 5% (50 mg) and pH adjusted below 2 with aq. HCl 2M. The RM was kept under hydrogen atmosphere for 3 h, then filtered through a pad of silica gel and concentrated to dryness to afford the desired compound (230 mg).

LCMS (Method 6): Rt=0.17 min, ES+227.3 m/z [M+H]⁺

### Intermediate 3

### Step 1

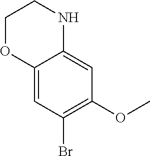

### 7-Bromo-6-methoxy-3,4-dihydro-2H-benzo[b][1,4]oxazine (Intermediate 3-1)

A solution of 6-methoxy-3,4-dihydro-2H-1,4-benzoxazine (3.0 g, 18.20 mmol) in EtOAc (30.0 mL) was cooled to 0° C. 1,3-Dibromo-5,5-dimethyl-imidazolidine-2,4-dione (2.6 g, 9.08 mmol) was added portion-wise over 15 minutes. RM was stirred for additional 30 min at 0° C. and quenched with an aqueous K₂CO₃ solution (10% w/w; 60 mL). The organic layer was separated, washed with sat. aq. NaCl and concentrated in vacuo. The residue was purified by flash chromatography on Si cartridge by eluting with 0-30% EtOAc in cyclohexane to afford the title product (3.5 g).

LCMS (Method 1): Rt=0.97 min, ES⁺ m/z 243.9/245.9 [M+H]⁺.

### Step 2

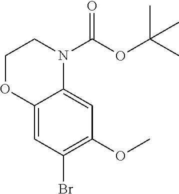 [SMILES: COc1cc2c(cc1Br)OCCN2C(=O)OC(C)(C)C]

### tert-Butyl 7-bromo-6-methoxy-2,3-dihydro-4H-benzo[b][1,4]oxazine-4-carboxylate (Intermediate 3)

THF (15 mL) was added to a mixture of intermediate 3-1 (1.4 g, 5.74 mmol), DMAP (840.9 mg, 6.88 mmol) and Boc₂O (2.80 g, 13.19 mmol), then RM stirred at RT overnight. RM was partitioned between EtOAc (50 mL)/water (30 mL). The organic layer was washed with 2M aq. citric acid (2×20 mL), sat. aq. NaCl (20 mL) and evaporated in vacuo. The residue was purified by flash chromatography on Si cartridge by eluting with 0-10% EtOAc in cyclohexane to afford the title compound (1.26 g).

LCMS (Method 1): Rt=1.27 min

¹H-NMR (300 MHz, CDCl₃) δ: 7.56 (brs, 1H), 7.05 (s, 1H), 4.14-4.18 (m, 2H), 3.82 (s, 3H), 3.78-3.82 (m, 2H), 1.53 (s, 9H).

### Intermediate 4

### Step 1

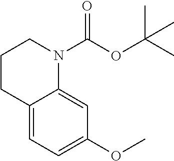 [SMILES: COc1ccc2c(c1)N(C(=O)OC(C)(C)C)CCC2]

### tert-Butyl 7-methoxy-3,4-dihydroquinoline-1 (2H)-carboxylate (Intermediate 4-1)

A solution of 7-methoxy-1,2,3,4-tetrahydroquinoline (500 mg, 3.06 mmol), DMAP (449 mg, 3.68 mmol) and Boc₂O (1.54 g, 7.05 mmol) in THF (10 mL) was stirred at RT overnight. A further equivalent of Boc₂O was added and stirring proceed at RT. RM was partitioned between EtOAc (50 mL) and water (30 mL). The organic layer was washed with aq. 2M citric acid (2×15 mL), sat. aq. NaCl (20 mL) and solvent evaporated in vacuo. The residue was purified by flash chromatography on Si cartridge by eluting with 0-10% EtOAc in cyclohexane to afford the title product (228 mg).

LCMS (Method 1): Rt=1.35 min

¹H-NMR (300 MHz, DMSO-d₆) δ: 7.18 (d, J=2.5 Hz, 1H), 6.98 (d, J=8.6 Hz, 1H), 6.57 (dd, J=8.5, 2.6 Hz, 1H), 3.69 (s, 3H), 3.56-3.60 (m, 2H), 2.63 (t, J=6.46 Hz, 2H), 1.74-1.82 (m, 2H), 1.45 (s, 9H).

### Step 2

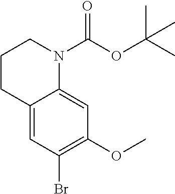 [SMILES: COc1cc2c(cc1Br)CCCN2C(=O)OC(C)(C)C]

### tert-Butyl 6-bromo-7-methoxy-3,4-dihydroquinoline-1(2H)-carboxylate (Intermediate 4)

A solution of intermediate 4-1 (228 mg, 0.86 mmol) in EtOAc (10 mL) was cooled to 0° C. 1,3-dibromo-5,5-dimethyl-imidazolidine-2,4-dione (124 mg, 43.3 mmol) was added portion wise over 15 min. RM was stirred at 0° C. for 20 min, then quenched with 10% (w/w) aq. K₂CO₃ (20 mL). The organic layer was separated, washed with sat. aq. NaCl (20 mL) and concentrated in vacuo. The residue was purified by flash chromatography on Si cartridge by eluting with 0-15% EtOAc in cyclohexane to afford the title product (240 mg).

LCMS (Method 1): Rt=1.46 min, ES⁺ m/z 285.9/287.9 [M+H]⁺.

### Intermediate 5

### Step 1

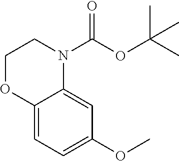 [SMILES: COc1ccc2c(c1)N(C(=O)OC(C)(C)C)CCO2]

### tert-butyl 6-methoxy-2,3-dihydro-4H-benzo[b][1,4]oxazine-4-carboxylate (Intermediate 5-1)

The title compound was prepared in a similar manner of intermediate 4-step 1 starting from 6-methoxy-3,4-dihydro-2H-1,4-benzoxazine.

LCMS (Method 5): Rt=2.5 min

¹H-NMR (300 MHz, DMSO-d₆) δ: 7.47-7.38 (m, 1H), 6.77 (d, J=8.9 Hz, 1H), 6.57 (dd, J=8.9, 3.0 Hz, 1H), 4.18-4.09 (m, 2H), 3.80-3.72 (m, 2H), 3.68 (s, 3H), 1.50 (s, 9H).

### Step 2

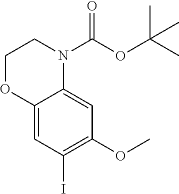 [SMILES: COc1cc2c(cc1I)OCCN2C(=O)OC(C)(C)C]

### tert-butyl 7-iodo-6-methoxy-2,3-dihydro-4H-benzo[b][1,4]oxazine-4-carboxylate (Intermediate 5)

Intermediate 5-step 1 (5.9 g, 22.1 mmol) was dissolved in DMF (60 mL), then N-Iodohydroxysuccinimide (12.7 g, 111 mmol) added and RM stirred at 40° C. overnight. RM was quenched in cold water and extracted with EtOAc. Combined organic layers were evaporated to dryness and the residue purified by chromatography on silica gel by gradient elution from 1:1 to 3:2 DCM-Hexane to afford the title compound (7.38 g).

LCMS (Method 1): Rt=2.8 min

¹H-NMR (300 MHz, DMSO-d₆) δ: 7.50 (s, 1H), 7.24 (s, 1H), 4.14 (dd, J=5.3, 3.8 Hz, 2H), 3.80-3.75 (m, 2H), 3.74 (s, 3H), 1.51 (s, 9H).

### Intermediate 6

### Step 1

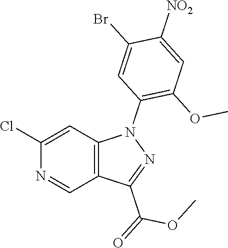 [SMILES: COC(=O)c1nn(-c2cc(Br)c([N+](=O)[O-])cc2OC)c2cc(Cl)ncc12]

### Methyl 1-(5-bromo-2-methoxy-4-nitrophenyl)-6-chloro-1H-pyrazolo[4,3-c]pyridine-3-carboxylate (Intermediate 6-1)

6-Chloro-1H-pyrazolo[4,3-c]pyridine-3-carboxylic acid (3.00 g, 15.2 mmol) was added to a solution of 1-bromo-5-fluoro-4-methoxy-2-nitro-benzene (3.80 g, 20.2 mmol) and DBU (5.88 mL, 45.6 mmol) in DMF (48 mL). RM was stirred at RT for 4 h. Iodomethane (3.78 mL, 60.7 mmol) was added and RM stirred at RT overnight. RM was quenched into water (200 mL), the formed precipitate was collected by filtration and dried. The crude material was triturated in EtOAc and washed with EtOAc to give the title product (5.83 g) that was used in the next synthetic step without further purification.

LCMS (Method 1): Rt=1.08 min, ES⁺ m/z 426.9/428.9/430.9 [M+H]⁺.

### Step 2

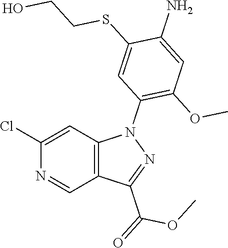 [SMILES: COC(=O)c1nn(-c2cc(SCCO)c(N)cc2OC)c2cc(Cl)ncc12]

### Methyl 6-chloro-1-(5-((2-hydroxyethyl)thio)-2-methoxy-4-nitrophenyl)-1H-pyrazolo[4,3-c]pyridine-3-carboxylate (Intermediate 6-2)

A degassed mixture of intermediate 6-1 (5.0 g, 11 mmol), 2-sulfanylethanol (1.13 mL, 12 mmol), DIPEA (5.91 mL, 20 mmol), Xantphos (820 mg, 1.4 mmol) and Pd₂(dba)₃ (330 mg, 0.57 mmol) in 1,4-dioxane (100 mL) was stirred at 100° C. under argon for 3 h. After cooling to RT, reaction was quenched with water. The formed precipitate was collected by filtration, washed with a small amount of EtOAc and dried to afford the title compound (5.3 g) that was used in the next synthetic step without further purification.

LCMS (Method 1): Rt=1.08 min, ES⁺ m/z 439.0/441.0 [M+H]⁺.

### Step 3

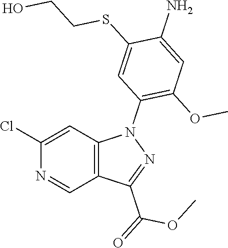 [SMILES: COC(=O)c1nn(-c2cc(SCCO)c(N)cc2OC)c2cc(Cl)ncc12]

### Methyl 1-(4-amino-5-((2-hydroxyethyl)thio)-2-methoxyphenyl)-6-chloro-1H-pyrazolo[4,3-c]pyridine-3-carboxylate (Intermediate 6-3)

A solution of intermediate 6-2 (5.3 g, 12 mmol) in MeOH (200 mL) was stirred at 80° C., then followed by the addition of a solution of NH₄Cl (6.5 g, 121 mmol) in water (20 mL) and iron powder (6.7 g, 121 mmol). RM was stirred at 80° C. for 4.5 h, then filtered while still hot and the filtrate concentrated in vacuo. The crude material was diluted with sat. aq NaHCO₃, the resulting suspension sonicated and the solids collected by filtration. The solid material was washed several times with water and dried to afford the title product (3.13 g).

LCMS (Method 1): Rt=0.92 min, ES⁺ m/z 409.1/411.1 [M+H]⁺.

### Step 4

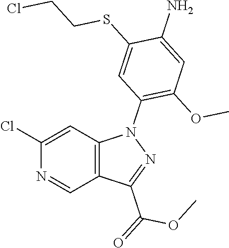 [SMILES: COC(=O)c1nn(-c2cc(SCCCl)c(N)cc2OC)c2cc(Cl)ncc12]

### Methyl 1-(4-amino-5-((2-chloroethyl)thio)-2-methoxyphenyl)-6-chloro-1H-pyrazolo[4,3-c]pyridine-3-carboxylate (Intermediate 6-4)

Intermediate 6-3 (4.4 g, 11 mmol) was mixed with thionyl chloride (12 mL, 165 mmol) at 0° C. RM was warmed to RT and stirred for 2 h, then volatiles removed in vacuo and the residue taken with sat. aq. NaHCO₃. The formed slurry was sonicated and solid collected by filtration, washed with water and dried. The resulting crude was purified by flash chromatography on Si cartridge by eluting with 0-10% EtOAc in DCM to afford the title product (2.7 g).

LCMS (Method 1): Rt=1.24 min, ES⁺ m/z 427.0/429.0/431.0 [M+H]⁺.

### Step 5

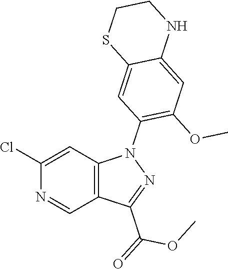 [SMILES: COC(=O)c1nn(-c2cc3c(cc2OC)NCCS3)c2cc(Cl)ncc12]

### Methyl 6-chloro-1-(6-methoxy-3,4-dihydro-2H-benzo[b][1,4]thiazin-7-yl)-1H-pyrazolo[4,3-c]pyridine-3-carboxylate (Intermediate 6-5)

A mixture of intermediate 6-4 (2.70 g, 6.32 mmol), K₂CO₃ (2.62 g, 19 mmol) and NaI (189 mg, 1.26 mmol) in DMF (80 mL) was stirred at 90° C. overnight. After cooling to RT, RM was diluted with water (200 mL). The formed precipitate was filtered, washed several times with water and dried. The resulting material was purified by flash chromatography on Si cartridge by eluting with 0-100% DCM/Acetonitrile (9:1) in DCM to afford the title product (730 mg).

LCMS (Method 1): Rt=1.13 min, ES⁺ m/z 391.1/393.1 [M+H]⁺.

### Step 6

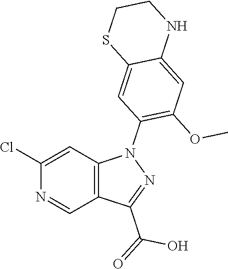 [SMILES: COc1cc2c(cc1-n1nc(C(=O)O)c3cnc(Cl)cc31)SCCN2]

### 6-Chloro-1-(6-methoxy-3,4-dihydro-2H-benzo[b][1,4]thiazin-7-yl)-1H-pyrazolo[4,3-c]pyridine-3-carboxylic acid (Intermediate 6)

Intermediate 6-5 (730 mg, 1.87 mmol) was suspended in THF (15 mL) and added with a solution of LiOH (1.0 M in water, 9 mL, 9 mmol). RM was stirred at 40° C. for 2.5 h. Organic solvent was removed in vacuo and the residue diluted with water. The pH was adjusted to 2.5 using aq. 1M HCl. The formed precipitate was filtered, washed several times with water and dried to afford the title compound (698 mg).

LCMS (Method 1): Rt=0.95 min, ES⁺ m/z 377.1/379.0 [M+H]⁺.

### Intermediate 7

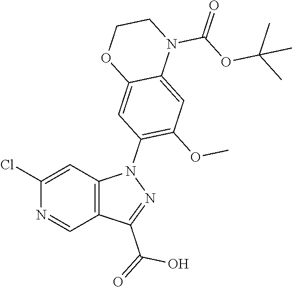 [SMILES: COc1cc2c(cc1-n1nc(C(=O)O)c3cnc(Cl)cc31)OCCN2C(=O)OC(C)(C)C]

### 1-(4-(tert-Butoxycarbonyl)-6-methoxy-3,4-dihydro-2H-benzo[b][1,4]oxazin-7-yl)-6-chloro-1H-pyrazolo[4,3-c]pyridine-3-carboxylic acid (Intermediate 7)

### Method 1

A degassed mixture of 6-Chloro-1H-pyrazolo[4,3-c]pyridine-3-carboxylic acid (400 mg, 2.02 mmol), intermediate 3 (733 mg, 2.13 mmol), Cs₂CO₃ (2.31 g, 7.09 mmol) and thiophene-2-carbonyloxycopper (278 mg, 1.46 mmol) in anhydrous DMSO (7 mL) was stirred under nitrogen at 110° C. overnight. After cooling to RT, RM was filtered. The filtrate was diluted with MeCN (50 mL) thus the formed precipitate collected by filtration and triturated with MeOH. The crude material was purified by flash chromatography on Si cartridge by eluting with 0-100% MeOH in EtOAc to afford the title product (150 mg).

### Method 2

A degassed mixture of 6-Chloro-1H-pyrazolo[4,3-c]pyridine-3-carboxylic acid (9.4 g, 47.5 mmol), intermediate 5 (18.6 g, 47.5 mmol), Cs₂CO₃ (54 g, 166.3 mmol) and thiophene-2-carbonyloxycopper (9 g, 47.5 mmol) in DMSO (100 mL) was stirred under argon at 110° C. for 24 h. RM was quenched in water and extracted with DCM. Combined organic layers was washed with aq 10% w/w citric acid, aq. sat. NaCl and evaporated to dryness. The residue was purified by chromatography on silica gel by eluting with DCM to DCM (1% v/v MeOH+1% AcOH v/v). The material thus obtained was triturated in ethyl ether to afford the title compound (3.14 g).

LCMS (Method 1): Rt=1.22 min, ES⁺ m/z 461.2/463.2 [M+H]⁺.

### Intermediate 8

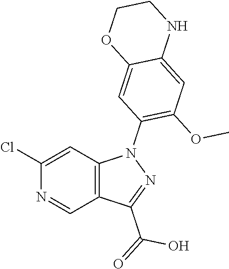 [SMILES: COc1cc2c(cc1-n1nc(C(=O)O)c3cnc(Cl)cc31)OCCN2]

### 6-Chloro-1-(6-methoxy-3,4-dihydro-2H-benzo[b][1,4]oxazin-7-yl)-1H-pyrazolo[4,3-c]pyridine-3-carboxylic acid (Intermediate 8

Intermediate 8 was prepared in a similar manner to Intermediate 7 (Method 1) starting from 6-Chloro-1H-pyrazolo[4,3-c]pyridine-3-carboxylic acid (200 mg, 1.0 mmol) and intermediate 3-1 (371 mg, 1.5 mmol).

LCMS (Method 1): Rt=0.86 min, ES⁺ m/z 360.9/362.8 [M+H]⁺.

### Intermediate 9

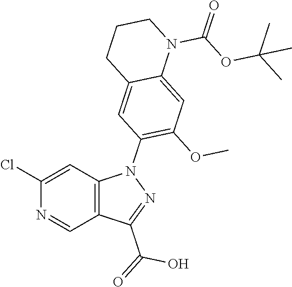 [SMILES: COc1cc2c(cc1-n1nc(C(=O)O)c3cnc(Cl)cc31)CCCN2C(=O)OC(C)(C)C]

### 1-(1-(tert-Butoxycarbonyl)-7-methoxy-1,2,3,4-tetrahydroquinolin-6-yl)-6-chloro-1H-pyrazolo[4,3-c]pyridine-3-carboxylic acid (Intermediate 9)

A mixture of intermediate 4 (1.04 g, 3.04 mmol), 6-Chloro-1H-pyrazolo[4,3-c]pyridine-3-carboxylic acid (300 mg, 1.52 mmol), thiophene-2-carbonyloxycopper (290 mg, 1.52 mmol) and Cs₂CO₃ (1.48 g, 4.56 mmol) in DMSO (7 mL) was stirred for 72 h at 100° C. under nitrogen. After cooling to RT, RM was added dropwise to water under stirring. The pH was adjusted to 4 (with aq. HCl 1M) and the formed precipitate filtered, washed with water, and dried. The crude material was purified by flash chromatography on Si cartridge by eluting with 0-50% DCM:MeOH:HCO₂H (90:10:0.3) in DCM to afford the title product (330 mg).

LCMS (Method 1): Rt=1.24 min, ES⁺ m/z 458.9/460.9 [M+H]⁺.

### Intermediate 10

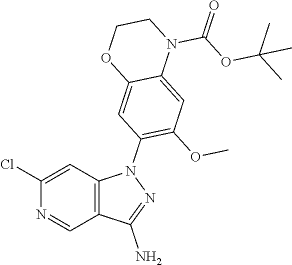 [SMILES: COc1cc2c(cc1-n1nc(N)c3cnc(Cl)cc31)OCCN2C(=O)OC(C)(C)C]

### tert-Butyl 7-(3-amino-6-chloro-1H-pyrazolo[4,3-c]pyridin-1-yl)-6-methoxy-2,3-dihydro-4H-benzo[b][1,4]oxazine-4-carboxylate (Intermediate 10)

A degassed mixture of intermediate 3 (408 mg, 1.19 mmol), 6-chloro-1H-pyrazolo[4,3-c]pyridin-3-amine (200 mg, 1.19 mmol), Cs₂CO₃ (980 mg, 3.01 mmol), DMCHDA (93.5 μL, 0.59 mmol) and CuI (113 mg, 0.59 mmol) in DMSO (4.9 mL) was stirred under argon at 110° C. overnight. After cooling to RT, RM was quenched with sat. aq. NaHCO₃ (50 mL) and extracted with EtOAc (5×50 mL). Combined organic layers were washed with sat. aq. NaCl, dried over Na₂SO₄ and solvent removed in vacuo. The crude residue was purified by flash chromatography on Si cartridge by eluting with 0-50% DCM/MeOH/NH₄OH (90:5:0.5) in DCM to afford the title product (282 mg).

LCMS (Method 1): Rt=1.15 min, ES⁺ m/z 431.9/433.9 [M+H]⁺.

### Intermediate 11a

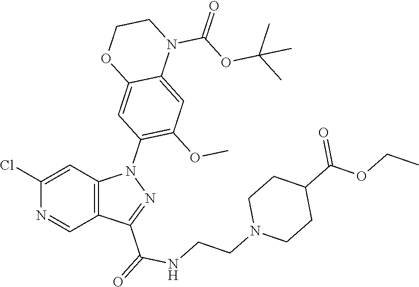 [SMILES: CCOC(=O)C1CCN(CCNC(=O)c2nn(-c3cc4c(cc3OC)N(C(=O)OC(C)(C)C)CCO4)c3cc(Cl)ncc23)CC1]

### tert-Butyl 7-(6-chloro-3-((2-(4-(ethoxycarbonyl)piperidin-1-yl)ethyl)carbamoyl)-1H-pyrazolo[4,3-c]pyridin-1-yl)-6-methoxy-2,3-dihydro-4H-benzo[b][1,4]oxazine-4-carboxylate (Intermediate 11a)

A mixture of intermediate 7 (700.0 mg, 1.52 mmol), intermediate 1 (369 mg, 1.82 mmol), DIPEA (1.06 mL, 6.08 mmol), HATU (693 mg, 1.82 mmol) and DMF (12 mL) was stirred at RT for 2.5 h. RM was diluted with water (40 mL) and extracted with EtOAc (2×30 mL). Combined organic layers were washed with sat. aq. NaCl (30 mL) and solvent removed in vacuo. The residue was purified by flash chromatography on Si cartridge by eluting with 0-60% DCM/EtOH/NH₄OH (90:5:0.5) in DCM to afford the title product (866 mg).

LCMS (Method 1): Rt=1.02 min, ES⁺ m/z 643.4/645.4 [M+H]⁺.

### Intermediates 11b to 11e

The following intermediates were prepared in a similar manner to Intermediate 11a from the indicated starting materials.

| | | Starting | |
|---|---|---|---|
| Intermediate | Structure | materials | LCMS |
| Intermediate 11b | 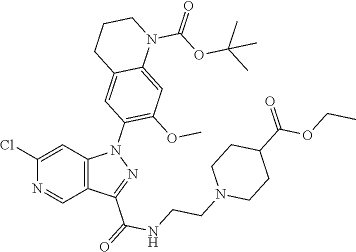 [SMILES: CCOC(=O)C1CCN(CCNC(=O)c2nn(-c3cc4c(cc3OC)N(C(=O)OC(C)(C)C)CCC4)c3cc(Cl)ncc23)CC1] | Int 1 and Int 9 | (Method 1): Rt = 1.02 min, ES⁺ m/z 641.1/643.1 [M + H]⁺ |
| Intermediate 11c | 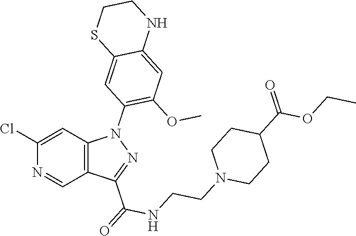 [SMILES: CCOC(=O)C1CCN(CCNC(=O)c2nn(-c3cc4c(cc3OC)NCCS4)c3cc(Cl)ncc23)CC1] | Int 1 and Int 6 | (Method 1): Rt = 1.85 min, ES⁺ m/z 559.2/561.2 [M + H]⁺ |
| Intermediate 11d | 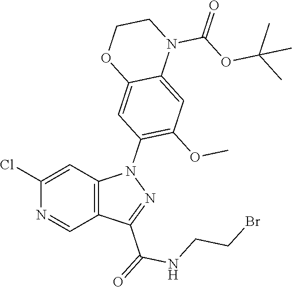 [SMILES: COc1cc2c(cc1-n1nc(C(=O)NCCBr)c3cnc(Cl)cc31)OCCN2C(=O)OC(C)(C)C] | 2-bromo- ethylamine hydrochloride and Int 7 | (Method 2): Rt = 1.38 min, ES⁺ m/z 566.6/568.1/570.1 [M + H]⁺ |
| Intermediate 11e | 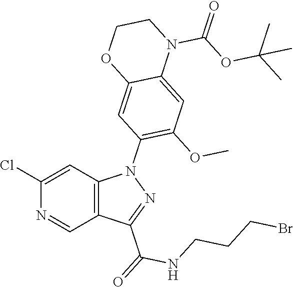 [SMILES: COc1cc2c(cc1-n1nc(C(=O)NCCCBr)c3cnc(Cl)cc31)OCCN2C(=O)OC(C)(C)C] | 3-bromo- propan-1-amine hydrochloride and Int 7 | (Method 6): Rt = 1.36 min, ES⁺ m/z 582.2/580.2/584.1 [M + H]⁺ |

### Intermediate 12

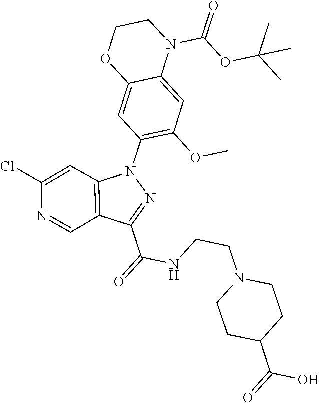 [SMILES: COc1cc2c(cc1-n1nc(C(=O)NCCN3CCC(C(=O)O)CC3)c3cnc(Cl)cc31)OCCN2C(=O)OC(C)(C)C]

### 1-(2-(1-(4-(tert-butoxycarbonyl)-6-methoxy-3,4-dihydro-2H-benzo[b][1,4]oxazin-7-yl)-6-chloro-1H-pyrazolo[4,3-c]pyridine-3-carboxamido)ethyl)piperidine-4-carboxylic acid (Intermediate 12)

To a solution of intermediate 11a (1.0 g, 1.48 mmol) in THF (10 mL), an aqueous 1M solution of LiOH (10.6 mL, 11.8 mmol) was added at RT. RM was stirred at RT for 16 h. Volatiles were removed in vacuo and the residue diluted with water. The pH was adjusted to 6.5 using 1M aq. HCl and RM extracted with DCM (3×10 mL). Combined organic layers were evaporated to dryness to give the title product (756 mg).

LCMS (Method 1): Rt=0.99 min, ES⁺ m/z 615.1/617.1 [M+H]⁺

### Intermediate 13a

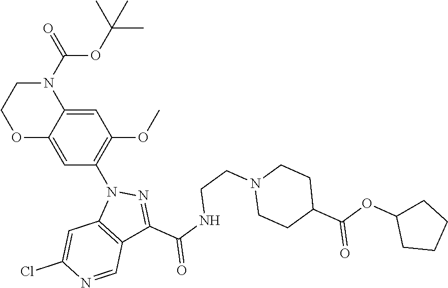 [SMILES: COc1cc2c(cc1-n1nc(C(=O)NCCN3CCC(C(=O)OC4CCCC4)CC3)c3cnc(Cl)cc31)OCCN2C(=O)OC(C)(C)C]

### tert-Butyl 7-(6-chloro-3-((2-(4-((cyclopentyloxy)carbonyl)piperidin-1-yl)ethyl)carbamoyl)-1H-pyrazolo[4,3-c]pyridin-1-yl)-6-methoxy-2,3-dihydro-4H-benzo[b][1,4]oxazine-4-carboxylate (Intermediate 13a)

A solution of intermediate 12 (80.0 mg, 0.13 mmol), cyclopentanol (102 μL, 0.77 mmol) EDC*HCl (24.7 mg, 0.13 mmol) and DMAP (6.29 mg, 0.05 mmol) in dry DCM (3 mL) was stirred under nitrogen at RT for 3 h. RM was diluted with water (10 mL) and extracted with DCM (2×10 mL). Combined organics were washed with sat. aq. NaCl (10 mL) and solvent removed in vacuo. The residue was purified by flash chromatography on Si cartridge by eluting with 0-90% DCM/MeOH/NH₄OH (90:9:1.5) in DCM to afford the title product (55 mg).

LCMS (Method 2), Rt=1.54, ES⁺ m/z 683.4/685.4.

### Intermediates 13b to 13o

The following intermediates were prepared in a similar manner to intermediate 13a from the indicated starting materials.

| | | Starting | |
|---|---|---|---|
| Intermediate | Structure | materials | LCMS |
| Intermediate 13b | 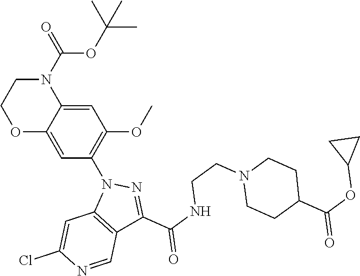 [SMILES: COc1cc2c(cc1-n1nc(C(=O)NCCN3CCC(C(=O)OC4CC4)CC3)c3cnc(Cl)cc31)OCCN2C(=O)OC(C)(C)C] | Int 12 and cyclo- propanol | (Method 1): Rt = 1.00 min, ES⁺ m/z 655.4/657.4 [M + H]⁺ |
| Intermediate 13c | 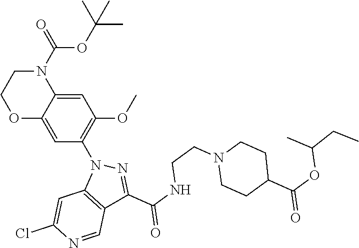 [SMILES: CCC(C)OC(=O)C1CCN(CCNC(=O)c2nn(-c3cc4c(cc3OC)N(C(=O)OC(C)(C)C)CCO4)c3cc(Cl)ncc23)CC1] | Int 12 and sec-butanol | (Method 2): Rt = 1.52 min, ES⁺ m/z 671.4/673.3 [M + H]⁺ |
| Intermediate 13d | 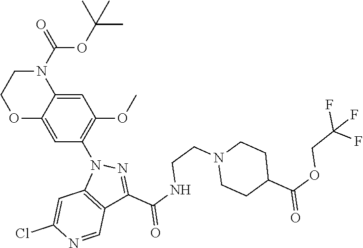 [SMILES: COc1cc2c(cc1-n1nc(C(=O)NCCN3CCC(C(=O)OCC(F)(F)F)CC3)c3cnc(Cl)cc31)OCCN2C(=O)OC(C)(C)C] | Int 12 and 2,2,2- trifluoro- ethanol | (Method 1): Rt = 1.01 min, ES⁺ m/z 697.3/699.3 [M + H]⁺ |
| Intermediate 13e | 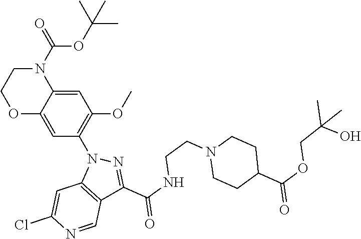 [SMILES: COc1cc2c(cc1-n1nc(C(=O)NCCN3CCC(C(=O)OCC(C)(C)O)CC3)c3cnc(Cl)cc31)OCCN2C(=O)OC(C)(C)C] | Int 12 and 2-methyl- propane- 1,2-diol | (Method 1): Rt = 0.94 min, ES⁺ m/z 687.2/689.2 [M + H]⁺ |
| Intermediate 13f | 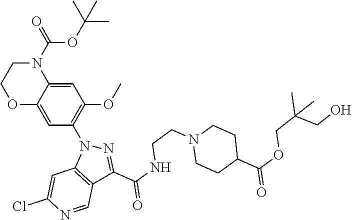 [SMILES: COc1cc2c(cc1-n1nc(C(=O)NCCN3CCC(C(=O)OCC(C)(C)CO)CC3)c3cnc(Cl)cc31)OCCN2C(=O)OC(C)(C)C] | Int 12 and 2,2-dimethyl- propane- 1,3-diol | (Method 1): Rt = 0.99 min, ES⁺ m/z 701.4/703.4 [M + H]⁺ |
| Intermediate 13g | 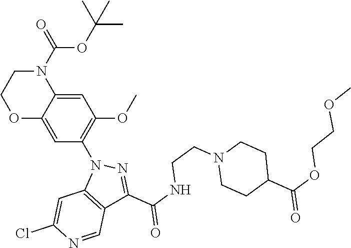 [SMILES: COCCOC(=O)C1CCN(CCNC(=O)c2nn(-c3cc4c(cc3OC)N(C(=O)OC(C)(C)C)CCO4)c3cc(Cl)ncc23)CC1] | Int 12 and 2-methoxy- ethanol | (Method 2): Rt = 1.33 min, ES⁺ m/z 673.4/675.4 [M + H]⁺ |
| Intermediate 13h | 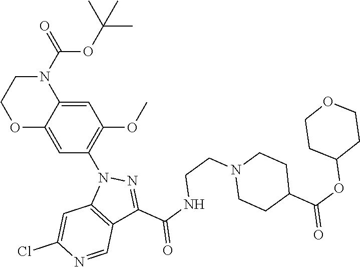 [SMILES: COc1cc2c(cc1-n1nc(C(=O)NCCN3CCC(C(=O)OC4CCOCC4)CC3)c3cnc(Cl)cc31)OCCN2C(=O)OC(C)(C)C] | Int 12 and tetrahydro- pyran-4-ol | (Method 1): Rt = 0.99 min, ES⁺ m/z 699.3/701.2 [M + H]⁺ |
| Intermediate 13i | 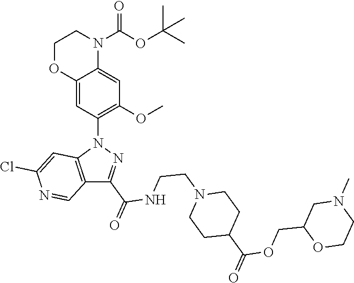 [SMILES: COc1cc2c(cc1-n1nc(C(=O)NCCN3CCC(C(=O)OCC4CN(C)CCO4)CC3)c3cnc(Cl)cc31)OCCN2C(=O)OC(C)(C)C] | Int 12 and (4-methyl- morpholin- 2-yl)- methanol | (Method 1): Rt = 0.83 min, ES⁺ m/z 728.3/730.2 [M + H]⁺ |
| Intermediate 13j | 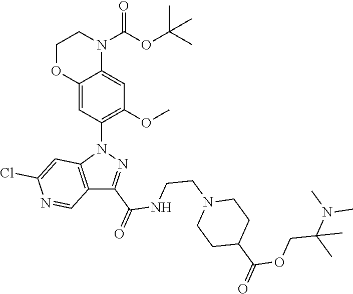 [SMILES: COc1cc2c(cc1-n1nc(C(=O)NCCN3CCC(C(=O)OCC(C)(C)N(C)C)CC3)c3cnc(Cl)cc31)OCCN2C(=O)OC(C)(C)C] | Int 12 and 2- (dimethyl- amino)-2- methyl- propan-1-ol | (Method 1): Rt = 0.85 min, ES⁺ m/z 714.4/716.4 [M + H]⁺ |
| Intermediate 13k | 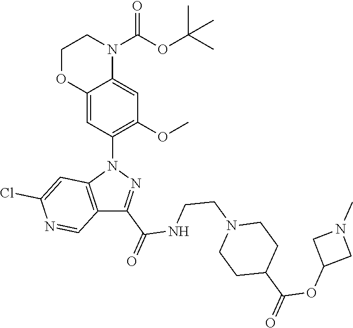 [SMILES: COc1cc2c(cc1-n1nc(C(=O)NCCN3CCC(C(=O)OC4CN(C)C4)CC3)c3cnc(Cl)cc31)OCCN2C(=O)OC(C)(C)C] | Int 12 and 1-methyl- azetidin- 3-ol hydro- chloride | (Method 1): Rt = 0.80 min, ES⁺ m/z 684.4/686.4 [M + H]⁺ |
| Intermediate 13l | 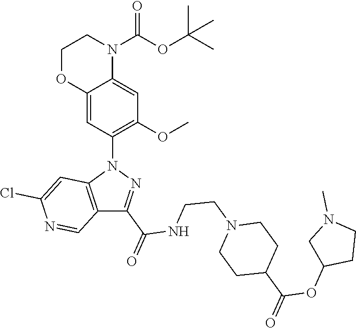 [SMILES: COc1cc2c(cc1-n1nc(C(=O)NCCN3CCC(C(=O)OC4CCN(C)C4)CC3)c3cnc(Cl)cc31)OCCN2C(=O)OC(C)(C)C] | Int 12 and 1-methyl- pyrrolidin- 3-ol | (Method 1): Rt = 0.82 min, ES⁺ m/z 698.4/700.4 [M + H]⁺ |
| Intermediate 13m | 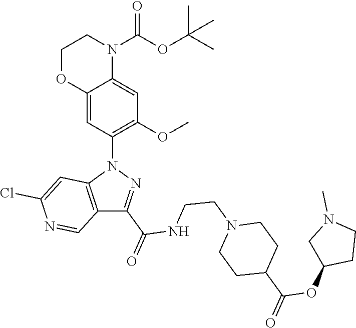 [SMILES: COc1cc2c(cc1-n1nc(C(=O)NCCN3CCC(C(=O)O[C@@H]4CCN(C)C4)CC3)c3cnc(Cl)cc31)OCCN2C(=O)OC(C)(C)C] | Int 12 and (R)-1-methyl- pyrrolidin- 3-ol | (Method 1): Rt = 1.04 min, ES⁺ m/z 698.4/700.4 [M + H]⁺ |
| Intermediate 13n | 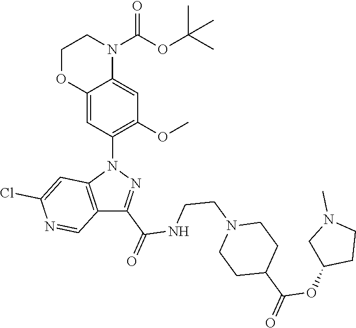 [SMILES: COc1cc2c(cc1-n1nc(C(=O)NCCN3CCC(C(=O)O[C@H]4CCN(C)C4)CC3)c3cnc(Cl)cc31)OCCN2C(=O)OC(C)(C)C] | Int 12 and (S)-1- methyl- pyrrolidin- 3-ol | (Method 1): Rt = 0.79 min, ES⁺ m/z 698.3/700.3 [M + H]⁺ |
| Intermediate 13o | 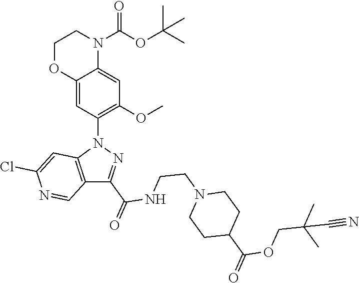 [SMILES: COc1cc2c(cc1-n1nc(C(=O)NCCN3CCC(C(=O)OCC(C)(C)C#N)CC3)c3cnc(Cl)cc31)OCCN2C(=O)OC(C)(C)C] | Int 12 and 3-hydroxy- 2,2-dimeth- yl-propane- nitrile | (Method 1): Rt = 1.05 min, ES⁺ m/z 696.2/698.2 [M + H]⁺ |

### Intermediate 13r

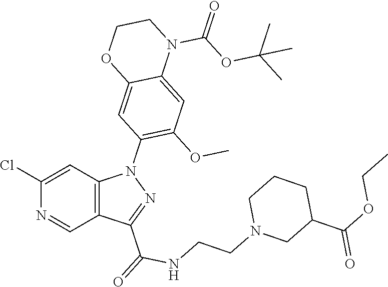 [SMILES: CCOC(=O)C1CCCN(CCNC(=O)c2nn(-c3cc4c(cc3OC)N(C(=O)OC(C)(C)C)CCO4)c3cc(Cl)ncc23)C1]

### tert-Butyl 7-(6-chloro-3-((2-(3-(ethoxycarbonyl)piperidin-1-yl)ethyl)carbamoyl)-1H-pyrazolo[4,3-c]pyridin-1-yl)-6-methoxy-2,3-dihydro-4H-benzo[b][1,4]oxazine-4-carboxylate (Intermediate 13r)

A solution of intermediate 11d (60 mg, 0.10 mmol), ethyl piperidine-3-carboxylate (16 mg, 0.10 mmol) and DIPEA (71 μL, 0.41 mmol) in DMF (1 mL) was stirred at RT for 6h. Ethyl piperidine-3-carboxylate (16 mg, 0.10 mmol) was added and RM stirred at RT overnight. RM was diluted with EtOAc (10 mL), washed with water (3×10 mL) and sat. aq. NaCl (10 mL). Organic layer was evaporated to dryness and the residue purified by flash chromatography on Si cartridge by eluting with 0-50% DCM/EtOH/NH₄OH (90:5:0.5) in DCM to afford the title product (30.1 mg).

LCMS (Method 1), Rt=1.05, ES⁺ m/z 643.3/645.4.

### Intermediates 13s-13aa

The following intermediates were prepared in a similar manner to intermediate 13r from the indicated starting materials. It is additionally stated if base, solvent, additive or temperature were varied.

| Intermediate | Structure | Starting material | LCMS |
|---|---|---|---|
| Intermediate 13s | 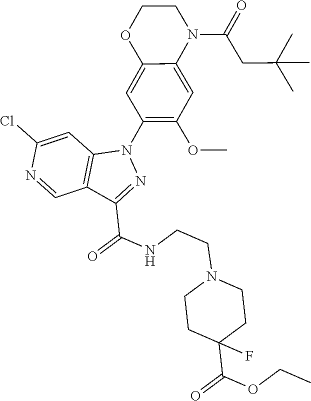 [SMILES: CCOC(=O)C1(F)CCN(CCNC(=O)c2nn(-c3cc4c(cc3OC)N(C(=O)CC(C)(C)C)CCO4)c3cc(Cl)ncc23)CC1] | Int 11d and ethyl 4- fluoropiperidine-4- carboxylate hydro- chloride Solvent: acetone, base: Na₂CO₃ (5 eq.) additive: NaI (0.2 eq.) | (Method 1): Rt = 1.01 min, ES⁺ m/z 661.3/663.2 [M + H]⁺ |
| Intermediate 13t | 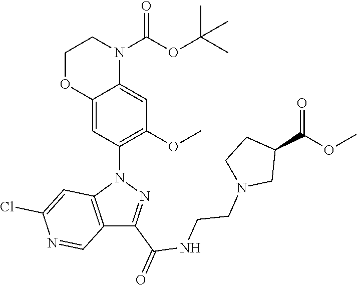 [SMILES: COC(=O)[C@@H]1CCN(CCNC(=O)c2nn(-c3cc4c(cc3OC)N(C(=O)OC(C)(C)C)CCO4)c3cc(Cl)ncc23)C1] | Int 11d and methyl (3R)-pyrrolidine-3- carboxylate hydro- chloride | (Method 1): Rt = 0.97 min, ES⁺ m/z 615.3/617.2 [M + H]⁺ |
| Intermediate 13u | 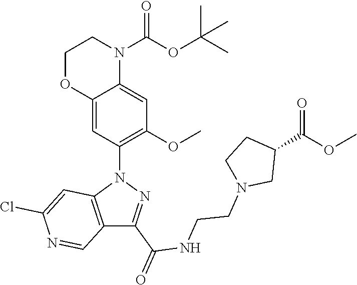 [SMILES: COC(=O)[C@H]1CCN(CCNC(=O)c2nn(-c3cc4c(cc3OC)N(C(=O)OC(C)(C)C)CCO4)c3cc(Cl)ncc23)C1] | Int 11d and methyl (3S)-pyrrolidine-3- carboxylate hydro- chloride | (Method 1): Rt = 0.98 min, ES⁺ m/z 615.3/617.2 [M + H]⁺ |
| Intermediate 13v | 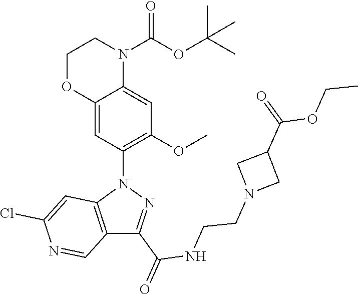 [SMILES: CCOC(=O)C1CN(CCNC(=O)c2nn(-c3cc4c(cc3OC)N(C(=O)OC(C)(C)C)CCO4)c3cc(Cl)ncc23)C1] | Int 11d and ethyl azetidine-3-carbox- ylate hydrochloride | (Method 1): Rt = 1.01 min, ES⁺ m/z 615.3/617.2 [M + H]⁺ |
| Intermediate 13w | 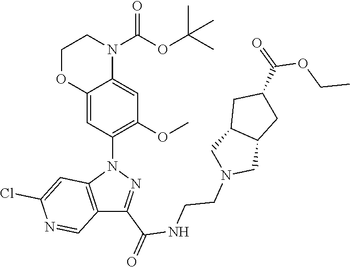 [SMILES: CCOC(=O)[C@@H]1C[C@@H]2CN(CCNC(=O)c3nn(-c4cc5c(cc4OC)N(C(=O)OC(C)(C)C)CCO5)c4cc(Cl)ncc34)C[C@@H]2C1] | Int 11d and ethyl (3aS,6aR)-1,2,3,3a, 4,5,6,6a-octahydro- cyclopenta[c]pyrrole- 5-carboxylate | (Method 1): Rt = 1.06 min, ES⁺ m/z 669.4/671.4 [M + H]⁺ |
| Intermediate 13x | 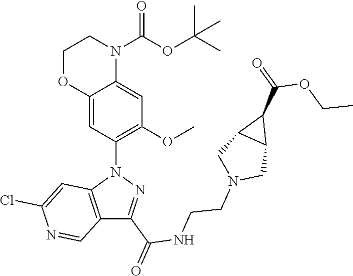 [SMILES: CCOC(=O)[C@H]1[C@@H]2CN(CCNC(=O)c3nn(-c4cc5c(cc4OC)N(C(=O)OC(C)(C)C)CCO5)c4cc(Cl)ncc34)C[C@@H]21] | Int 11d and ethyl (1R,5S)-3-azabicyclo [3.1.0]hexane-6- carboxylate | (Method 1): Rt = 1.02 min, ES⁺ m/z 641.4/643.4 [M + H]⁺ |
| Intermediate 13y | 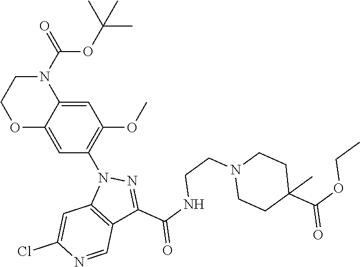 [SMILES: CCOC(=O)C1(C)CCN(CCNC(=O)c2nn(-c3cc4c(cc3OC)N(C(=O)OC(C)(C)C)CCO4)c3cc(Cl)ncc23)CC1] | Int 11d and ethyl 4- methylpiperidine-4- carboxylate | (Method 1): Rt = 1.18 min, ES⁺ m/z 657.3/659.2 [M + H]⁺ |
| Intermediate 13z | 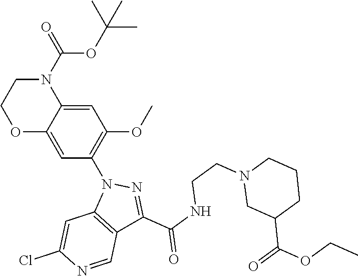 | Int 11d and ethyl 2- (4-piperidyl)acetate hydrochloride | (Method 1): Rt = 1.01 min, ES⁺ m/z 657.3/659.3 [M + H]⁺ |
| Intermediate 13aa | 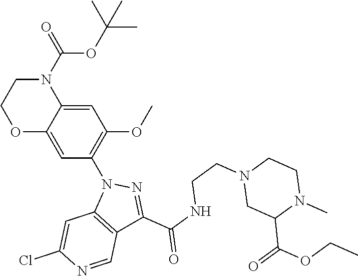 [SMILES: CCOC(=O)C1CN(CCNC(=O)c2nn(-c3cc4c(cc3OC)N(C(=O)OC(C)(C)C)CCO4)c3cc(Cl)ncc23)CCN1C] | Int 11d and ethyl 2- (4-piperidyl)acetate hydrochloride | (Method 1): Rt = 1.01 min, ES⁺ m/z 658.3/660.3 [M + H]⁺ |

### Intermediate 13ab

### Step 1

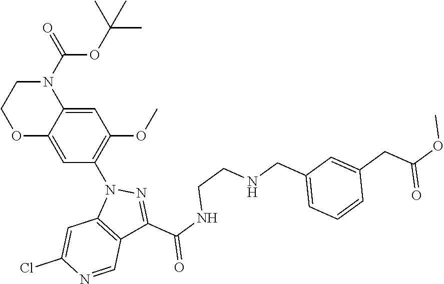 [SMILES: COC(=O)Cc1cccc(CNCCNC(=O)c2nn(-c3cc4c(cc3OC)N(C(=O)OC(C)(C)C)CCO4)c3cc(Cl)ncc23)c1]

### tert-Butyl 7-(6-chloro-3-((2-((3-(2-methoxy-2-oxoethyl)benzyl)amino)ethyl)carbamoyl)-1H-pyrazolo[4,3-c]pyridin-1-yl)-6-methoxy-2,3-dihydro-4H-benzo[b][1,4]oxazine-4-carboxylate (Intermediate 13ab-1)

The title compound was prepared in a similar manner of intermediate 13r starting with Intermediate 11d and methyl 2-[3-(aminomethyl)phenyl]acetate hydrochloride.

LCMS (Method 1): Rt=1.06 min, ES⁺ m/z 665.3/667.3 [M+H]⁺

### Step 2

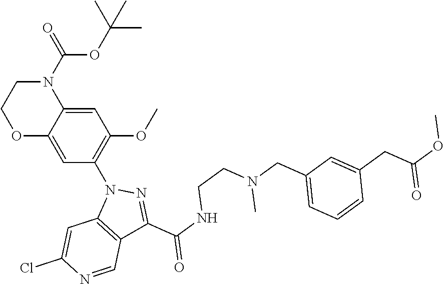 [SMILES: COC(=O)Cc1cccc(CN(C)CCNC(=O)c2nn(-c3cc4c(cc3OC)N(C(=O)OC(C)(C)C)CCO4)c3cc(Cl)ncc23)c1]

### tert-Butyl 7-(6-chloro-3-((2-((3-(2-methoxy-2-oxoethyl)benzyl)(methyl)amino)ethyl)carbamoyl)-1H-pyrazolo[4,3-c]pyridin-1-yl)-6-methoxy-2,3-dihydro-4H-benzo[b][1,4]oxazine-4-carboxylate (Intermediate 13ab)

A solution of intermediate 13ab-1 (64 mg, 0.10 mmol) in MeOH (1 mL) was treated with formic acid (145 μL, 3.8 mmol) and 37% aq. formaldehyde (287 μL, 3.8 mmol). RM was stirred at RT for 60 h. RM was diluted with EtOAc, washed with sat. aq. NaHCO₃ (3×15 mL) and sat. aq. NaCl (15 mL). The organic layer was evaporated in vacuo and the residue purified by flash chromatography on Si cartridge by eluting with 0-45% DCM/MeOH/NH₄OH (90:4:1) in DCM to afford the title product (28 mg).

LCMS (Method 2), Rt=1.46, ES⁺ m/z 679.2/681.2.

### Intermediate 13ac

### Step 1

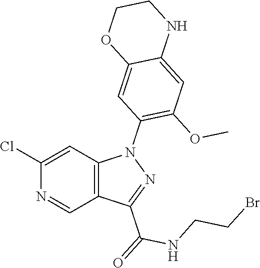 [SMILES: COc1cc2c(cc1-n1nc(C(=O)NCCBr)c3cnc(Cl)cc31)OCCN2]

### N-(2-Bromoethyl)-6-chloro-1-(6-methoxy-3,4-dihydro-2H-benzo[b][1,4]oxazin-7-yl)-1H-pyrazolo[4,3-c]pyridine-3-carboxamide (Intermediate 13ac-1)

A solution of intermediate 11d (100.0 mg, 0.17 mmol) and TFA (642.0 μL, 8.64 mmol) in DCM (1.0 mL) was stirred at RT for 16 h, then evaporated to dryness. The residue was dissolved in MeOH (0.5 mL) and passed through a SCX cartridge, washed with MeOH (20 mL) and eluted with 1M methanolic ammonia (20 mL) to afford the desired product (81.9 mg).

LCMS (Method 1): Rt=1.07, ES⁺ m/z 466.1/468.1.

### Step 2

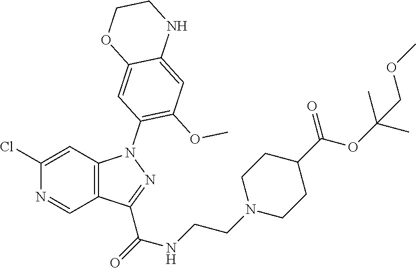 [SMILES: COCC(C)(C)OC(=O)C1CCN(CCNC(=O)c2nn(-c3cc4c(cc3OC)NCCO4)c3cc(Cl)ncc23)CC1]

### 1-Methoxy-2-methylpropan-2-yl 1-(2-(6-chloro-1-(6-methoxy-3,4-dihydro-2H-benzo[b][1,4]oxazin-7-yl)-1H-pyrazolo[4,3-c]pyridine-3-carboxamido)ethyl)piperidine-4-carboxylate (Intermediate 13ac)

The title compound was prepared in a similar manner of intermediate 13r starting with intermediate 13ac-1 and intermediate 2a.

LCMS (Method 1): Rt=0.85 min, ES⁺ m/z 601.4/603.4 [M+H]⁺

### Intermediate 13ad

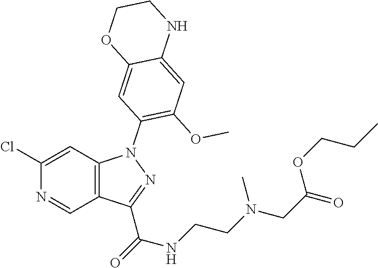 [SMILES: CCCOC(=O)CN(C)CCNC(=O)c1nn(-c2cc3c(cc2OC)NCCO3)c2cc(Cl)ncc12]

### Propyl N-(2-(6-chloro-1-(6-methoxy-3,4-dihydro-2H-benzo[b][1,4]oxazin-7-yl)-1H-pyrazolo[4,3-c]pyridine-3-carboxamido)ethyl)-N-methylglycinate (Intermediate 13ad)

A mixture of intermediate 11d (100 mg, 0.20 mmol), propyl 2-(methylamino)acetate (80.0 mg, 0.61 mmol), Na₂CO₃ (43.0 mg, 0.41 mmol) in acetone (2.0 mL) was stirred at 40° C. for 16 h and at 65° C. for 1 h. After cooling to RT, solvent was removed in vacuo. The residue was partitioned between EtOAc (10 mL) and water (10 mL), aqueous layer further extracted with EtOAc (2×10 mL). Combined organic layers were washed with sat. aq. NaHCO₃ (2×10 mL), with sat. aq. NaCl (10 mL) and evaporated in vacuo. The residue was purified by flash chromatography on Si cartridge by eluting with 0-60% DCM/MeOH (20:1) in DCM to afford the title product (51.7 mg).

LCMS (Method 2): Rt=1.11 min, ES⁺ m/z 517.2/519.2 [M+H]⁺

### Intermediate 13ae

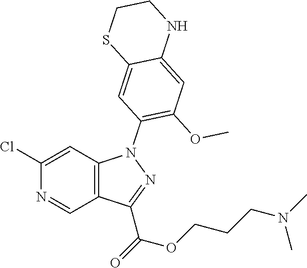 [SMILES: COc1cc2c(cc1-n1nc(C(=O)OCCCN(C)C)c3cnc(Cl)cc31)SCCN2]

### 3-(Dimethylamino)propyl 6-chloro-1-(6-methoxy-3,4-dihydro-2H-benzo[b][1,4]thiazin-7-yl)-1H-pyrazolo[4,3-c]pyridine-3-carboxylate (Intermediate 13ae)

A mixture of intermediate 6 (50.0 mg, 0.13 mmol), T3P® (50.0% solution in DMF, 155 μL, 0.26 mmol), azido(trimethyl)silane (35.2 μL, 0.26 mmol), TEA (55.5 μL, 0.4 mmol) and 3-(dimethylamino)propan-1-ol (31.4 μL, 0.26 mmol) in THF (1 mL) was refluxed for 3 h. Reaction mixture was refluxed for 3 h., diluted with EtOAc (25 mL), washed with sat. aq. NaHCO₃ (3×15 mL) and sat. aq. NaCl (15 mL). Organic layer was dried over Na₂SO₄ and concentrated in vacuo. The residue was purified by flash chromatography on Si cartridge by eluting with 0-35% DCM/MeOH/NH₄OH (90:15:1.5) in DCM to afford the desired product.

LCMS (Method 1): Rt=0.83 min, ES⁺ m/z 462.1/464.2 [M+H]⁺

### Intermediate 13af

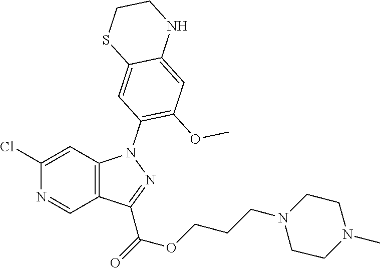 [SMILES: COc1cc2c(cc1-n1nc(C(=O)OCCCN3CCN(C)CC3)c3cnc(Cl)cc31)SCCN2]

### 3-(4-Methylpiperazin-1-yl)propyl 6-chloro-1-(6-methoxy-3,4-dihydro-2H-benzo[b][1,4]thiazin-7-yl)-1H-pyrazolo[4,3-c]pyridine-3-carboxylate (Intermediate 13af)

The title product was prepared on a similar manner to intermediate 13ae from intermediate 6 (60 mg, 0.16 mmol) and 3-(4-methylpiperazin-1-yl)propan-1-ol (50 mg, 0.32 mmol).

LCMS (Method 1): Rt=0.81 min, ES⁺ m/z 517.1/519.1 [M+H]⁺

### Intermediates 13ag to 13ai

The following intermediates were prepared in a similar manner to intermediate 13r from the indicated starting materials.

| | | Starting | |
|---|---|---|---|
| Intermediate | Structure | materials | LCMS |
| Intermediate 13ag | 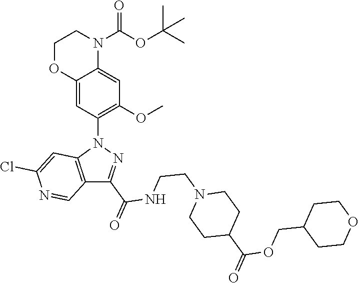 [SMILES: COc1cc2c(cc1-n1nc(C(=O)NCCN3CCC(C(=O)OCC4CCOCC4)CC3)c3cnc(Cl)cc31)OCCN2C(=O)OC(C)(C)C] | Int 11d and Int 2b | (Method 6): Rt = 1.26 min, ES⁺ m/z 713.4/715.4 [M + H]⁺ |
| Intermediate 13ah | 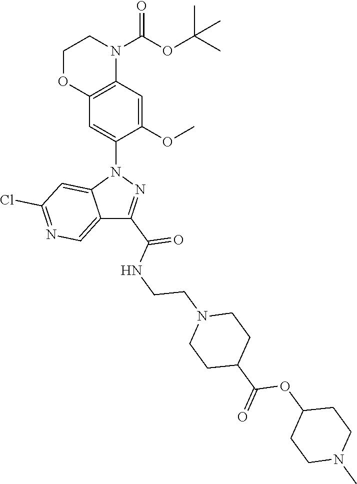 [SMILES: COc1cc2c(cc1-n1nc(C(=O)NCCN3CCC(C(=O)OC4CCN(C)CC4)CC3)c3cnc(Cl)cc31)OCCN2C(=O)OC(C)(C)C] | Int 11d and Int 2c | (Method 6): Rt = 0.60 min, ES⁺ m/z 712.3/714.4 [M + H]⁺ |
| Intermediate 13ai | 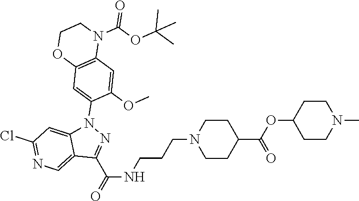 [SMILES: COc1cc2c(cc1-n1nc(C(=O)NCCCN3CCC(C(=O)OC4CCN(C)CC4)CC3)c3cnc(Cl)cc31)OCCN2C(=O)OC(C)(C)C] | Int 11e and Int 2c | (Method 6): Rt = 0.62 min, ES⁺ m/z 726.2/728.3 [M + H]⁺ |

### Intermediate 14a

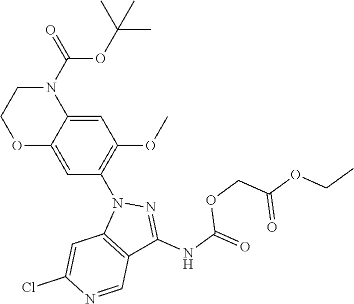 [SMILES: CCOC(=O)COC(=O)Nc1nn(-c2cc3c(cc2OC)N(C(=O)OC(C)(C)C)CCO3)c2cc(Cl)ncc12]

### tert-Butyl 7-(6-chloro-3-(((2-ethoxy-2-oxoethoxy)carbonyl)amino)-1H-pyrazolo[4,3-c]pyridin-1-yl)-6-methoxy-2,3-dihydro-4H-benzo[b][1,4]oxazine-4-carboxylate (Intermediate 14a)

A solution of intermediate 10 (90.0 mg, 0.21 mmol) in DCM (5 mL) was cooled to −78° C., then TEA (87.1 μL, 0.63 mmol) and bis(trichloromethyl)carbonate (61.8 mg, 0.21 mmol) were added. RM was stirred at −78° C. for 1 h. Ethyl 2-hydroxyacetate (79 μL, 0.83 mmol) and TEA (58.1 μL, 0.42 mmol) were added and RM stirred overnight reaching RT. RM was quenched with sat. aq. NaHCO₃ and water, then extracted with DCM (4×10 mL). Combined organic layers were washed with sat. aq. NaCl, dried over Na₂SO₄ and solvent evaporated in vacuo. The residue was purified by flash chromatography on Si cartridge by eluting with 0-40% EtOAc in DCM to afford the title product (92 mg).

LCMS (Method 1): Rt=1.29, ES⁺ m/z 562.1/564.1.

### Intermediates 14b to 14c

The following intermediates were prepared in a similar manner to intermediate 14a from the indicated starting materials.

| | | Starting | |
|---|---|---|---|
| Intermediate | Structure | materials | LCMS |
| Intermediate 14b | 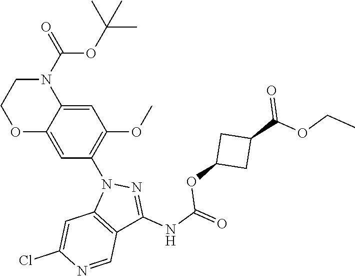 [SMILES: CCOC(=O)[C@H]1C[C@@H](OC(=O)Nc2nn(-c3cc4c(cc3OC)N(C(=O)OC(C)(C)C)CCO4)c3cc(Cl)ncc23)C1] | Int 10 and ethyl (1s,3s)- 3-hydroxy- cyclobutane- 1-carboxylate | (Method 1): Rt = 1.36 min, ES⁺ m/z 602.2/604.2 [M + H]⁺ |
| Intermediate 14c | 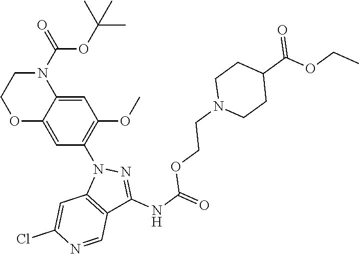 [SMILES: CCOC(=O)C1CCN(CCOC(=O)Nc2nn(-c3cc4c(cc3OC)N(C(=O)OC(C)(C)C)CCO4)c3cc(Cl)ncc23)CC1] | Int 10 and ethyl 1-(2- hydroxyethyl)- piperidine-4- carboxylate | (Method 1): Rt = 1.01 min, ES⁺ m/z 659.3/661.3 [M + H]⁺ |

### Intermediate 14d

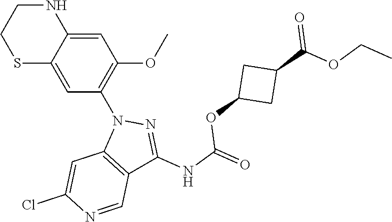 [SMILES: CCOC(=O)[C@H]1C[C@@H](OC(=O)Nc2nn(-c3cc4c(cc3OC)NCCS4)c3cc(Cl)ncc23)C1]

### Ethyl (1s,3s)-3-(((6-chloro-1-(6-methoxy-3,4-dihydro-2H-benzo[b][1,4]thiazin-7-yl)-1H-pyrazolo[4,3-c]pyridin-3-yl)carbamoyl)oxy)cyclobutane-1-carboxylate (Intermediate 14d)

To a solution of intermediate 6 (50.0 mg, 0.13 mmol) in 2-Me-THF (1.0 mL), T3P® (50.0% solution in EtOAc, 119 μL, 0.26 mmol), azido(trimethyl)silane (35.2 μL, 0.26 mmol) and TEA (55.5 μL, 0.40 mmol) were added. RM was refluxed for 1 h before addition of a solution of ethyl (1s,3s)-3-hydroxycyclobutane-1-carboxylate (38.3 mg, 0.26 mmol) and TEA (55.5 μL) in 2-Me-THF (150 μL). RM was stirred at RT and refluxed overnight. After cooling to RT, RM was diluted with EtOAc (25 mL), washed with sat. aq. NaHCO₃ (3×15 mL) and sat. aq. NaCl (15 mL). Organic layer was dried over Na₂SO₄ and concentrated in vacuo. The residue was purified by flash chromatography on Si cartridge by eluting with 0-50% EtOAc in cyclohexane to afford the title product (27 mg).

LCMS (Method 1): Rt=1.08, ES⁺ m/z 518.0/520.1.

### Intermediates 14e to 14f

The following intermediates were prepared in a similar manner to intermediate 14d from the indicated starting materials.

| | | Starting | |
|---|---|---|---|
| Intermediate | Structure | material | LCMS |
| Intermediate 14e | 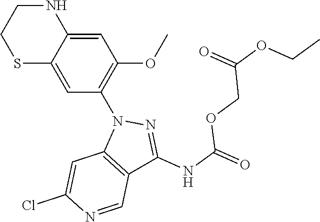 [SMILES: CCOC(=O)COC(=O)Nc1nn(-c2cc3c(cc2OC)NCCS3)c2cc(Cl)ncc12] | Int 6 and ethyl 2- hydroxy- acetate | (Method 1): Rt = 1.09 min, ES⁺ m/z 478.1/479.9 [M + H]⁺ |
| Intermediate 14f | 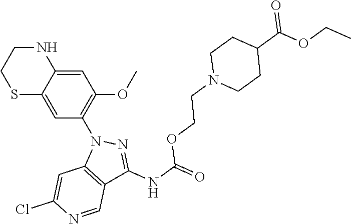 [SMILES: CCOC(=O)C1CCN(CCOC(=O)Nc2nn(-c3cc4c(cc3OC)NCCS4)c3cc(Cl)ncc23)CC1] | Int 6 and ethyl 1-(2- hydroxy- ethyl)piper- idine-4- carboxylate | (Method 1): Rt = 0.84 min, ES⁺ m/z 575.2/577.1 [M + H]⁺ |

### Intermediate 15

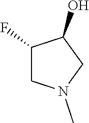

### (3S,4S)-4-fluoro-1-methylpyrrolidin-3-ol (Intermediate 15)

A mixture of (3S,4S)-4-fluoropyrrolidin-3-ol hydrochloride (50 mg, 0.35 mmol), iodomethane (22.0 μL, 0.35 mmol), and Na₂CO₃ (75 mg, 0.71 mmol) in MeCN (1.0 mL) were stirred at 80° C. for 18 h. RM was cooled to RT and filtered, collected solids washed with DCM (10 mL) and combined organics evaporated in vacuo. The residue was purified by flash chromatography on Si cartridge by eluting with 0-100% DCM:MeOH:NH₄OH (90:15:0.5) in DCM to afford the title product (8 mg).

¹H NMR (500 MHz, CDCl₃) δ/ppm: 4.85-5.06 (m, 1H), 4.38 (m, 1H), 3.12-3.30 (m, 1H), 2.97 (br dd, J=9.9, 4.4 Hz, 1H), 2.67-2.79 (m, 2H), 2.47 (s, 3H).

### Intermediate 16

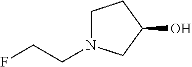

### (R)-1-(2-Fluoroethyl)pyrrolidin-3-ol (Intermediate 16)

A mixture of (3R)-pyrrolidin-3-ol (200 mg, 2.3 mmol), K₂CO₃ (577 mg, 4.2 mmol) and 1-bromo-2-fluoro-ethane (321 mg, 2.5 mmol) in MeCN (3.8 mL) was stirred at 70° C. overnight. After cooling to RT, RM was filtered through a plug of diacematous earth and washed with MeCN. Solvent was removed in vacuo to afford title product (282 mg) used in the next synthetic step without further purifications.

¹H-NMR (300 MHz, CDCl₃) δ: 4.54 (dt, J=47.6, 5.0 Hz, 2H), 4.31-4.37 (m, 1H), 2.91-2.98 (m, 1H), 2.78 (dt, J=28.0, 5.0 Hz, 2H), 2.71-2.76 (m, 1H), 2.60 (dd, J=10.2, 5.2 Hz, 1H), 2.37 (td, J=8.7, 6.5 Hz, 1H), 2.12-2.24 (m, 1H), 1.68-1.78 (m, 1H).

### Intermediate 17

### Step 1

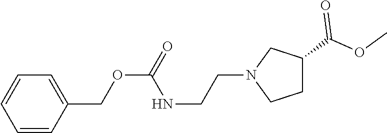 [SMILES: COC(=O)[C@@H]1CCN(CCNC(=O)OCc2ccccc2)C1]

### Methyl (R)-1-(2-(((benzyloxy)carbonyl)amino)ethyl)pyrrolidine-3-carboxylate (Intermediate 17-1)

A mixture of N-(2-bromoethyl)carbamate (250 mg, 0.97 mmol), methyl (R)-pyrrolidine-3-carboxylate hydrochloride (176 mg, 1.1 mmol) and K₂CO₃ (402 mg, 2.9 mmol) in MeCN (2 mL) was stirred at 70° C. overnight. After cooling to RT, RM was diluted with water and extracted with EtOAc (2×15 mL). Combined organics were washed with water (3×15 mL) and passed through phase separator. Solvent was removed affording product (282 mg) that was used in the next step without further purification.

¹H-NMR (500 MHz, CDCl₃) δ: 7.36-7.38 (m, 4H), 7.30-7.34 (m, 1H), 5.29 (bs, 1H), 5.11 (s, 2H), 3.69 (s, 3H), 3.29-3.35 (m, 2H), 2.98-3.04 (m, 1H), 2.85 (t, J=8.6 Hz, 1H), 2.70-2.73 (m, 1H), 2.53-2.68 (m, 4H), 2.06-2.10 (m, 2H).

### Step 2

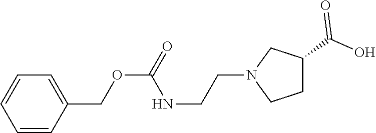 [SMILES: O=C(NCCN1CC[C@@H](C(=O)O)C1)OCc1ccccc1]

### (R)-1-(2-(((Benzyloxy)carbonyl)amino)ethyl)pyrrolidine-3-carboxylic acid (Intermediate 17-2)

Solution of Intermediate 17-1 (140 mg, 0.46 mmol) in ethanol (1.2 mL) was treated with NaOH (2.0 M in water, 560 μL, 1.1 mmol). RM was stirred at RT for 1h. RM was quenched with aq. 2N HCl (560 μL), then stirred at RT for 15 min. Ethanol was partially removed by evaporation, followed by acetone addition. RM was stirred for 15 min at RT and then filtered through a pad of diatomaceous earth, thoroughly washed with acetone. The filtrate was evaporated to dryness to give the title product (138 mg) that was used in the next synthetic steps without further purifications.

¹H-NMR (500 MHz, CDCl₃) δ: 7.54 (bs, 1H), 7.35-7.31 (m, 4H), 7.27-7.30 (m, 1H), 5.12 (m, 1H), 5.08 (s, 2H), 3.93 (m, 1H), 3.40-3.61 (m, 3H), 2.88-3.15 (m, 5H), 2.37-2.43 (m, 1H), 2.15-2.22 (m, 1H).

### Step 3

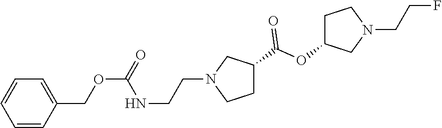 [SMILES: O=C(NCCN1CC[C@@H](C(=O)O[C@@H]2CCN(CCF)C2)C1)OCc1ccccc1]

### (R)-1-(2-Fluoroethyl)pyrrolidin-3-yl (R)-1-(2-(((benzyloxy)carbonyl)amino)ethyl)pyrrolidine-3-carboxylate (Intermediate 17)

To a solution of Intermediate 17-2 (135 mg, 0.46 mmol), Intermediate 16 (73.8 mg, 0.55 mmol) and 2,4,6-trichlorobenzoyl chloride (86.6 μL, 0.55 mmol) in dry THF (3 mL), TEA (129 μL, 0.92 mmol) and DMAP (14.1 mg, 0.25 mmol) were added and RM stirred at RT overnight. RM was poured in a mixture of EtOAc and water (10 mL each) and pH adjusted from 5.5 to 3.5 using aq. 1.0 M HCl. Aqueous layer was basified with 2M NaOH to pH 9.5 and extracted with EtOAc (2×10 mL). Combined organic layers were washed with sat. aq. NH₄Cl (2×), sat. aq. NaCl, dried over Na₂SO₄ and solvent removed in vacuo to afford the title product (162 mg).

¹H-NMR (500 MHz, CDCl₃) δ: 7.35-7.31 (m, 4H), 7.27-7.30 (m, 1H), 5.37 (bs, 1H), 5.18-5.22 (m, 1H), 5.11 (s, 2H), 4.56 (dt, J=47.4, 4.9 Hz, 2H), 3.32 (m, 2H), 2.71-3.03 (m, 8H), 2.57-2.67 (m, 4H), 2.48-2.52 (m, 1H), 2.24-2.31 (m, 1H), 2.05-2.09 (m, 2H), 2.81-2.88 (m, 1H).

### Intermediate 19

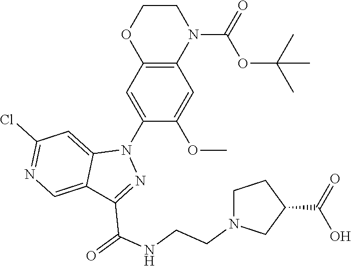 [SMILES: COc1cc2c(cc1-n1nc(C(=O)NCCN3CC[C@H](C(=O)O)C3)c3cnc(Cl)cc31)OCCN2C(=O)OC(C)(C)C]

### (S)-1-(2-(1-(4-(tert-Butoxycarbonyl)-6-methoxy-3,4-dihydro-2H-benzo[b][1,4]oxazin-7-yl)-6-chloro-1H-pyrazolo[4,3-c]pyridine-3-carboxamido)ethyl)pyrrolidine-3-carboxylic acid (Intermediate 19)

Aqueous solution of LiOH solution (1.0 M, 0.65 mL, 0.65 mmol) was added to a solution of Intermediate 13u (80.0 mg, 0.13 mmol) in THF (1.0 mL), an aqueous solution of LiOH (1.0 M, 0.65 mL, 0.65 mmol) was added and RM stirred at RT 30h. THF was evaporated in vacuo followed by addition of water, then pH was adjusted to 6.5. Aqueous mixture was extracted with DCM/i-PrOH (3×10 mL), combined organic layers passed through phase separator and solvent removed in vacuo to afford the title compound (68 mg).

LCMS (Method 1): Rt=0.94 min, ES⁺ m/z 601.3/603.2 [M+H]⁺

### Intermediate 20a

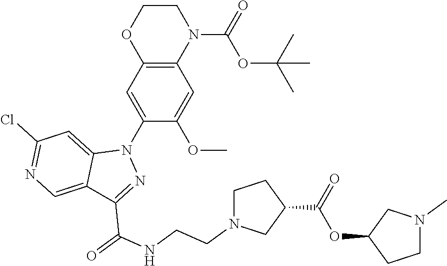 [SMILES: COc1cc2c(cc1-n1nc(C(=O)NCCN3CC[C@H](C(=O)O[C@@H]4CCN(C)C4)C3)c3cnc(Cl)cc31)OCCN2C(=O)OC(C)(C)C]

### tert-Butyl 7-(6-chloro-3-((2-((S)-3-((((R)-1-methylpyrrolidin-3-yl)oxy)carbonyl)pyridine-1-yl)ethyl)carbamoyl)-1H-pyrazolo[4,3-c]pyridine-1-yl)-6-methoxy-2,3-dihydro-4H-benzo[b][1,4]oxazine-4-carboxylate (Intermediate 20a)

To a solution of Intermediate 19 (40.0 mg, 66.6 μmol), 2,4,6-trichlorobenzoyl chloride (10.4 μL, 66.6 μmol) and (R)-1-methylpyrrolidin-3-ol (6.44 μL, 66.6 μmol) in dry THF (1 mL), TEA (18.6 μL, 0.13 mmol) and DMAP (2.03 mg, 16.6 μmol) were added and RM stirred at RT for 1 h. RM was diluted with EtOAc (15 mL) and washed with sat. aq. NaHCO₃ (3×10 mL) and sat. aq. NaCl (10 mL). Organics were dried over Na₂SO₄ and concentrated in vacuo. The residue was purified by flash chromatography on Si cartridge by eluting with 0-100% DCM/MeOH (9:1) in DCM to afford the desired product (42 mg).

LCMS (Method 2): Rt=1.27 min, ES⁺ m/z 684.3/686.3 [M+H]⁺

### Intermediate 20b

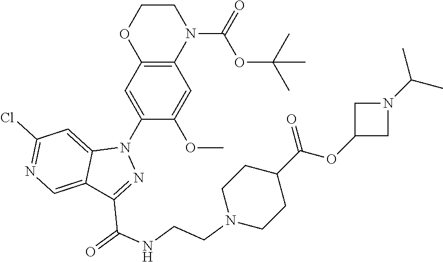 [SMILES: COc1cc2c(cc1-n1nc(C(=O)NCCN3CCC(C(=O)OC4CN(C(C)C)C4)CC3)c3cnc(Cl)cc31)OCCN2C(=O)OC(C)(C)C]

### tert-Butyl 7-(6-chloro-3-((2-(4-(((1-isopropylazetidin-3-yl)oxy)carbonyl)piperidin-1-yl)ethyl)carbamoyl)-1H-pyrazolo[4,3-c]pyridin-1-yl)-6-methoxy-2,3-dihydro-4H-benzo[b][1,4]oxazine-4-carboxylate (Intermediate 20b)

Title compound was prepared on a similar manner to Intermediate 20a, starting from Intermediate 12 (80 mg, 0.13 mmol) and 1 eq. of 1-isopropylazetidin-3-ol.

LCMS (Method 2): Rt=1.36 min, ES⁺ m/z 712.3/715.4 [M+H]⁺

### PREPARATION OF EXAMPLES

### Example 1

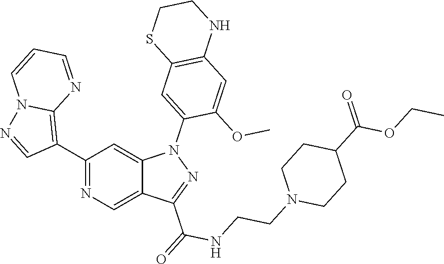 [SMILES: CCOC(=O)C1CCN(CCNC(=O)c2nn(-c3cc4c(cc3OC)NCCS4)c3cc(-c4cnn5cccnc45)ncc23)CC1]

### Ethyl 1-(2-(1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridine-3-carboxamido)ethyl)piperidine-4-carboxylate (Example 1)

A degassed mixture of intermediate 11c (80.0 mg, 0.14 mmol), 3-(4,4,5,5-tetramethyl-1,3,2-dioxaborolan-2-yl)pyrazolo[1,5-a]pyrimidine (49 mg, 0.200 mmol), XPhos Pd G3 (6 mg, 7 μmol), K₃PO₄ (0.5 M, 572 μL, 0.29 mmol) in a mixture THF/water (1/1, 4.57 mL) was heated at 65° C. for 3.5 h. A further equivalent of of 3-(4,4,5,5-tetramethyl-1,3,2-dioxaborolan-2-yl)pyrazolo[1,5-a]pyrimidine and XPhos Pd G3 were added and RM further heated at 100° C. for 1 h. After cooling to RT, RM was diluted with water and extracted with DCM (3×15 mL). Combined organic layers were passed through phase separator and solvent removed in vacuo. The residue was purified by flash chromatography on Si cartridge by eluting with 0-90% DCM/EtOH (90:1) in DCM to afford the desired product (62 mg).

LCMS (Method 3): Rt=4.42 min, ES⁺ m/z 642.3 [M+H]⁺

¹H-NMR (500 MHz, DMSO-d₆) δ: 9.45 (d, J=1.2 Hz, 1H), 9.23 (dd, J=7.1, 1.7 Hz, 1H), 8.90 (s, 1H), 8.73 (dd, J=4.0, 1.7 Hz, 1H), 8.46 (br t, J=5.8 Hz, 1H), 8.23 (d, J=1.0 Hz, 1H), 7.16 (dd, J=7.0, 4.2 Hz, 1H), 7.08 (s, 1H), 6.63-6.68 (m, 1H), 6.44 (s, 1H), 4.04 (q, J=7.1 Hz, 2H), 3.64 (s, 3H), 3.54-3.62 (m, 2H), 3.36-3.50 (m, 2H), 2.94-3.04 (m, 2H), 2.79-2.91 (m, 2H), 2.42-2.53 (m, 2H), 2.19-2.35 (m, 1H), 2.03 (br t, J=10.3 Hz, 2H), 1.69-1.86 (m, 2H), 1.45-1.65 (m, 2H), 1.16 (t, J=7.1 Hz, 3H).

### Examples 2 to 8

The following Examples were prepared in a similar manner to Example 1 from the indicated intermediate. It is additionally stated if base, solvent, temperature ligand and/or palladium source were varied.

| Example | Structure/name | Intermediate | ¹H-NMR | LCMS |
|---|---|---|---|---|
| Example 2 | 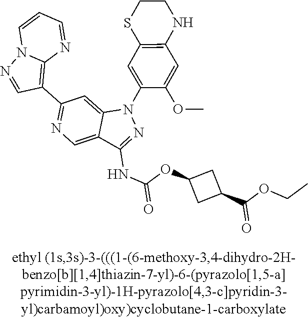 [SMILES: CCOC(=O)[C@H]1C[C@@H](OC(=O)Nc2nn(-c3cc4c(cc3OC)NCCS4)c3cc(-c4cnn5cccnc45)ncc23)C1] | Int 14d | (500 MHz, DMSO-d₆) δ: 10.48 (br s, 1H), 9.20-9.23 (m, 2H), 8.87 (s, 1H), 8.72 (dd, J = 4.0 Hz, J = 1.5 Hz, 1H), 8.08 (s, 1H), 7.14- 7.17 (m, 1H), 6.93 (s, 1H), 6.57 (br s, 1H), 6.42 (s, 1H), 5.01 (quin, J = 7.6 Hz, 1H), 4.09 (q, J = 7.1 Hz, 2H), 3.64 (s, 3H), 3.56-3.61 (m, 2H), 2.96-3.01 (m, 2H), 2.82-2.90 (m, 1H), 2.61- 2.68 (m, 2H), 2.22-2.30 (m, 2H), 1.20 (t, J = 7.3 Hz, 3H). | (Method 3): Rt = 5.30 min, ES⁺ m/z 601.2 [M + H]⁺ |
| Example 3 | 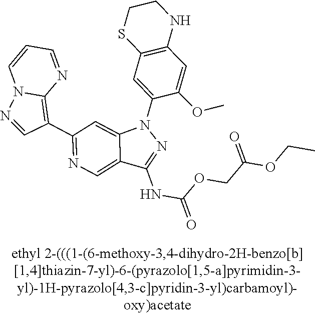 [SMILES: CCOC(=O)COC(=O)Nc1nn(-c2cc3c(cc2OC)NCCS3)c2cc(-c3cnn4cccnc34)ncc12] | Int 14e | (300 MHz, DMSO-d₆) δ: 10.75 (br s, 1H), 9.16-9.25 (m, 2H), 8.86 (s, 1H), 8.71 (dd, J = 4.1, 1.7 Hz, 1H), 8.08 (d, J = 1.0 Hz, 1H), 7.15 (dd, J = 7.1, 4.1 Hz, 1H), 6.95 (s, 1H), 6.54-6.62 (m, 1H), 6.42 (s, 1H), 4.77 (s, 2H), 4.20 (q, J = 7.0 Hz, 2H), 3.63 (s, 3H), 3.53-3.61 (m, 2H), 2.92-3.03 (m, 2H), 1.24 (t, J = 7.1 Hz, 3H) | (Method 3): Rt = 4.91 min, ES⁺ m/z 561.1 [M + H]⁺ |
| Example 4 | 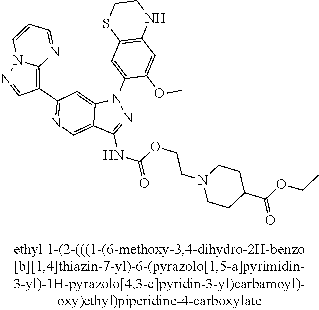 [SMILES: CCOC(=O)C1CCN(CCOC(=O)Nc2nn(-c3cc4c(cc3OC)NCCS4)c3cc(-c4cnn5cccnc45)ncc23)CC1] | Int 14f | (300 MHz, DMSO-d₆) δ: 10.42 (br s, 1H), 9.26 (d, J = 0.7 Hz, 1H), 9.21 (dd, J = 7.1, 1.7 Hz, 1H), 8.86 (s, 1H), 8.71 (dd, J = 4.1, 1.7 Hz, 1H), 8.07 (d, J = 0.9 Hz, 1H), 7.15 (dd, J = 7.1, 4.1 Hz, 1H), 6.92 (s, 1H), 6.53-6.61 (m, 1H), 6.41 s, 1H), 4.26 (br t, J = 5.6 Hz, 2H), 4.05 (q, J = 7.0 Hz, 2H), 3.63 (s, 3H), 3.54-3.61 (m, 2H), 2.93-3.02 (m, 2H), 2.82-2.93 (m, 2H), 2.60 (br t, J = 5.3 Hz, 2H), 2.20-2.34 (m, 1H), 1.98-2.15 (m, 2H), 1.72-1.87 (m, 2H), 1.51- 1.70 (m, 2H), 1.16 (t, J = 7.1 Hz, 3H). | (Method 3): Rt = 4.09 min, ES⁺ m/z 658.3 [M + H]⁺ |
| Example 5 | 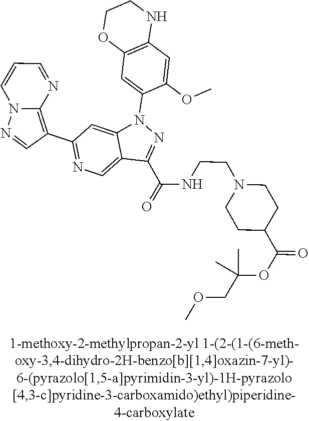 [SMILES: COCC(C)(C)OC(=O)C1CCN(CCNC(=O)c2nn(-c3cc4c(cc3OC)NCCO4)c3cc(-c4cnn5cccnc45)ncc23)CC1] | Int 13ac | (600 MHz, DMSO-d₆) δ: 9.47 (d, J = 1.3 Hz, 1H), 9.24 (dd, J = 7.0, 1.7 Hz, 1H), 8.92 (s, 1H), 8.74 (dd, J = 4.0, 1.7 Hz, 1H), 8.45 (t, J = 5.8 Hz, 1H), 8.25 (d, J = 1.1 Hz, 1H), 7.18 (dd, J = 7.0, 4.0 Hz, 1H), 6.89 (s, 1H), 6.50 (s, 1H), 6.39 (s, 1H), 4.15 (t, J = 4.4 Hz, 2H), 3.64 (s, 3H), 3.42-3.47 (m, 4H), 3.40 (br d, J = 2.2 Hz, 2H), 3.28 (s, 3H), 2.84 (br d, J = 10.1 Hz, 2H), 2.48 (m, 2H), 2.19 (br t, J = 11.1 Hz, 1H), 2.04 (br t, J = 10.9 Hz, 2H), 1.75 (br d, J = 10.3 Hz, 2H), 1.50-1.57 (m, 2H), 1.36 (s, 6H) | (Method 3): Rt = 4.41 min, ES⁺ m/z 684.3 [M + H]⁺ |
| Example 6 | 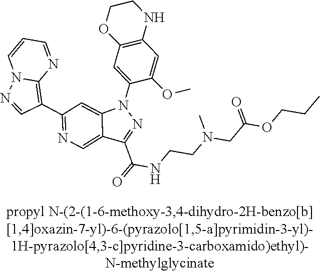 [SMILES: CCCOC(=O)CN(C)CCNC(=O)c1nn(-c2cc3c(cc2OC)NCCO3)c2cc(-c3cnn4cccnc34)ncc12] | Int 13ad | (600 MHz, DMSO-d₆) δ: 9.47 (d, J = 1.3 Hz, 1H), 9.24 (dd, J = 7.0, 1.7 Hz, 1H), 8.92 (s, 1H), 8.74 (dd, J = 4.0, 1.7 Hz, 1H), 8.40 (t, J = 5.5 Hz, 1H), 8.25 (d, J = 1.1 Hz, 1H), 7.18 (dd, J = 6.9, 4.1 Hz, 1H), 6.89 (s, 1H), 6.50 (s, 1H), 6.39 (s, 1H), 4.15 (t, J = 4.4 Hz, 2H), 3.99 (t, J = 6.7 Hz, 2H), 3.64 (s, 3H), 3.43 (q, J = 6.4 Hz, 2H), 3.40 (m, 2H), 3.37 (m, 2H), 2.73 (br t, J = 6.2 Hz, 2H), 2.37 (s, 3H), 1.57 (sxt, J = 7.2 Hz, 2H), 0.86 (t, J = 7.4 Hz, 3H) | (Method 3): Rt = 4.22 min, ES⁺ m/z 600.3 [M + H]⁺ |
| Example 7 | 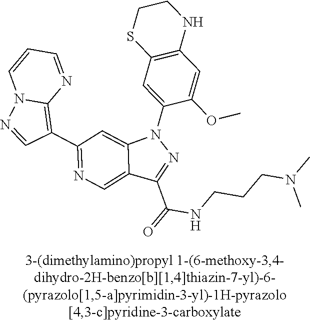 [SMILES: COc1cc2c(cc1-n1nc(C(=O)NCCCN(C)C)c3cnc(-c4cnn5cccnc45)cc31)SCCN2] | Int 13ae | (300 MHz, DMSO-d₆) δ: 9.38 (d, J = 1.2 Hz, 1H), 9.24 (dd, J = 7.1, 1.7 Hz, 1H), 8.90 (s, 1H), 8.74 (dd, J = 4.1, 1.7 Hz, 1H), 8.26 (d, J = 1.2 Hz, 1H), 7.17 (dd, J = 7.0, 4.2 Hz, 1H), 7.06 (s, 1H), 6.67-6.74 (m, 1H), 6.45 (s, 1H), 4.44 (t, J = 6.5 Hz, 2H), 3.64 (s, 3H), 3.56-3.62 (m, 2H), 2.94-3.04 (m, 2H), 2.38 (t, J = 7.1 Hz, 2H), 2.16 (s, 6H), 1.94 (quin, J = 6.8 Hz, 2H) | (Method 3): Rt = 4.30 min, ES⁺ m/z 545.1 [M + H]⁺ |
| Example 8 | 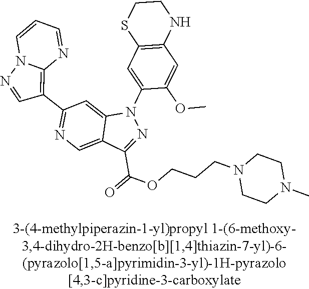 [SMILES: COc1cc2c(cc1-n1nc(C(=O)OCCCN3CCN(C)CC3)c3cnc(-c4cnn5cccnc45)cc31)SCCN2] | Int 13af | (300 MHz, DMSO-d₆) δ: 9.38 (d, J = 1.0 Hz, 1H), 9.24 (dd, J = 7.1, 1.7 Hz, 1H), 8.90 (s, 1H), 8.74 (dd, J = 4.1, 1.7 Hz, 1H), 8.25 (d, J = 1.2 Hz, 1H), 7.17 (dd, J = 7.0, 4.2 Hz, 1H), 7.06 (s, 1H), 6.66-6.76 (m, 1H), 6.45 (s, 1H), 4.44 (t, J = 6.5 Hz, 2H), 3.63 (s, 3H), 3.54-3.61 (m, 2H), 2.92-3.04 (m, 2H), 2.21- 2.46 (m, 10H), 2.11 (s, 3H), 1.95 (quin, J = 6.7 Hz, 2H). | (Method 4): Rt = 4.10 min, ES⁺ m/z 600.1 [M + H]⁺ |

### Example 9

### Step 1

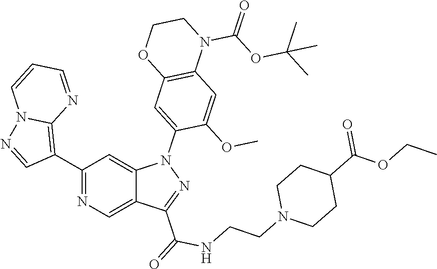 [SMILES: CCOC(=O)C1CCN(CCNC(=O)c2nn(-c3cc4c(cc3OC)N(C(=O)OC(C)(C)C)CCO4)c3cc(-c4cnn5cccnc45)ncc23)CC1]

### tert-Butyl 7-(3-((2-(4-(ethoxycarbonyl)piperidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 9-intermediate 1)

A degassed mixture of intermediate 11a (500 mg, 0.78 mmol), 3-(4,4,5,5-tetramethyl-1,3,2-dioxaborolan-2-yl)pyrazolo[1,5-a]pyrimidine (286 mg, 1.17 mmol), K₃PO₄ (330 mg, 1.55 mmol) and XPhos PdG3 (66 mg, 78 μmol) in water (4.5 mL)/THF (9 mL) was stirred under nitrogen at 65° C. for 1 h. After cooling to RT, RM was diluted with water (40 mL) and extracted with EtOAc (40 mL and 2×15 mL). Combined organic layers were washed with water, sat. aq. NaCl (40 mL each), dried over Na₂SO₄ and evaporated in vacuo. The residue was purified by flash chromatography on Si cartridge eluting with 0-65% DCM/MeOH/NH₄OH (90:5:0.5) in DCM to afford the product (483 mg).

LCMS (Method 1), Rt=0.91, ES⁺ m/z 726.4.

### Step 2

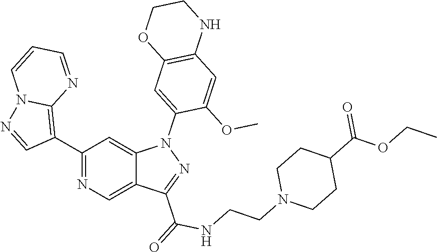 [SMILES: CCOC(=O)C1CCN(CCNC(=O)c2nn(-c3cc4c(cc3OC)NCCO4)c3cc(-c4cnn5cccnc45)ncc23)CC1]

### Ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate (Example 9)

To a solution of example 9-intermediate 1 (480 mg, 0.66 mmol) in DCM (10 mL), TFA (2.06 mL, 27.8 mmol) was added, and RM stirred at RT overnight. RM was evaporated in vacuo and the residue loaded on a SCX cartridge, washed with ethanol, and eluted with 15% aqueous ammonia in ethanol. The crude material thus obtained was purified by flash chromatography on Si cartridge by eluting with 0-100% DCM/MeOH/NH₄OH (90:9:0.5) in DCM to afford the title product (246 mg).

LCMS (Method 3), Rt=4.13 min, ES⁺ m/z 626.3 [M+H]⁺

¹H-NMR (500 MHz, DMSO-d₆) δ: 9.46 (s, 1H), 9.23 (br d, J=6.7 Hz, 1H), 8.91 (s, 1H), 8.76-8.72 (m, 1H), 8.45 (br s, 1H), 8.24 (s, 1H), 7.20-7.15 (m, 1H), 6.89 (s, 1H), 6.49 (s, 1H), 6.38 (br s, 1H), 4.14 (br s, 2H), 4.05 (q, J=6.8 Hz, 2H), 3.63 (s, 3H), 3.48-3.36 (m, 5H), 2.86 (br d, J=10.7 Hz, 2H), 2.47-2.44 (overlapped with DMSO, 1H), 2.28 (br s, 1H), 2.04 (br t, J=10.7 Hz, 2H), 1.79 (br d, J=11.6 Hz, 2H), 1.56 (br d, J=9.8 Hz, 2H), 1.17 (t, J=7.2 Hz, 3H)

### Examples 10 to 42

The following Examples were prepared with a two step process similarly to Example 9 from the indicated starting materials. It is additionally stated if base, solvent, temperature ligand and/or palladium source were varied.

| | | Step 1 | | | |
|---|---|---|---|---|---|
| | | Starting | Name of intermediate | Step 2 | |
| Example | Structure/name | material | Step 1/LCMS | ¹H-NMR | LCMS |
| Example 10 | 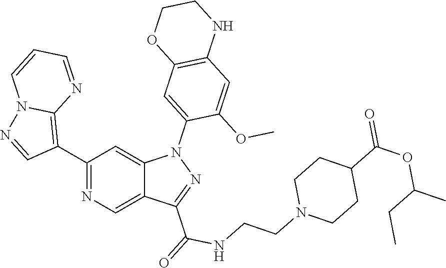 [SMILES: CCC(C)OC(=O)C1CCN(CCNC(=O)c2nn(-c3cc4c(cc3OC)NCCO4)c3cc(-c4cnn5cccnc45)ncc23)CC1] | Int 13c | tert-butyl 7-(3-((2-(4- (sec- butoxycarbonyl)piperidin- 1-yl)ethyl)carbamoyl)- 6-(pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 2): Rt = 1.44 min, ES⁺ m/z 754.5 [[M + H]⁺ | (600 MHz, DMSO-d₆) δ: 9.46 (d, J = 1.2 Hz, 1H), 9.23 (dd, J = 7.1, 1.6 Hz, 1H), 8.91 (s, 1H), 8.73 (dd, J = 4.1, 1.6 Hz, 1H), 8.43 (t, J = 5.7 Hz, 1H), 8.24 (d, J = 1.4 Hz, 1H), 7.16 (dd, J = 7.1 Hz, 3.9 Hz, 1H), 6.88 (s, 1H), 6.49 (s, 1H), 6.38 (t, J = 2.8 Hz, 1H), 4.70-4.78 (m, 1H), 4.13 (t, J = 4.2 Hz, 2H), 3.63 (s, 3H), 3.42 (q, J = 6.4 Hz, 2H), 3.37-3.41 (m, 2H), 2.83-2.88 (m, 2H), 2.46-2.55 (overlapped with DMSO, 2H), 2.24- 2.30 (m, 1H), 2.02-2.08 (m, 2H), 1.75- 1.82 (m, 2H), 1.47-1.54 (m, 2H), 1.54- 1.60 (m, 2H), 1.13 (d, J = 6.8 Hz, 3H), 0.82 (t, J = 7.0 Hz, 3H) | (Method 3): Rt = 4.65 min, ES⁺ m/z 654.3 [M + H]⁺ |
| | sec-butyl 1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 11 | 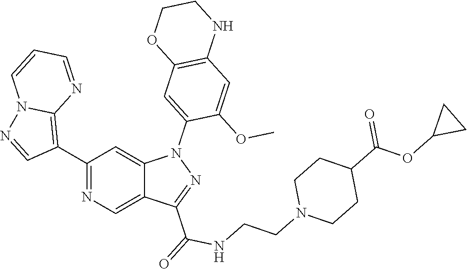 [SMILES: COc1cc2c(cc1-n1nc(C(=O)NCCN3CCC(C(=O)OC4CC4)CC3)c3cnc(-c4cnn5cccnc45)cc31)OCCN2] | Int 13b | tert-butyl 7-(3-((2-(4- (cyclopropoxycarbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.95 min, ES⁺ m/z 738.4 [M + H]⁺ | (500 MHz, DMSO-d₆) δ: 9.45 (d, J = 0.7 Hz, 1H), 9.23 (dd, J = 7.1 Hz, 1H), 8.91 (s, 1H), 8.73 (dd, J = 3.9, 1.6 Hz, 1H), 8.44 (s, 1H), 8.24 (d, J = 0.7 Hz, 1H), 7.17 (dd, J = 6.9, 3.9 Hz, 1H), 6.88 (s, 1H), 6.49 (s, 1H), 6.37-6.39 (m, 1H), 4.12-4.15 (m, 2H), 4.02-4.07 (m, 1H), 3.63 (s, 3H), 3.36-3.42 (m, 2H), 3.26- 3.34 (m, 2H, overlapped with HDO); 2.80-2.90 (m, 2H), 2.45-2.57 (m, 2H, overlapped with DMSO); 2.21-2.29 (m, 1H), 1.98-2.07 (m, 2H), 1.73-1.82 (m, 2H), 1.48-1.60 (m, 2H), 0.62-0-70 (m, 2H), 0.57-0.62 (m, 2H) | (Method 3): Rt = 4.22 min, ES⁺ m/z 638.3 [M + H]⁺ |
| | cyclopropyl 1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 12 | 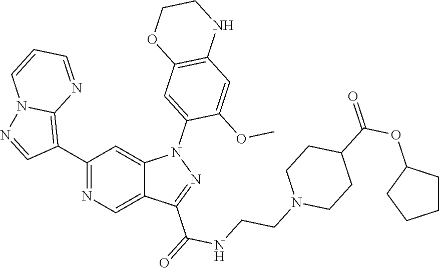 [SMILES: COc1cc2c(cc1-n1nc(C(=O)NCCN3CCC(C(=O)OC4CCCC4)CC3)c3cnc(-c4cnn5cccnc45)cc31)OCCN2] | Int13a | tert-butyl 7-(3-((2-(4- ((cyclopentyloxy)carbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 2): Rt = 1.44 min, ES⁺ m/z 766.5 [M + H]⁺ | (600 MHz, DMSO-d₆) δ: 9.45 (d, J = 1.4 Hz, 1H), 9.24 (dd, J = 6.8, 1.6 Hz, 1H), 8.91 (s, 1H), 8.73 (dd, J = 4.1, 1.9 Hz, 1H), 8.43 (t, J = 6.0 Hz, 1H), 8.24 (d, J = 1.0 Hz, 1H), 7.16 (dd, J = 7.0 Hz, 4.0 Hz, 1H), 6.88 (s, 1H), 6.49 (s, 1H), 6.37-6.39 (m, 1H), 5.04-5.07 (m, 1H), 4.13 (t, J = 4.4 Hz, 2H), 3.63 (s, 3H), 3.42 (q, J = 6.4 Hz, 2H), 3.37-3.41 (m, 2H), 2.80-2.88 (m, 2H), 2.46-2.52 (overlapped with DMSO, 2H), 2.20- 2.28 (m, 1H), 2.00-2.08 (m, 2H), 1.73- 1.84 (m, 4H), 1.60-1.69 (m, 2H), 1.49- 1.59 (m, 6H) | (Method 3): Rt = 4.69 min, ES⁺ m/z 666.3 [M + H]⁺ |
| | cyclopentyl 1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 13 | 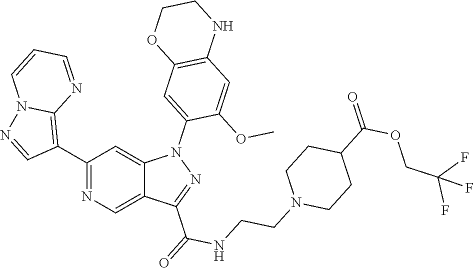 [SMILES: COc1cc2c(cc1-n1nc(C(=O)NCCN3CCC(C(=O)OCC(F)(F)F)CC3)c3cnc(-c4cnn5cccnc45)cc31)OCCN2] | Int 13d | tert-butyl 6-methoxy-7- (6-(pyrazolo[1,5- a]pyrimidin-3-yl)-3-((2- (4-((2,2,2- trifluoroethoxy)carbonyl) piperidin-1- yl)ethyl)carbamoyl)-1H- pyrazolo[4,3-c]pyridin-1- yl)-2,3-dihydro-4H- benzo[b][1,4]oxazine-4- carboxylate (Method 2): Rt = 0.98 min, ES⁺ m/z 780.5 [M + H]⁺ | (500 MHz, DMSO-d₆) δ: 9.46 (d, J = 1.0 Hz, 1H), 9.22 (dd, J = 7.0, 1.7 Hz, 1H), 8.91 (s, 1H), 8.73 (dd, J = 4.2, 1.6 Hz, 1H), 8.45 (t, J = 5.6 Hz, 1H), 8.24 (d, J = 0.9 Hz, 1H), 7.16 (dd, J = 7.1, 4.0 Hz, 1H), 6.88 (s, 1H), 6.49 (s, 1H), 6.38- 6.40 (m, 1H), 4.72 (q, J = 8.1 Hz, 2H), 4.13 (t, J = 4.0 Hz, 2H), 3.63 (s, 3H), 3.42 (q, J = 6.6 Hz, 2H), 3.31-3.41 (m, overlapped with HDO, 2H), 2.82-2.89 (m, 2H), 2.43-2.55 (m, overlapped with DMSO, 2H), 2.05-2.12 (m, 2H), 1.80- 1.87 (m, 2H), 1.53-1.64 (m, 3H) | (Method 3): Rt = 4.48 min, ES⁺ m/z 680.3 [M + H]⁺ |
| | 2,2,2-trifluoroethyl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 14 | 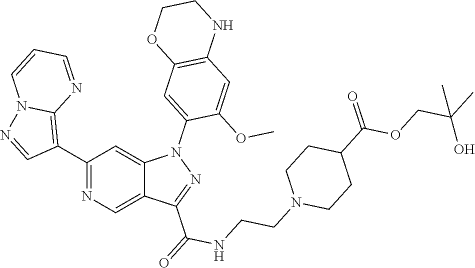 [SMILES: COc1cc2c(cc1-n1nc(C(=O)NCCN3CCC(C(=O)OCC(C)(C)O)CC3)c3cnc(-c4cnn5cccnc45)cc31)OCCN2] | Int 13e | tert-butyl 7-(3-((2-(4-((2- hydroxy-2- methylpropoxy)carbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.85 min, ES⁺ m/z 770.4 [M + H]⁺ | (600 MHz, DMSO-d₆) δ: 9.46 (d, J = 1.1 Hz, 1H), 9.24 (dd, J = 7.0, 1.7 Hz, 1H), 8.91 (s, 1H), 8.74 (dd, J = 4.1, 1.7 Hz, 1H), 8.45 (t, J = 5.9 Hz, 1H), 8.25 (d, J = 1.3 Hz, 1H), 7.17 (dd, J = 7.0, 4.3 Hz, 1H), 6.89 (s, 1H), 6.49 (s, 1H), 6.38 (br s, 1H), 4.58 (s, 1H), 4.14 (t, J = 4.0 Hz, 2H), 3.80 (s, 2H), 3.64 (s, 3H), 3.44 (q, J = 6.5 Hz, 2H), 3.38-3.41 (m, 2H), 2.84- 2.89 (br d, J = 11.4 Hz, 2H), 2.51 (m, overlapped with DMSO, 2H), 2.31-2.37 (m, 1H), 2.03-2.09 (m, 2H), 1.80-1.85 (m, 2H), 1.55-1.63 (m, 2H), 1.09 (s, 6H) | (Method 3): Rt = 3.94 min, ES⁺ m/z 670.1 [M + H]⁺ |
| | 2-hydroxy-2-methylpropyl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 15 | 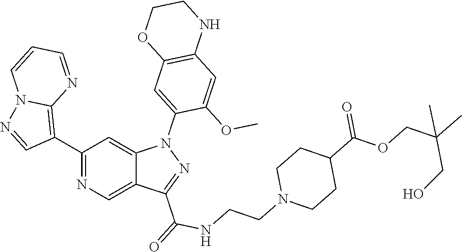 [SMILES: COc1cc2c(cc1-n1nc(C(=O)NCCN3CCC(C(=O)OCC(C)(C)CO)CC3)c3cnc(-c4cnn5cccnc45)cc31)OCCN2] | Int 13f | tert-butyl 7-(3-((2-(4-((3- hydroxy-2,2- dimethylpropoxy)carbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.93 min, ES⁺ m/z 784.6 [M + H]⁺ | (500 MHz, DMSO-d₆) δ: 9.46 (s, 1H), 9.24 (dd, J = 7.1, 1.4 Hz, 1H), 8.91 (s, 1H), 8.74 (dd, J = 4.1, 1.5 Hz, 1H), 8.47 (bs, 1H), 8.25 (s, 1H), 7.17 (dd, J = 7.0, 4.0 Hz, 1H), 6.88 (s, 1H), 6.49 (s, 1H), 6.39 (bs, 1H), 4.61 (t, J = 5.2 Hz, 1H), 4.14 (t, J = 4.1 Hz, 2H), 3.80 (s, 2H), 3.64 (s, 3H), 3.50-3.40 (m, 2H), 3.40- 3.36 (m, 2H), 3.17 (d, J = 5.4 Hz, 2H), 2.93-2.79 (m, 2H), 2.54 (m, 2H) overlapped with DMSO, 2.38-2.26 (m, 1H), 2.14-1.99 (m, 2H), 1.88-1.77 (m, 2H), 1.69-1.52 (m, 2H), 0.82 (s, 6H) | (Method 3): Rt = 4.15 min, ES⁺ m/z 684.3 [M + H]⁺ |
| | 3-hydroxy-2,2-dimethylpropyl 1-(2-(1- | | | | |
| | (6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 16 | 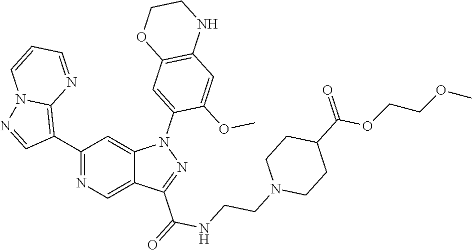 [SMILES: COCCOC(=O)C1CCN(CCNC(=O)c2nn(-c3cc4c(cc3OC)NCCO4)c3cc(-c4cnn5cccnc45)ncc23)CC1] | Int 13g | tert-butyl 6-methoxy-7- (3-((2-(4-((2- methoxyethoxy)carbonyl) piperidin-1- yl)ethyl)carbamoyl)-6- (pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 2): Rt = 1.22 min, ES⁺ m/z 756.4 [M + H]⁺ | (600 MHz, DMSO-d₆) δ: 9.46 (d, 1.7 Hz, 1H), 9.23 (dd, J = 7.1, 1.8 Hz, 1H), 8.92 (s, 1H), 8.74 (dd, J = 4.1, 1.7 Hz, 1H), 8.46 (t, J = 5,8 Hz, 1H), 8.25 (d, J = 1.2 Hz, 1H), 7.17 (dd, J = 7.1, 4.1 Hz, 1H), 6.89 (s, 1H), 6.50 (s, 1H), 6.38 (bs, 1H), 4.17-4.11 (m, 4H), 3.64 (s, 3H), 3.53-3.50 (m, 2H), 3.46-3.42 (m, 2H), 3.41-3.38 (m, 2H, overlapped with HDO), 3.25 (s, 3H), 2.89-2.83 (m, 2H), 2.53-2.51 (m, 2H, overlapped with DMSO), 2.35-2.28 (m, 1H), 2.09-2.02 (m, 2H), 1.83-1.77 (m, 2H), 1.61-1.52 (m, 2H) | (Method 3): Rt = 3.97 min, ES⁺ m/z 656.3 [M + H]⁺ |
| | 2-methoxyethyl 1-(2-(1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridine-3- | | | | |
| | carboxamido)ethyl)piperidine-4- | | | | |
| | carboxylate | | | | |
| Example 17 | 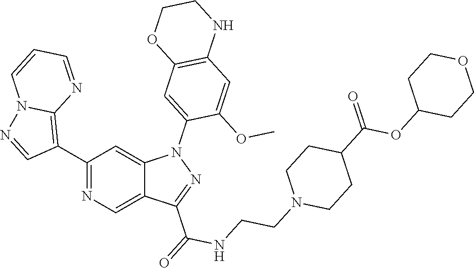 [SMILES: COc1cc2c(cc1-n1nc(C(=O)NCCN3CCC(C(=O)OC4CCOCC4)CC3)c3cnc(-c4cnn5cccnc45)cc31)OCCN2] | Int 13h | tert-butyl 6-methoxy-7- (6-(pyrazolo[1,5- a]pyrimidin-3-yl)-3-((2- (4-(((tetrahydro-2H- pyran-4- yl)oxy)carbonyl)piperidin- 1-yl)ethyl)carbamoyl)- 1H-pyrazolo[4,3- c]pyridin-1-yl)-2,3- dihydro-4H- benzo[b][1,4]oxazine-4- carboxylate (Method 1): Rt = 0.90 min, ES⁺ m/z 782.4 [M + H]⁺ | (500 MHz, DMSO-d₆) δ: 9.46 (d, J = 1.3 Hz, 1H), 9.24 (dd, J = 7.0, 1.8 Hz, 1H), 8.91 (s, 1H), 8.74 (dd, J = 4.0, 1.6 Hz, 1H), 8.45 (t, J = 5.9 Hz, 1H), 8.25 (d, J = 1.3 Hz, 1H), 7.15-7.19 (m, 1H), 6.89 (s, 1H), 6.49 (s, 1H), 6.38 (s, 1H), 4.86 (m, 1H), 4.11-4.17 (m, 2H), 3.72-3.79 (m, 2H), 3.64 (s, 3H), 3.41-3.48 (m, 4H), 3.37-3.41 (m, 2H), 2.86 (br d, J = 11.2 Hz, 2H), 2.46-2.52 (m, 2 H; overlapped with DMSO); 2.30 (tt, J = 11.0, 4.0 Hz, 1H), 2.02-2.10 (m, 2H): 1.76-1.86 (m, 4H), 1.47-1.62 (m, 4H) | (Method 3): Rt = 4.87 min, ES⁺ m/z 682.3 [M + H]⁺ |
| | tetrahydro-2H-pyran-4-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 18 | 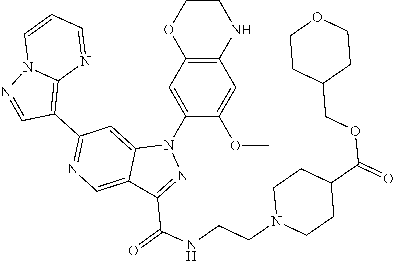 [SMILES: COc1cc2c(cc1-n1nc(C(=O)NCCN3CCC(C(=O)OCC4CCOCC4)CC3)c3cnc(-c4cnn5cccnc45)cc31)OCCN2] | Int 13ag | tert-butyl 6-methoxy-7- (6-(pyrazolo[1,5- a]pyrimidin-3-yl)-3-((2- (4-(((tetrahydro-2H- pyran-4- yl)methoxy)carbonyl) piperidin-1- yl)ethyl)carbamoyl)-1H- pyrazolo[4,3-c]pyridin-1- yl)-2,3-dihydro-4H- benzo[b][1,4]oxazine-4- carboxylate (Method 6): Rt = 0.78 min, ES⁺ m/z 796.5 [M + H]⁺ | (DMSO-d6, 400 MHz) δ 9.42 (s, 1H), 9.20 (dd, 1H, J = 1.3, 7.0 Hz), 8.88 (s, 1H), 8.70 (dd, 1H, J = 1.4, 4.1 Hz), 8.41 (t, 1H, J = 5.8 Hz), 8.21 (s, 1H), 7.14 (dd, 1H, J = 4.2, 7.0 Hz), 6.85 (s, 1H), 6.46 (s, 1H), 6.35 (s, 1H), 4.1-4.1 (m, 2H), 3.8- 3.9 (m, 4H), 3.60 (s, 3H), 3.3-3.4 (m, 4H), 3.2-3.3 (m, 3H), 2.83 (br d, 2H, J = 11.4 Hz), 2.2-2.3 (m, 1H), 2.02 (br t, 2H, J = 10.7 Hz), 1.7-1.9 (m, 3H), 1.5- 1.6 (m, 4H), 1.1-1.3 (m, 3H) | (Method 3): Rt = 4.37 min, ES⁺ m/z 696.2 [M + H]⁺ |
| | (tetrahydro-2H-pyran-4-yl)methyl 1-(2- | | | | |
| | (1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 19 | 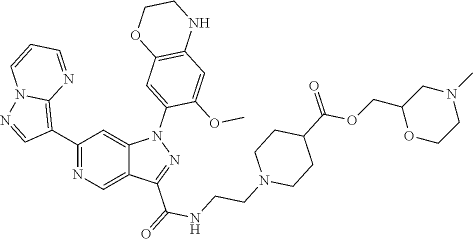 [SMILES: COc1cc2c(cc1-n1nc(C(=O)NCCN3CCC(C(=O)OCC4CN(C)CCO4)CC3)c3cnc(-c4cnn5cccnc45)cc31)OCCN2] | Int 13i | tert-butyl 6-methoxy-7- (3-((2-(4-(((4- methylmorpholin-2- yl)methoxy)carbonyl) piperidin-1- yl)ethyl)carbamoyl)-6- (pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.76 min, ES⁺ m/z 811.4 [M + H]⁺ | (600 MHz, DMSO-d₆) δ: 9.46 (d, J = 1.3 Hz, 1H), 9.24 (dd, J = 7.0, 1.6 Hz, 1H), 8.91 (s, 1H), 8.74 (dd, J = 4.1, 1.7 Hz, 1H), 8.45 (t, J = 5.9 Hz, 1H), 8.24 (d, J = 1.3 Hz, 1H), 7.15-7.20 (m, 1H), 6.89 (s, 1H), 6.49 (s, 1H), 6.38 (s, 1H), 4.12-4.17 (m, 2H), 4.01-4.07 (m, 1H), 3.95-4.00 (m, 1H), 3.73-3.79 (m, 1H), 3.64 (s, 3H), 3.59-3.63 (m, 1H), 3.41-3.50 (m, 3H), 3.37-3.41 (m, 2H), 2.82-2.91 (m, 2H), 2.63-2.66 (m, 1H), 2.53-2.57 (m, 1H), 2.46-2.52 (m, 2 H; overlapped with DMSO); 2.28-2.36 (m, 1H), 2.16 (s, 3H), 2.01-2.08 (m, 2H), 1.94 (td, J = 11.4, 3.30 Hz, 1H), 1.77-1.82 (m, 2H), 1.73-1.78 (m, 1H), 1.50-1.62 (m, 2H) | (Method 3): Rt = 3.42 min, ES⁺ m/z 711.3 [M + H]⁺ |
| | (4-methylmorpholin-2-yl)methyl 1-(2- | | | | |
| | (1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 20 | 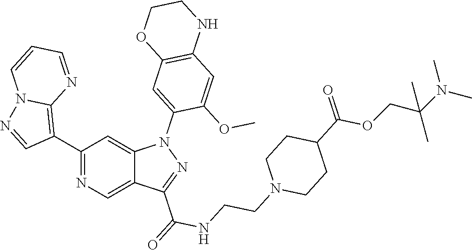 [SMILES: COc1cc2c(cc1-n1nc(C(=O)NCCN3CCC(C(=O)OCC(C)(C)N(C)C)CC3)c3cnc(-c4cnn5cccnc45)cc31)OCCN2] | Int 13j | tert-butyl 7-(3-((2-(4-((2- (dimethylamino)-2- methylpropoxy)carbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.77 min, ES⁺ m/z 797.6 [M + H]⁺ | (600 MHz, DMSO-d₆) δ: 9.46 (s, 1H), 9.23-9.26 (m, 1H), 8.91 (s, 1H), 8.73- 8.75 (m, 1H), 8.44-8.49 (m, 1H), 8.24 (s, 1H), 7.17 (dd, J = 7.0, 2.8 Hz, 1H), 6.88 (s, 1H), 6.49 (s, 1H), 6.38-6.40 (m, 1H), 4.14 (t, J = 4.3 Hz, 2H), 3.93 (s, 2H), 3.63 (s, 3H), 3.44 (q, J = 6.3 Hz, 2H), 3.38-3.41 (m, 2H), 2.83-2.90 (m, 2H), 2.49-2.53 (m, 2H, overlapped with DMSO); 2.28-2.35 (m, 1H), 2.17 (s, 6H), 2.02-2.08 (m, 2H), 1.80-1.86 (m, 2H), 1.55-1.63 (m, 2H), 0.98 (s, 6H) | (Method 3): Rt = 3.47 min, ES⁺ m/z 697.4 [M + H]⁺ |
| | 2-(dimethylamino)-2-methylpropyl 1- | | | | |
| | (2-(1-(6-methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 21 | 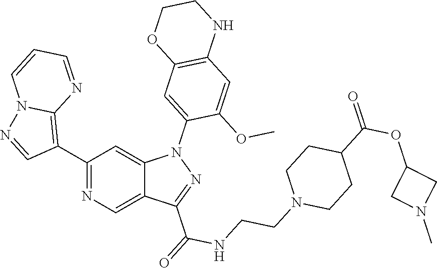 [SMILES: COc1cc2c(cc1-n1nc(C(=O)NCCN3CCC(C(=O)OC4CN(C)C4)CC3)c3cnc(-c4cnn5cccnc45)cc31)OCCN2] | Int 13k | tert-butyl 6-methoxy-7- (3-((2-(4-(((1- methylazetidin-3- yl)oxy)carbonyl)piperidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.74 min, ES⁺ m/z 767.5 [M + H]⁺ | (500 MHz, DMSO-d₆) δ: 9.45 (s, 1H), 9.23 (d, J = 7.0 Hz, 1H), 8.91 (d, J = 1.2 Hz, 1H), 8.73 (m, 1H), 8.44 (t, J = 6.1 Hz, 1H), 8.24 (s, 1H), 7.17 (dd, J = 5.8, 4.3 Hz, 1H), 6.88 (s, 1H), 6.49 (s, 1H), 6.38 (s, 1H), 4.87 (quin, J = 5.7 Hz, 1H), 4.14 (m, 2H), 3.63 (s, 3H), 3.54 (br t, J = 6.6 Hz, 2H), 3.43 (m, 2H), 3.37-3.40 (m, 2H), 2.87-2.91 (m, 2H), 2.84 (m, 2H), 2.50 (m, 2H), 2.28-2.35 (m, 1H), 2.22 (s, 3H), 2.05 (br t, J = 11.1 Hz, 2H), 1.80 (br d, J = 11.0 Hz, 2H), 1.55 (br d, J = 11.3 Hz, 2H) | (Method 3): Rt = 3.37 min, ES⁺ m/z 667.3 [M + H]⁺ |
| | 1-methylazetidin-3-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 22 | 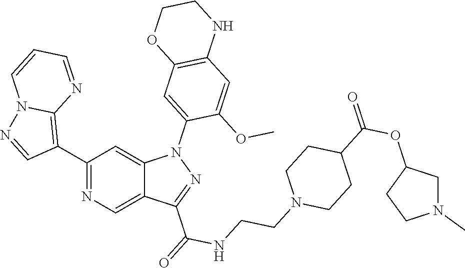 [SMILES: COc1cc2c(cc1-n1nc(C(=O)NCCN3CCC(C(=O)OC4CCN(C)C4)CC3)c3cnc(-c4cnn5cccnc45)cc31)OCCN2] | Int 13l | tert-butyl 6-methoxy-7- (3-((2-(4-(((1- methylpyrrolidin-3- yl)oxy)carbonyl)piperidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.74 min, ES⁺ m/z 781.6 [M + H]⁺ | (600 MHz, DMSO-d₆) δ: 9.46 (d, J = 1.2 Hz, 1H), 9.23 (dd, J = 7.0, 1.5 Hz, 1H), 8.91 (s, 1H), 8.73 (dd, J = 4.0, 1.8 Hz, 1H), 8.44 (t, J = 5.8 Hz, 1H), 8.24 (d, J = 1.2 Hz, 1H), 7.17 (m, 1H), 6.88 (s, 1H), 6.49 (s, 1H), 6.38 (s, 1H), 5.04 (m, 1H), 4.14 (t, J = 4.3 Hz, 2H), 3.63 (s, 3H), 3.43 (q, J = 6.4 Hz, 2H), 3.39 (m, 2H), 2.85 (m, 2H), 2.61 (m, 2H), 2.52 (m, 1H, overlapped with DMSO), 2.47 (m, 2H, overlapped with DMSO), 2.26 (m, 2H), 2.22 (s, 3H), 2.15 (m, 1H), 2.04 (t, J = 9.9 Hz, 2H), 1.78 (m, 2H), 1.65 (m, 1H), 1.55 (m, 2H) | (Method 3): Rt = 3.38 min, ES⁺ m/z 681.3 [M + H]⁺ |
| | 1-methylpyrrolidin-3-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 23 | 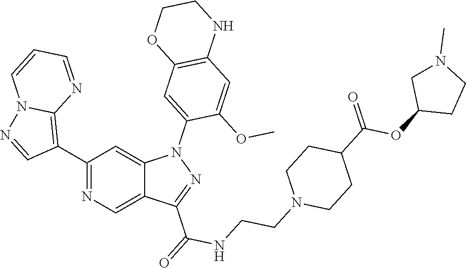 [SMILES: COc1cc2c(cc1-n1nc(C(=O)NCCN3CCC(C(=O)O[C@@H]4CCN(C)C4)CC3)c3cnc(-c4cnn5cccnc45)cc31)OCCN2] | Int 13m | tert-butyl (R)-6-methoxy- 7-(3-((2-(4-(((1- methylpyrrolidin-3- yl)oxy)carbonyl)piperidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.79 min, ES⁺ m/z 781.6 [M + H]⁺ | (500 MHz, DMSO-d₆) δ: 9.46 (d, J = 1.2 Hz, 1H), 9.23 (dd, J = 7.0, 1.5 Hz, 1H), 8.91 (s, 1H), 8.73 (dd, J = 4.0, 1.8 Hz, 1H), 8.44 (t, J = 5.8 Hz, 1H), 8.24 (d, J = 1.2 Hz, 1H), 7.17 (m, 1H), 6.88 (s, 1H), 6.49 (s, 1H), 6.38 (s, 1H), 5.04 (m, 1H), 4.14 (t, J = 4.3 Hz, 2H), 3.63 (s, 3H), 3.43 (q, J = 6.4 Hz, 2H), 3.39 (m, 2H), 2.85 (m, 2H), 2. 61 (m, 2H), 2.52 (m, 1H, overlapped with DMSO), 2.47 (m, 2H, overlapped with DMSO), 2.26 (m, 2H), 2.22 (s, 3H), 2.15 (m, 1H), 2.04 (t, J = 9.9 Hz, 2H), 1.78 (m, 2H), 1.65 (m, 1H), 1.55 (m, 2H) | (Method 3): Rt = 3.43 min, ES⁺ m/z 681.3 [M + H]⁺ |
| | (R)-1-methylpyrrolidin-3-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 24 | 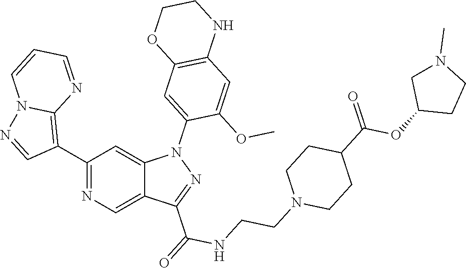 [SMILES: COc1cc2c(cc1-n1nc(C(=O)NCCN3CCC(C(=O)O[C@H]4CCN(C)C4)CC3)c3cnc(-c4cnn5cccnc45)cc31)OCCN2] | Int13n | tert-butyl (S)-6-methoxy- 7-(3-((2-(4-(((1- methylpyrrolidin-3- yl)oxy)carbonyl)piperidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.77 min, ES⁺ m/z 781.6 [M + H]⁺ | (500 MHz, DMSO-d₆) δ: 9.46 (d, J = 1.2 Hz, 1H), 9.23 (dd, J = 7.0, 1.5 Hz, 1H), 8.91 (s, 1H), 8.73 (dd, J = 4.0, 1.8 Hz, 1H), 8.44 (t, J = 5.8 Hz, 1H), 8.24 (d, J = 1.2 Hz, 1H), 7.17 (m, 1H), 6.88 (s, 1H), 6.49 (s, 1H), 6.38 (s, 1H), 5.04 (m, 1H), 4.14 (t, J = 4.3 Hz, 2H), 3.63 (s, 3H), 3.43 (q, J = 6.4 Hz, 2H), 3.39 (m, 2H), 2.85 (m, 2H), 2.61 (m, 2H), 2.52 (m, 1H, overlapped with DMSO), 2.47 (m, 2H, overlapped with DMSO), 2.26 (m, 2H), 2.22 (s, 3H), 2.15 (m, 1H), 2.04 (t, J = 9.9 Hz, 2H), 1.78 (m, 2H), 1.65 (m, 1H), 1.55 (m, 2H) | (Method 3): Rt = 3.45 min, ES⁺ m/z 681.3 [M + H]⁺ |
| | (S)-1-methylpyrrolidin-3-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 25 | 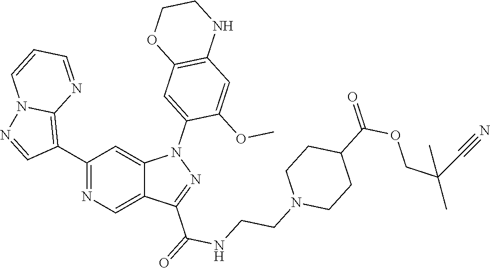 [SMILES: COc1cc2c(cc1-n1nc(C(=O)NCCN3CCC(C(=O)OCC(C)(C)C#N)CC3)c3cnc(-c4cnn5cccnc45)cc31)OCCN2] | Int 13o | tert-butyl 7-(3-((2-(4-((2- cyano-2- methylpropoxy)carbonyl) piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.92 min, ES⁺ m/z 779.5 [M + H]⁺ | (600 MHz, DMSO-d₆) δ: 9.46 (d, J = 1.1 Hz, 1H), 9.23 (dd, J = 7.0, 1.7 Hz, 1H), 8.91 (s, 1H), 8.74 (dd, J = 4.1, 1.7 Hz, 1H), 8.46 (t, J = 5.9 Hz, 1H), 8.24 (d, J = 1.3 Hz, 1H), 7.17 (m, 1H), 6.89 (s, 1H), 6.49 (s, 1H), 6.38 (s, 1H), 4.14 (t, J = 4.1 Hz, 2H), 4.08 (s, 2H), 3.63 (s, 3H), 3.44 (q, J = 6.6 Hz, 2H), 3.39 (m, 2H), 2.87 (br d, J = 11.4 Hz, 2H), 2.52 (m, overlapped with DMSO, 2H), 2.39- 2.45 (m, 1H), 2.05-2.13 (m, 2H), 1.82- 1.88 (m, 2H), 1.57-1.66 (m, 2H), 1.33 (m, 6H) | (Method 3): Rt = 4.32 min, ES⁺ m/z 679.3 [M + H]⁺ |
| | 2-cyano-2-methylpropyl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 26 | 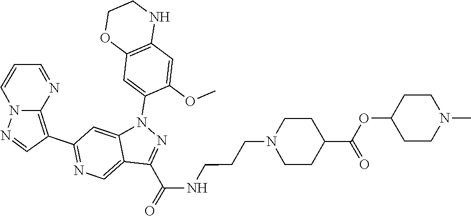 [SMILES: COc1cc2c(cc1-n1nc(C(=O)NCCCN3CCC(C(=O)OC4CCN(C)CC4)CC3)c3cnc(-c4cnn5cccnc45)cc31)OCCN2] | Int 13ai | tert-butyl 6-methoxy-7- (3-((3-(4-(((1- methylpiperidin-4- yl)oxy)carbonyl)piperidin- 1- yl)propyl)carbamoyl)-6- (pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 6): Rt = 0.60 min, ES⁺ m/z 809.5 [M + H]⁺ | (400 MHz, DMSO-d6) δ 9.42 (s, 1H), 9.20 (dd, 1H, J = 1.5, 7.0 Hz), 8.88 (s, 1H), 8.70 (br dd, 2H, J = 1.4, 4.1 Hz), 8.21 (s, 1H), 7.14 (dd, 1H, J = 4.2, 7.0 Hz), 6.84 (s, 1H), 6.45 (s, 1H), 6.34 (s, 1H), 4.64 (br s, 1H), 4.1-4.1 (s, 2H), 3.7-3.59 (m, 4H), 3.3-3.4 (m, 4H), 1.3- 2.9 (m, 20H) | (Method 3): Rt = 4.47 min, ES⁺ m/z 709.2 [M + H]⁺ |
| | 1-methylpiperidin-4-yl 1-(3-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)propyl)piperidine-4- | | | | |
| | carboxylate | | | | |
| Example 27 | 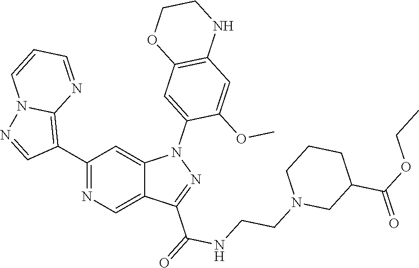 [SMILES: CCOC(=O)C1CCCN(CCNC(=O)c2nn(-c3cc4c(cc3OC)NCCO4)c3cc(-c4cnn5cccnc45)ncc23)C1] | Int13r | tert-butyl 7-(3-((2-(3- (ethoxycarbonyl)piperidin- 1-yl)ethyl)carbamoyl)- 6-(pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.94 min, ES⁺ m/z 726.5 [M + H]⁺ | (500 MHz, DMSO-d₆) δ: 9.46 (s, 1H), 9.24 (dd, J = 6.9, 1.6 Hz, 1H), 8.91 (s, 1H), 8.74 (dd, J = 3.9, 1.7 Hz, 1H), 8.43 (t, J = 5.7 Hz, 1H), 8.24 (s, 1H), 7.17 (dd, J = 6.9, 3.8 Hz, 1H), 6.88 (s, 1H), 6.49 (s, 1H), 6.37-6.42 (m, 1H), 4.13 (t, J = 4.3 Hz, 2H), 4.03 (q, J = 6.8 Hz, 2H), 3.63 (s, 3H), 3.41-3.48 (m, 2H), 3.37-3.41 (m, 2H), 2.82-2.88 (m, 1H), 2.65-2.71 (m, 1H), 2.51-2.55 (m, 3H), 2.25-2.34 (m, 1H), 2.08-2.18 (m, 1H), 1.72-1.80 (m, 1H), 1.60-1.68 (m, 1H), 1.37-1.51 (m, 2H), 1.18 (t, J = 7.0 Hz, 3H) | (Method 3): Rt = 4.16 min, ES⁺ m/z 626.3 [M + H]⁺ |
| | ethyl 1-(2-(1-(6-methoxy-3,4-dihydro- | | | | |
| | 2H-benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-3- | | | | |
| | carboxylate | | | | |
| Example 28 | 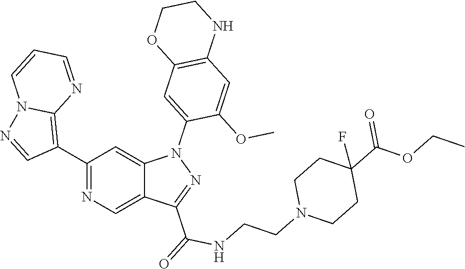 [SMILES: CCOC(=O)C1(F)CCN(CCNC(=O)c2nn(-c3cc4c(cc3OC)NCCO4)c3cc(-c4cnn5cccnc45)ncc23)CC1] | Int 13s | tert-butyl 7-(3-((2-(4- (ethoxycarbonyl)-4- fluoropiperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.89 min, ES⁺ m/z 744.4 [M + H]⁺ | (600 MHz, DMSO-d₆) δ: 9.45-9.47 (m, 1H), 9.24 (dd, J = 7.0, 1.8 Hz, 1H), 8.91 (s, 1H), 8.74 (dd, J = 4.0, 1.8 Hz, 1H), 8.50 (t, J = 5.7 Hz, 1H), 8.25 (d, J = 1.3 Hz, 1H), 7.15-7.20 (m, 1H), 6.89 (s, 1H), 6.49 (s, 1H), 6.39 (s, 1H), 4.11- 4.20 (m, 4H), 3.64 (s, 3H), 3.46 (q, J = 6.6 Hz, 2H), 3.37-3.42 (m, 2H), 2.78- 2.87 (m, 2H), 2.56 (t, J = 6.9 Hz, 2H), 2.25 (br t, J = 10.9 Hz, 2H), 1.92-2.07 (m, 2H), 1.83-1.92 (m, 2H), 1.21 (t, J = 7.1 Hz, 3H) | (Method 3): Rt = 4.18 min, ES⁺ m/z 644.3 [M + H]⁺ |
| | ethyl 4-fluoro-1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 29 | 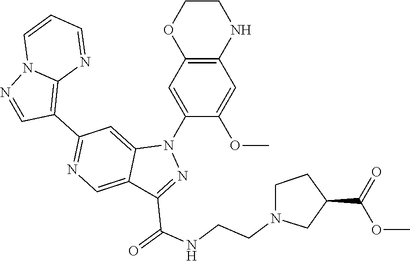 [SMILES: COC(=O)[C@@H]1CCN(CCNC(=O)c2nn(-c3cc4c(cc3OC)NCCO4)c3cc(-c4cnn5cccnc45)ncc23)C1] | Int 13t | tert-butyl (R)-6- methoxy-7-(3-((2-(3- (methoxycarbonyl)pyrrol idin-1- yl)ethyl)carbamoyl)-6- (pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.85 min, ES⁺ m/z 698.4 [M + H]⁺ | (500 MHz, DMSO-d₆) δ: 9.46 (d, J = 1.2 Hz, 1H), 9.2 (dd, J = 6.9, 1.8 Hz, 1H), 8.91 (s, 1H), 8.74 (dd, J = 4.1, 1.6 Hz, 1H), 8.45-8.49 (m, 1H), 8.24 (d, J = 1.1 Hz, 1H), 7.17 (dd, J = 6.9, 4.2 Hz, 1H), 6.89 (s, 1H), 6.49 (s, 1H), 6.37-6.39 (m, 1H), 4.13 (t, J = 4.5 Hz, 2H), 3.63 (s, 3H), 3.60 (s, 3H), 3.44 (q, J = 6.6 Hz, 2H), 3.37-3.40 (m, 2H), 2.99-3.05 (m, 1H), 2.83-2.89 (m, 1H), 2.59-2.67 (m, 3H), 2.50-2.53 (m, 2H, overlapped with DMSO); 1.91-2.01 (m, 2H) | (Method 3): Rt = 3.85 min, ES⁺ m/z 598.2 [M + H]⁺ |
| | methyl (R)-1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)pyrrolidine-3- | | | | |
| | carboxylate | | | | |
| Example 30 | 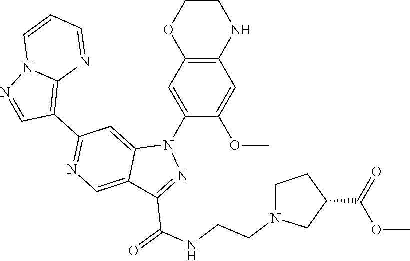 [SMILES: COC(=O)[C@H]1CCN(CCNC(=O)c2nn(-c3cc4c(cc3OC)NCCO4)c3cc(-c4cnn5cccnc45)ncc23)C1] | Int 13u | tert-butyl (S)-6-methoxy- 7-(3-((2-(3- (methoxycarbonyl) pyrrolidin-1- yl)ethyl)carbamoyl)-6- (pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.87 min, ES⁺ m/z 698.4 [M + H]⁺ | (500 MHz, DMSO-d₆) δ: 9.46 (d, J = 1.2 Hz, 1H), 9.23 (dd, J = 6.9, 1.8 Hz, 1H), 8.91 (s, 1H), 8.74 (dd, J = 4.1, 1.6 Hz, 1H), 8.47 (t, J = 5.7 Hz, 1H), 8.24 (d, J = 1.1 Hz, 1H), 7.17 (dd, J = 6.9, 4.2 Hz, 1H), 6.89 (s, 1H), 6.49 (s, 1H), 6.37-6.39 (m, 1H), 4.13 (t, J = 4.4 Hz, 2H), 3.63 (s, 3H), 3.60 (s, 3H), 3.44 (q, J = 6.6 Hz, 2H), 3.37-3.40 (m, 2H), 2.99-3.05 (m, 1H), 2.85 (t, J = 8.8 Hz, 1H), 2.59-2.67 (m, 3H), 2.50-2.55 (m, 2H, overlapped with DMSO), 1.91-2.01 (m, 2H) | (Method 3): Rt = 3.85 min, ES⁺ m/z 598.2 [M + H]⁺ |
| | methyl (S)-1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)pyrrolidine-3- | | | | |
| | carboxylate | | | | |
| Example 31 | 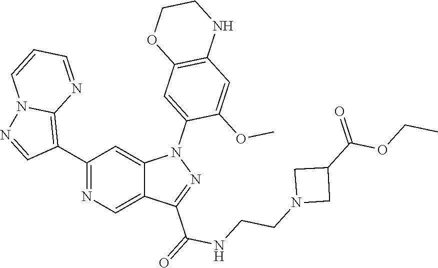 [SMILES: CCOC(=O)C1CN(CCNC(=O)c2nn(-c3cc4c(cc3OC)NCCO4)c3cc(-c4cnn5cccnc45)ncc23)C1] | Int 13v | tert-butyl 7-(3-((2-(3- (ethoxycarbonyl)azetidin- 1-yl)ethyl)carbamoyl)- 6-(pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.89 min, ES⁺ m/z 698.4 [M + H]⁺ | (500 MHz, DMSO-d₆) δ: 9.46 (d, J = 1.0 Hz, 1H), 9.24 (dd, J = 6.9, 1.6 Hz, 1H), 8.91 (s, 1H), 8.74 (dd, J = 4.2, 1.7 Hz, 1H), 8.43 (t, J = 5.9 Hz, 1H), 8.24 (d, J = 0.9 Hz, 1H), 7.17 (dd, J = 6.9, 4.0 Hz, 1H), 6.88 (s, 1H), 6.49 (s, 1H), 6.37- 6.42 (m, 1H), 4.13 (t, J = 4.1 Hz, 2H), 4.08 (q, J = 6.8 Hz, 2H), 3.63 (s, 3H), 3.43-3.49 (m, 2H), 3.37-3.41 (m, 2H), 3.25-3.30 (m, 2H), 3.16-3.12 (m, 2H), 2.55-2.60 (m, 2H), 2.50-2.53 (m, 1H), 1.18 (t, J = 7.0 Hz, 3H) | (Method 3): Rt = 3.96 min, ES⁺ m/z 598.2 [M + H]⁺ |
| | ethyl 1-(2-(1-(6-methoxy-3,4-dihydro- | | | | |
| | 2H-benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)azetidine-3- | | | | |
| | carboxylate | | | | |
| Example 32 | 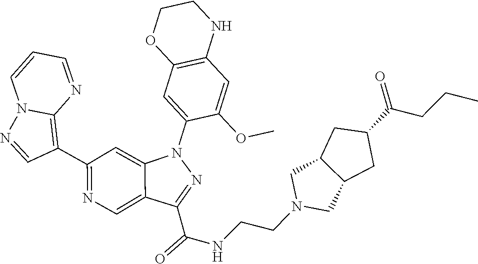 [SMILES: CCCC(=O)[C@@H]1C[C@@H]2CN(CCNC(=O)c3nn(-c4cc5c(cc4OC)NCCO5)c4cc(-c5cnn6cccnc56)ncc34)C[C@@H]2C1] | Int 13w | tert-butyl 7-(3-((2- ((3aR,51,6aS)-5- (ethoxycarbonyl)hexahydro- cyclopenta [c]pyrrol- 2 (1H)- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.91 min, ES⁺ m/z 752.4 [M + H]⁺ | (500 MHz, DMSO-d₆) δ: 9.46 (d, J = 1.1 Hz, 1H), 9.24 (dd, J = 7.0, 1.7 Hz, 1H), 8.92 (s, 1H), 8.74 (dd, J = 4.1, 1.7 Hz, 1H), 8.51 (t, J = 6.0 Hz, 1H), 8.25 (d, J = 1.2 Hz, 1H), 7.18 (dd, J = 7.1, 4.1 Hz, 1H), 6.89 (s, 1H), 6.50 (s, 1H), 6.39- 6.37 (m, 1H), 4.14 (t, J = 4.3 Hz, 2H), 3.99 (q, J = 7.1 Hz, 2H), 3. 64 (s, 3H), 3.44 (q, J = 6.6 Hz, 2H), 3.41-3.38 (m, 2H), 2.65 (d, J = 8.8 Hz, 2H), 2.61-2.53 (m, 3H), 2.50-2.46 (m, 2H, overlapped with DMSO), 2.22-2.16 (m, 2H), 2.10- 2-04 (m, 2H), 1.54-1.46 (m, 2H), 1.11 (t, J = 7.1 Hz, 3H) | (Method 3): Rt = 4.35 min, ES⁺ m/z 652.3 [M + H]⁺ |
| | ethyl (3aR,5r,6aS)-2-(2-(1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridine-3- | | | | |
| | carboxamido)ethyl)octahydrocyclopenta | | | | |
| | [c]pyrrole-5-carboxylate | | | | |
| Example 33 | 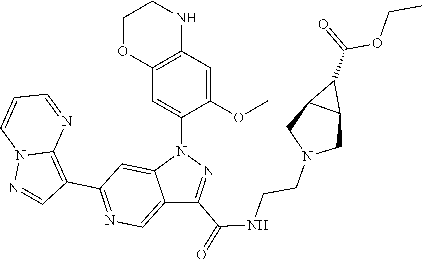 | Int 13x | tert-butyl 7-(3-((2- ((1R,5S,6r)-6- (ethoxycarbonyl)-3- azabicyclo[3.1.0]hexan- 3-yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 2): Rt = 1.32 min, ES⁺ m/z 724.4 [M + H]⁺ | (600 MHz, DMSO-d₆) δ: 9.46 (d, J = 1.1 Hz, 1H), 9.24 (dd, J = 7.1, 1.7 Hz, 1H), 8.92 (s, 1H), 8.74 (dd, J = 4.0, 1.7 Hz, 1H), 8.47 (t, J = 5.9 Hz, 1H), 8.25 (d, J = 1.1 Hz, 1H), 7.18 (dd, J = 7.0, 4.0 Hz, 1H), 6.90 (s, 1H), 6.50 (s, 1H), 6.39 (s, 1H), 4.15 (t, J = 4.4 Hz, 2H), 4.02 (q, J = 7.2 Hz, 2H), 3.64 (s, 3H), 3.37-3.42 (m, 4H), 3.12 (d, J = 9.0 Hz, 2H), 2.57- 2.65 (m, 2H), 2.39 (d, J = 9.0 Hz, 2H), 1.90 (s, 2H), 1.81-1.86 (m, 1H), 1.16 (t, J = 7.1 Hz, 3H). | (Method 3): Rt = 4.12 min, ES⁺ m/z 624.3 [M + H]⁺ |
| | ethyl (1R,5S,6r)-3-(2-(1-(6-methoxy- | | | | |
| | 3,4-dihydro-2H-benzo[b][1,4]oxazin-7- | | | | |
| | yl)-6-(pyrazolo[1,5-a]pyrimidin-3-yl)- | | | | |
| | 1H-pyrazolo[4,3-c]pyridine-3- | | | | |
| | carboxamido)ethyl)-3- | | | | |
| | azabicyclo[3.1.0]hexane-6-carboxylate | | | | |
| Example 34 | 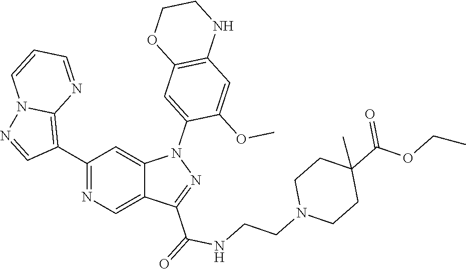 [SMILES: CCOC(=O)C1(C)CCN(CCNC(=O)c2nn(-c3cc4c(cc3OC)NCCO4)c3cc(-c4cnn5cccnc45)ncc23)CC1] | Int 13y | tert-butyl 7-(3-((2-(4- (ethoxycarbonyl)-4- methylpiperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.96 min, ES⁺ m/z 740.5 [M + H]⁺ | (500 MHz, DMSO-d₆) δ: 9.46 (s, 1H), 9.23 (d, J = 6.7 Hz, 1H), 8.91 (s, 1H), 8.75-8.72 (m, 1H), 8.44 (br d, J = 1.8 Hz, 1H), 8.24 (s, 1H), 7.17 (dd, J = 4.1, 6.9 Hz, 1H), 6.89 (s, 1H), 6.49 (s, 1H), 6.39 (br s, 1H), 4.14 (br t, J = 3.8 Hz, 2H), 4.09 (q, J = 7.0 Hz, 2H), 3.63 (s, 3H), 3.44 (br d, J = 5.5 Hz, 2H), 3.39 (br s, 2H), 2.72-2.63 (m, 2H), 2.5 (ov with DMSO, 2H) 2.16-2.05 (m, 2H), 1.98 (br d, J = 12.2 Hz, 2H), 1.42 (br t, J = 10.2 Hz, 2H), 1.18 (t, J = 7.0 Hz, 3H), 1.11 (s, 3H) | (Method 3): Rt = 4.55 min, ES⁺ m/z 640.3 [M + H]⁺ |
| | ethyl 1-(2-(1-(6-methoxy-3,4-dihydro- | | | | |
| | 2H-benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)-4- | | | | |
| | methylpiperidine-4-carboxylate | | | | |
| Example 35 | 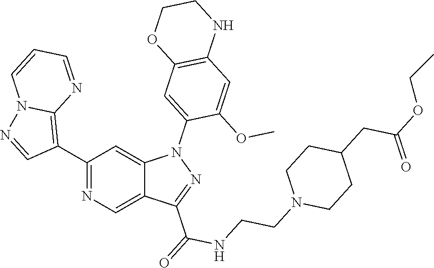 [SMILES: CCOC(=O)CC1CCN(CCNC(=O)c2nn(-c3cc4c(cc3OC)NCCO4)c3cc(-c4cnn5cccnc45)ncc23)CC1] | Int 13z | tert-butyl 7-(3-((2-(4-(2- ethoxy-2- oxoethyl)piperidin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.91 min, ES⁺ m/z 740.5 [M + H]⁺ | (600 MHz, DMSO-d₆) δ: 9.46 (d, J = 1.1 Hz, 1H), 9.24 (dd, J = 6.9, 1.6 Hz, 1H), 8.91 (s, 1H), 8.74 (dd, J = 4.1, 1.5 Hz, 1H), 8.42 (t, J = 4.6 Hz, 1H), 8.25 (d, J = 1.1 Hz, 1H), 7.17 (dd, J = 7.2, 4.1 Hz, 1H), 6.88 (s, 1H), 6.49 (s, 1H), 6.39 (bs, 1H), 4.14 (t, J = 4.2 Hz, 2H), 4.04 (q, J = 7.1 Hz, 2H), 3.63 (s, 3H), 3.43 (q, J = 6.4 Hz, 2H), 3.41-3.38 (m, 2H), 2.92-2.87 (m, 2H), 2.53-2.50 (m, 2H, overlapped with DMSO), 2.21 Hz (d, J = 6.6 Hz, 2H), 1.98-1.92 (m, 2H), 1.68- 1.62 (m, 1H), 1.64-1.59 (m, 2H), 1.22- 1.18 (m, 2H), 1.17 (t, J = 7.3 Hz, 3H) | (Method 3): Rt = 4.24 min, ES⁺ m/z 640.3 [M + H]⁺ |
| | ethyl 2-(1-(2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)piperidin-4- | | | | |
| | yl)acetate | | | | |
| Example 36 | 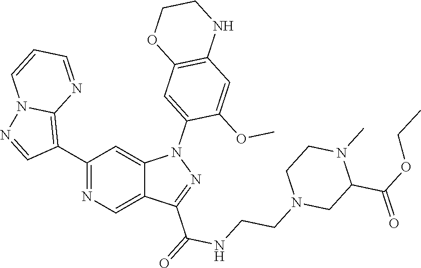 [SMILES: CCOC(=O)C1CN(CCNC(=O)c2nn(-c3cc4c(cc3OC)NCCO4)c3cc(-c4cnn5cccnc45)ncc23)CCN1C] | Int 13aa | tert-butyl 7-(3-((2-(3- (ethoxycarbonyl)-4- methylpiperazin-1- yl)ethyl)carbamoyl)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.90 min, ES⁺ m/z 741.5 [M + H]⁺ | (500 MHz, DMSO-d₆) δ: 9.46 (d, J = 0.9 Hz, 1H), 9.23 (dd, J = 7.0, 1.5 Hz, 1H), 8.91 (s, 1H), 8.73 (dd, J = 4.1, 1.7 Hz, 1H), 8.36 (br t, J = 5.5 Hz, 1H), 8.24 (d, J = 0.9 Hz, 1H), 7.17 (dd, J = 6.9, 4.1 Hz, 1H), 6.88 (s, 1H), 6.49 (s, 1H), 6.38 (s, 1H), 4.14 (t, J = 4.3 Hz, 2H), 4.10-3.99 (m, 2H), 3.63 (s, 3H), 3.48-3.37 (m, 4H), 3.04-2.95 (m, 1H), 2.93-2.84 (m, 1H), 2.74-2.66 (m, 1H), 2.62-2.51 (overlapping with DMSO, 4H), 2.42- 2.35 (m, 1H), 2.23 (m, 1H), 2.23 (s, 3H), 1.13 (t, J = 7.0 Hz, 3H) | (Method 3): Rt = 4.03 min, ES⁺ m/z 641.3 [M + H]⁺ |
| | ethyl 4-(2-(1-(6-methoxy-3,4-dihydro- | | | | |
| | 2H-benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)-1- | | | | |
| | methylpiperazine-2-carboxylate | | | | |
| Example 37 | 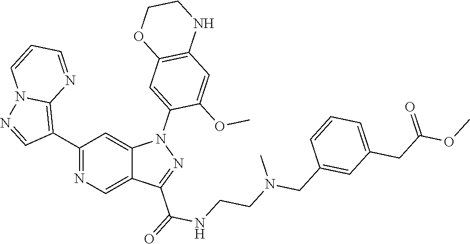 [SMILES: COC(=O)Cc1cccc(CN(C)CCNC(=O)c2nn(-c3cc4c(cc3OC)NCCO4)c3cc(-c4cnn5cccnc45)ncc23)c1] | Int 13ab | tert-butyl 6-methoxy-7- (3-((2-((3-(2-methoxy-2- oxoethyl)benzyl)(methyl) amino)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 1): Rt = 0.96 min, ES⁺ m/z 762.4 [M + H]⁺ | (600 MHz, DMSO-d₆) δ: 9.45 (m, 1H), 9.23 (dd, J = 7.0, 1.7 Hz, 1H), 8.91 (s, 1H), 8.74 (dd, J = 4.1, 1.7 Hz, 1H), 8.41 (br t, J = 5.6 Hz, 1H), 8.25 (d, J = 1.1 Hz, 1H), 7.14-7.23 (m, 4H), 7.09 (m, 1H), 6.89 (s, 1H), 6.49 (s, 1H), 6.39 (s, 1H), 4.13 (t, J = 4.3 Hz, 2H), 3.63 (s, 3H), 3.56 (m, 5H), 3.52 (s, 2H), 3.47 (m, 2H), 3.28 (m, 2H), 2.56 (m, overlapped with DMSO, 2H), 2.19 (s, 3H) | (Method 3): Rt = 4.39 min, ES⁺ m/z 662.3 [M + H]⁺ |
| | methyl 2-(3-(((2-(1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridine-3- | | | | |
| | carboxamido)ethyl)(methyl)amino) | | | | |
| | methyl)phenyl)acetate | | | | |
| Example 38 | 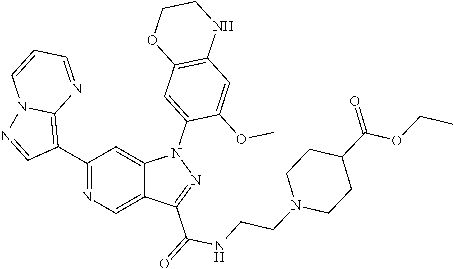 [SMILES: CCOC(=O)C1CCN(CCNC(=O)c2nn(-c3cc4c(cc3OC)NCCO4)c3cc(-c4cnn5cccnc45)ncc23)CC1] | Int 11b | tert-butyl 6-(3-((2-(4- (ethoxycarbonyl)piperidin- 1-yl)ethyl)carbamoyl)- 6-(pyrazolo[1,5- a]pyrimidin-3-yl)-1H- pyrazolo[4,3-c]pyridin-1- yl)-7-methoxy-3,4- dihydroquinoline-1 (2H)- carboxylate (Method 1): Rt = 0.91 min, ES⁺ m/z 724.3 [M + H]⁺ | (500 MHz, DMSO-d₆) δ: 9.46 (d, J = 1.1 Hz, 1H), 9.23 (dd, J = 7.0 Hz, J = 1.7 Hz, 1H), 8.91 (s, 1H), 8.74 (dd, J = 4.0 Hz, J = 1.7 Hz, 1H), 8.45 (m, 1H), 8.23 (d, J = 1.1 Hz, 1H), 7.17 (dd, J = 7.0 Hz, J = 4.0 Hz, 1H), 7.01 (s, 1H), 6.33 (s, 1H), 6.26 (s, 1H), 4.05 (q, J = 7.1 Hz, 2H), 3.63 (s, 3H), 3.44 (br d, J = 5.1 Hz, 2H), 3.27 (m, 2H), 2.86 (m, 2H), 2.66 (t, J = 6.1 Hz, 2H), 2.51-2.54 (m, overlapped with DMSO, 2H), 2.28 (m, 1H), 2.04 (m, 2H), 1.82 (m, 4H), 1.56 (m, 2H), 1.17 (t, J = 7.2 Hz, 3H) | (Method 3): Rt = 4.44 min, ES⁺ m/z 624.3 [M + H]⁺ |
| | ethyl 1-(2-(1-(7-methoxy-1,2,3,4- | | | | |
| | tetrahydroquinolin-6-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |
| Example 39 | 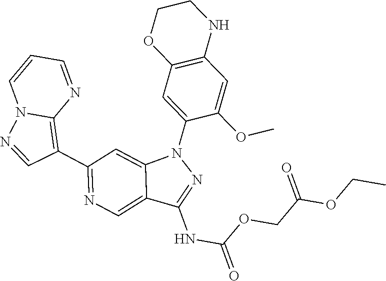 [SMILES: CCOC(=O)COC(=O)Nc1nn(-c2cc3c(cc2OC)NCCO3)c2cc(-c3cnn4cccnc34)ncc12] | Int 14a | tert-butyl 7-(3-(((2- ethoxy-2- oxoethoxy)carbonyl) amino)-6-(pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 1.03 min, ES⁺ m/z 645.2 [[M + H]⁺ | (300 MHz, DMSO-d₆) δ: 10.76 (br s, 1H), 9.21 (dd, J = 7.1, 1.7 Hz, 1H), 9.19 (d, J = 0.9 Hz, 1H), 8.86 (s, 1H), 8.71 (dd, J = 4.1, 1.7 Hz, 1H), 8.09 (d, J = 0.9 Hz, 1H), 7.15 (dd, J = 7.0, 4.0 Hz, 1H), 6.75 (s, 1H), 6.46 (s, 1H), 6.24-6.33 (m, 1H), 4.77 (s, 2H), 4.19 (q, J = 7.1 Hz, 2H), 4.07-4.15 (m, 2H), 3.62 (s, 3H), 3.32-3.42 (m, 2H), 1.23 (t, J = 7.1 Hz, 3H) | (Method 3): Rt = 4.53 min, ES⁺ m/z 545.2 [M + H]⁺ |
| | ethyl 2-(((1-(6-methoxy-3,4-dihydro- | | | | |
| | 2H-benzo[b][1,4]oxazin-7-yl)-6- | | | | |
| | (pyrazolo[1,5-a]pyrimidin-3-yl)-1H- | | | | |
| | pyrazolo[4,3-c]pyridin-3- | | | | |
| | yl)carbamoyl)oxy)acetate | | | | |
| Example 40 | 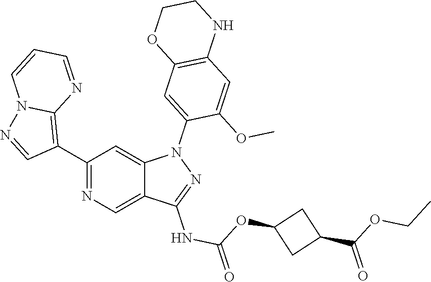 [SMILES: CCOC(=O)[C@H]1C[C@@H](OC(=O)Nc2nn(-c3cc4c(cc3OC)NCCO4)c3cc(-c4cnn5cccnc45)ncc23)C1] | Int 14b | tert-butyl 7-(3-((((1s,3s)- 3- (ethoxycarbonyl)cyclo- butoxy)carbonyl)amino)-6- (pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 1.04 min, ES⁺ m/z 685.3 [M + H]⁺ | (600 MHz, DMSO-d₆) δ: 10.48 (br s, 1H), 9.20-9.24 (m, 2H), 8.87 (s, 1H), 8.71 (dd, J = 4.0 Hz, J = 1.5 Hz, 1H), 8.09 (s, 1H), 7.15 (dd, J = 7.0 Hz, J = 4.0 Hz, 1H), 6.74 (s, 1H), 6.47 (s, 1H), 6.28 (s, 1H), 4.98-5.04 (m, 1H), 4.07-4.12 (m, 4H), 3.63 (s, 3H), 3.37 (m, 2H), 2.82- 2.90 (m, 1H), 2.62-2.68 (m, 2H), 2.23- 2.30 (m, 2H), 1.20 (t, J = 7.0 Hz, 3H) | (Method 3): Rt = 4.96 min, ES⁺ m/z 585.2 [M + H]⁺ |
| | ethyl (1s,3s)-3-(((1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridin-3- | | | | |
| | yl)carbamoyl)oxy)cyclobutane-1- | | | | |
| | carboxylate | | | | |
| Example 41 | 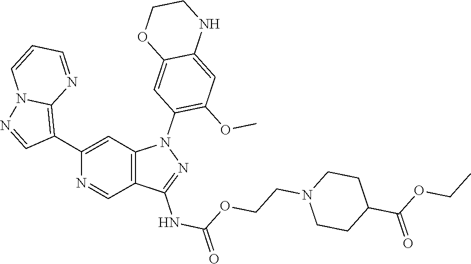 [SMILES: CCOC(=O)C1CCN(CCOC(=O)Nc2nn(-c3cc4c(cc3OC)NCCO4)c3cc(-c4cnn5cccnc45)ncc23)CC1] | Int14c | tert-butyl 7-(3-(((2-(4- (ethoxycarbonyl)piperidin- 1- yl)ethoxy)carbonyl)amino)- 6-(pyrazolo[1,5- a]pyrimidin-3-yl)-1H- pyrazolo[4,3-c]pyridin-1- yl)-6-methoxy-2,3- dihydro-4H- benzo[b][1,4]oxazine-4- carboxylate (Method 1): Rt = 0.85 min, ES⁺ m/z 742.4 [[M + H]⁺ | (500 MHz, DMSO-d₆) δ: 10.41 (bs, 1H), 9.27 (s, 1H), 9.23 (dd, J = 7.0, 1.6 Hz, 1H), 8.87 (s, 1H), 8.72 (dd, J = 4.1, 1.6 Hz, 1H), 8.09 (s, 1H), 7.15 (dd, J = 6.9, 4.0 Hz, 1H), 6.73 (s, 1H), 6.47 (s, 1H), 6.28 (bs, 1H), 4.27 (t, J = 4.8 Hz, 2H), 4.13 (t, J = 4.2 Hz, 2H), 4.06 (q, J = 7.1 Hz, 2H), 3.62 (s, 3H), 3.39-3.35 (m, 2H), 2.91-2.85 (m, 2H), 2.61 (t, J = 5.1 Hz, 2H), 2.33-2.24 (m, 1H), 2.08 (t, J = 11.5 Hz, 2H), 1.84-1.77 (m, 2H), 1.67-1.56 (m, 2H), 1.17 (t, J = 7.1 Hz, 3H) | (Method 3): Rt = 3.79 min, ES⁺ m/z 642.3 [M + H]⁺ |
| | ethyl 1-(2-(((1-(6-methoxy-3,4- | | | | |
| | dihydro-2H-benzo[b][1,4]oxazin-7-yl)- | | | | |
| | 6-(pyrazolo[1,5-a]pyrimidin-3-yl)-1H- | | | | |
| | pyrazolo[4,3-c]pyridin-3- | | | | |
| | yl)carbamoyl)oxy)ethyl)piperidine-4- | | | | |
| | carboxylate | | | | |
| Example 42 | 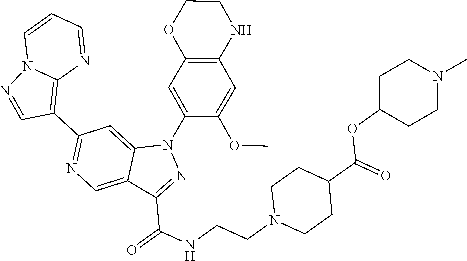 [SMILES: COc1cc2c(cc1-n1nc(C(=O)NCCN3CCC(C(=O)OC4CCN(C)CC4)CC3)c3cnc(-c4cnn5cccnc45)cc31)OCCN2] | Int 13ah | tert-butyl 6-methoxy-7- (3-((2-(4-(((1- methylpiperidin-4- yl)oxy)carbonyl)piperidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate (Method 6): Rt = 0.56 min, ES⁺ m/z 795.4 [[M + H]⁺ | (400 MHz, DMSO-d6) δ 9.42 (d, 1H, J = 1.1 Hz), 9.20 (dd, 1H, J = 1.8, 7.0 Hz), 8.88 (s, 1H), 8.70 (dd, 1H, J = 1.8, 3.9 Hz), 8.42 (t, 1H, J = 5.8 Hz), 8.21 (d, 1H, J = 1.1 Hz), 7.14 (dd, 1H, J = 4.1, 6.9 Hz), 6.85 (s, 1H), 6.46 (s, 1H), 6.35 (s, 1H), 4.6-4.7 (m, 1H), 4.10 (t, 2H, J = 4.1 Hz), 3.60 (s, 3H), 3.3-3.4 (m, 4H), 2.8-2.9 (m, 2H), 1.3-2.4 (m, 20H) | (Method 3): Rt = 4.50 min, ES⁺ m/z 695.4 [M + H]⁺ |
| | 1-methylpiperidin-4-yl 1-(2-(1-(6- | | | | |
| | methoxy-3,4-dihydro-2H- | | | | |
| | benzo[b][1,4]oxazin-7-yl)-6- | | | | |
| | (pyrazolo[1,5-a]pyrimidin-3-yl)-1H- | | | | |
| | pyrazolo[4,3-c]pyridine-3- | | | | |
| | carboxamido)ethyl)piperidine-4- | | | | |
| | carboxylate | | | | |

### Example 43

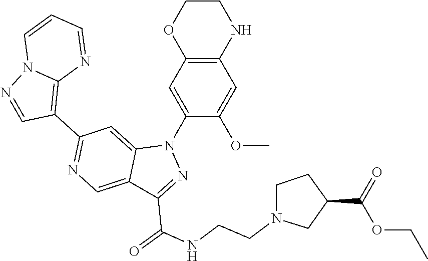 [SMILES: CCOC(=O)[C@@H]1CCN(CCNC(=O)c2nn(-c3cc4c(cc3OC)NCCO4)c3cc(-c4cnn5cccnc45)ncc23)C1]

### Ethyl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate (Example 43)

A solution of example 29 (38.0 mg, 0.0636 mmol) and concentrated sulfuric acid (170 μL, 3.18 mmol) in EtOH (1 mL) was stirred at 80° C. for 4 h. After cooling to RT, RM was diluted with sat.aq. NaHCO₃, DCM (5 mL) and the pH adjusted to 9.5. Aqueous layer was extracted with DCM/i-PrOH (3:1; 4×5 mL) and the combined organic layers were passed through a phase separator and solvents removed in vacuo. The residue was purified by flash chromatography on Si cartridge by eluting with 0-70% DCM/MeOH/NH₄OH (90:5:0.5) in DCM to afford the title product (28 mg).

LCMS (Method 3), Rt=4.05 min, ES⁺ m/z 612.3 [M+H]⁺

¹H-NMR (500 MHz, DMSO-d₆) δ: 9.46 (d, J=1.2 Hz, 1H), 9.23 (dd, J=6.9, 1.8 Hz, 1H), 8.91 (s, 1H), 8.74 (dd, J=4.1, 1.6 Hz, 1H), 8.46 (t, J=6.0 Hz 1H), 8.24 (d, J=1.1 Hz, 1H), 7.17 (dd, J=6.9, 4.1 Hz, 1H), 6.89 (s, 1H), 6.49 (s, 1H), 6.37-6.39 (m, 1H), 4.14 (t, J=4.5 Hz, 2H), 4.06 (q, J=7.2 Hz, 2H), 3.63 (s, 3H), 3.44 (q, J=6.3 Hz, 2H), 3.37-3.40 (m, 2H), 2.97-3.03 (m, 1H), 2.85 (t, J=8.7 Hz, 1H), 2.59-2.65 (m, 3H), 2.50-2.53 (m, 2H, overlapped with DMSO), 1.91-2.01 (m, 2H), 1.17 (t, J=7.1 Hz, 3H).

### Examples 44 to 45

The following Examples were prepared in a similar manner to example 43 from the indicated starting materials.

| Example | Structure/name | Intermediate | ¹H-NMR | LCMS |
|---|---|---|---|---|
| Example 44 | 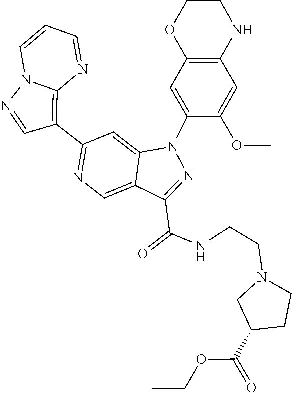 [SMILES: CCOC(=O)[C@H]1CCN(CCNC(=O)c2nn(-c3cc4c(cc3OC)NCCO4)c3cc(-c4cnn5cccnc45)ncc23)C1] | Example 30 and ethanol | (600 MHz, DMSO-d₆) δ: 9.46 (d, J = 1.2 Hz, 1H), 9.24 (dd, J = 6.9, 1.8 Hz, 1H), 8.91 (s, 1H), 8.74 (dd, J = 4.1, 1.7 Hz, 1H), 8.46 (t, J = 6.0 Hz 1H), 8.24 (d, J = 1.1 Hz, 1H), 7.17 (dd, J = 6.9, 4.1 Hz, 1H), 6.89 (s, 1H), 6.49 (s, 1H), 6.37-6.40 (m, 1H), 4.14 (t, J = 4.6 Hz, 2H), 4.06 (q, J = 7.2 Hz, 2H), 3.63 (s, 3H), 3.44 (q, J = 6.5 Hz, 2H), 3.37-3.41 (m, 2H), 2.97-3.03 (m, 1H), 2.85 (t, J = 8.7 Hz, 1H), 2.59-2.65 (m, 3H), 2.50-2.53 (m, 2H, overlapped with DMSO); 1.91-2.02 (m, 2H), 1.17 (t, J = 7.1 Hz, 3H) | (Method 3), Rt = 4.05 min, ES⁺ m/z 612.3 [M + H]⁺ |
| | ethyl (S)-1-(2-(1-(6- | | | |
| | methoxy-3,4-dihydro- | | | |
| | 2H- | | | |
| | benzo[b][1,4]oxazin-7- | | | |
| | yl)-6-(pyrazolo[1,5- | | | |
| | a]pyrimidin-3-yl)-1H- | | | |
| | pyrazolo[4,3- | | | |
| | c]pyridine-3- | | | |
| | carboxamido)ethyl) | | | |
| | pyrrolidine-3-carboxylate | | | |
| Example 45 | 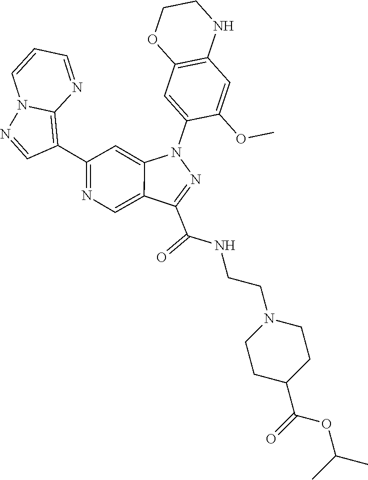 [SMILES: COc1cc2c(cc1-n1nc(C(=O)NCCN3CCC(C(=O)OC(C)C)CC3)c3cnc(-c4cnn5cccnc45)cc31)OCCN2] | Example 9 and isopropanol | (500 MHz, DMSO-d₆) δ: 9.46 (s, 1H), 9.23 (dd, J = 7.0, 1.5 Hz, 1H), 8.91 (s, 1H), 8.74 (dd, J = 4.0, 1.5 Hz, 1H), 8.44 (t, J = 5.4 Hz, 1H), 8.24 (s, 1H), 7.17 (dd, J = 7.0, 4.0 Hz, 1H), 6.88 (s, 1H), 6.38 (s, 1H), 4.84-4.92 (m, 1H), 4.14 (t, J = 4.1 Hz, 2H), 3.63 (s, 3H), 3.44 (q, J = 6.0 Hz, 2H), 3.37-3.40 (m, 2H), 2.81- 2.89 (m, 2H), 2.50 (m, 2H, overlapped with DMSO), 2.20-2.28 (m, 1H), 2.02-2.09 (m, 2H), 1.76-1.78 (m, 2H), 1.59- 1.51 (m, 2H), 1.17 (d, J = 6.41 Hz, 6H) | (Method 3): Rt = 4.45 min, ES⁺ m/z 640.3 [M + H]⁺ |
| | isopropyl 1-(2-(1-(6- | | | |
| | methoxy-3,4-dihydro- | | | |
| | 2H- | | | |
| | benzo[b][1,4]oxazin-7- | | | |
| | yl)-6-(pyrazolo[1,5- | | | |
| | a]pyrimidin-3-yl)-1H- | | | |
| | pyrazolo[4,3- | | | |
| | c]pyridine-3- | | | |
| | carboxamido)ethyl) | | | |
| | piperidine-4-carboxylate | | | |

### Example 46

### Step 1

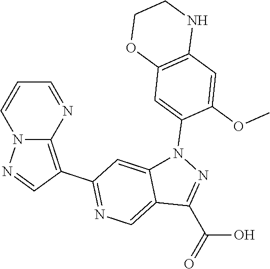 [SMILES: COc1cc2c(cc1-n1nc(C(=O)O)c3cnc(-c4cnn5cccnc45)cc31)OCCN2]

### 1-(6-Methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylic acid (Example 46-intermediate 1)

A degassed mixture of intermediate 8 (50 mg, 0.14 mmol), 3-(4,4,5,5-tetramethyl-1,3,2-dioxaborolan-2-yl)pyrazolo[1,5-a]pyrimidine (47.6 mg, 0.19 mmol), K₃PO₄ (58.8 mg, 0.28 mmol) and XPhos PdG3 (5.9 mg, 7 μmol) in 1/1 water/THF (5 mL) was stirred under nitrogen at 70° C. for 1.5 h. After cooling to RT, solvent was removed in vacuo. The residue was suspended in 1M aq. HCl to form a precipitate that was collected by filtration, followed by a washing with water. The filtrate was additionally extracted with EtOAc (3×20 mL), DCM (2×10 mL) and DCM: i-PrOH (1:1, 2×20 mL). The combined organic layers were dried over Na₂SO₄ and solvents removed in vacuo. Combined crude materials were purified by flash chromatography on Si cartridge by eluting with 0-100% DCM/MeOH/AcOH (90:9:1.5) in DCM to afford the title product (37.9 mg).

LCMS (Method 3), Rt=3.79 min, ES⁺ m/z 444.1 [M+H]⁺

### Step 2

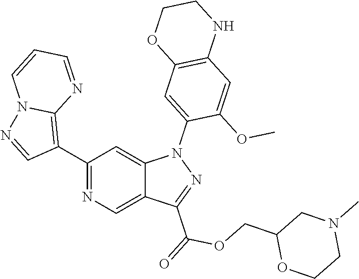 [SMILES: COc1cc2c(cc1-n1nc(C(=O)OCC3CN(C)CCO3)c3cnc(-c4cnn5cccnc45)cc31)OCCN2]

### (4-Methylmorpholin-2-yl)methyl 1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylate (Example 46)

A solution of example 46-intermediate 1 (20.0 mg, 0.04 mmol), (4-methylmorpholin-2-yl)methanol (18.5 mg, 0.14 mmol), EDC*HCl (13.5 mg, 0.07 mmol) and DMAP (4.3 mg, 35 μmol) in DMF (1 mL) was stirred at RT overnight. RM was partitioned between EtOAc (10 mL) and water (5 mL). The organic layer was washed with sat. aq. NaCl (5 mL) and solvent removed in vacuo. The residue was purified by flash chromatography on Si cartridge by eluting with 0-20% DCM/MeOH/NH₄OH (90:9:0.5) in DCM to afford the title product (6.6 mg).

LCMS (Method 3), Rt=4.21 min, ES⁺ m/z 557.2 [M+H]⁺

¹H-NMR (500 MHz, DMSO-d₆) δ: 9.40 (s, 1H), 9.26-9.22 (m, 1H), 8.92 (s, 1H), 8.76-8.73 (m, 1H), 8.28 (s, 1H), 7.18 (dd, J=4.3, 7.0 Hz, 1H), 6.87 (s, 1H), 6.51 (s, 1H), 6.43 (s, 1H), 4.46-4.41 (m, 2H), 4.15 (t, J=4.1 Hz, 2H), 3.92-3.83 (m, 2H), 3.63 (s, 3H), 3.61-3.56 (m, 1H), 3.40 (br d, J=1.5 Hz, 2H), 2.80 (br d, J=11.3 Hz, 1H), 2.62 (br d, J=11.9 Hz, 1H), 2.21 (s, 3H), 2.09-2.03 (m, 1H), 1.93 (t, J=10.7 Hz, 1H)

### Example 47

### Step 1

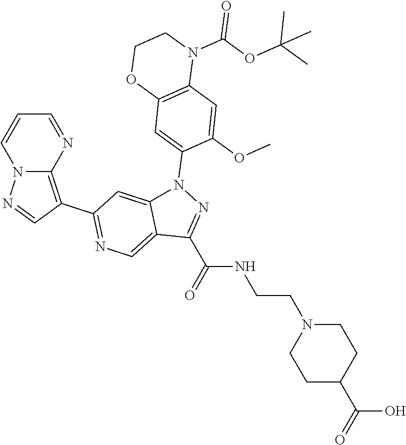 [SMILES: COc1cc2c(cc1-n1nc(C(=O)NCCN3CCC(C(=O)O)CC3)c3cnc(-c4cnn5cccnc45)cc31)OCCN2C(=O)OC(C)(C)C]

### 1-(2-(1-(4-(tert-butoxycarbonyl)-6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylic acid (Example 47-intermediate 1)

A mixture of Example 9-intermediate 1 (1.0 g, 1.38 mmol) and LiOH (1.0 M in water, 6.9 mL, 6.9 mmol) in THF (15 mL) was stirred at RT overnight. RM was diluted with water and washed with EtOAc. After pH acidification of aqueous layer to 5 by using aq. 1.0 M HCl and cooling on ice for 30 min, the formed precipitate was filtered, washed with water several times and dried to afford the title compound (842 mg).

LCMS (Method 1), Rt=0.82 min, ES⁺ m/z 698.3 [M+H]⁺

### Step 2

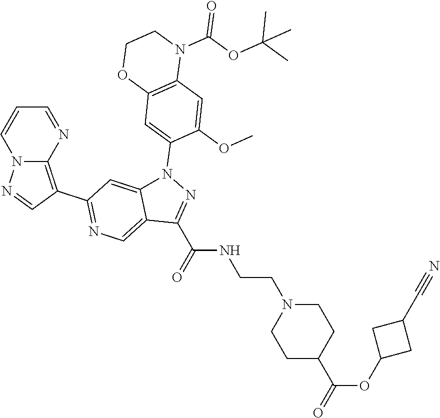 [SMILES: COc1cc2c(cc1-n1nc(C(=O)NCCN3CCC(C(=O)OC4CC(C#N)C4)CC3)c3cnc(-c4cnn5cccnc45)cc31)OCCN2C(=O)OC(C)(C)C]

### tert-butyl 7-(3-((2-(4-((3-cyanocyclobutoxy)carbonyl)piperidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate-(Example 47-intermediate 2)

To a mixture of Example 47-intermediate 1 (70.0 mg, 0.10 mmol), 2,4,6-trichlorobenzoyl chloride (39 μL, 0.25 mmol) and 3-hydroxycyclobutanecarbonitrile (21.4 μL, 0.25 mmol) in dry DMF (1 mL), TEA (70 μL, 0.50 mmol) and DMAP (3 mg, 25 μmol) were added and RM stirred at RT overnight. RM was diluted with EtOAc (15 mL) and washed with sat. aq. NaHCO₃ (3×10 mL), sat. aq. NH₄Cl (15 mL) and sat. aq. NaCl (10 mL). Organic layer was dried over Na₂SO₄ and concentrated in vacuo. The residue was purified by flash chromatography on Si cartridge by eluting with 0-50% DCM/MeOH (95:5) in DCM to afford the title product (47 mg).

LCMS (Method 2): Rt=1.21 min, ES⁺ m/z 777.4 [M+H]⁺

### Step 3

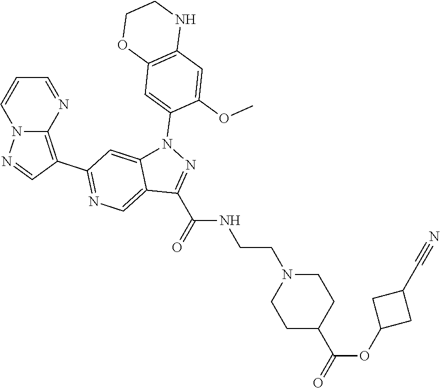 [SMILES: COc1cc2c(cc1-n1nc(C(=O)NCCN3CCC(C(=O)OC4CC(C#N)C4)CC3)c3cnc(-c4cnn5cccnc45)cc31)OCCN2]

### 3-cyanocyclobutyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate (Example 47)

An ice cold mixture of Example 47-intermediate 2 (45.0 mg, 57.9 μmol) in dry DCM (1 ml) was treated with TFA (222 μL, 2.90 mmol). RM was stirred for 2 h at RT, then diluted with DCM, cooled to 0° C., neutralized with aq. sat. NaHCO₃ and extracted with DCM (3×10 mL). Combined organic layers were passed through phase separator and solvent removed in vacuo. The residue was purified by flash chromatography on Si cartridge by eluting with 0-50% DCM/MeOH (9:1) in DCM to afford the title product (31 mg).

LCMS (Method 3): Rt=4.26 min, ES⁺ m/z 677.5 [M+H]⁺

¹H-NMR (600 MHz, DMSO-d₆) δ: 9.46 (d, J=1.1 Hz, 1H), 9.24 (dd, J=7.0, 1.7 Hz, 1H), 8.91 (s, 1H), 8.74 (dd, J=4.1, 1.7 Hz, 1H), 8.45 (t, J=5.9 Hz, 1H), 8.24 (d, J=1.1 Hz, 1H), 7.17 (dd, J=7.0, 4.0 Hz, 1H), 6.89 (s, 1H), 6.49 (s, 1H), 6.37-6.40 (m, 1H), 4.87 (quin, J=7.3 Hz, 1H), 4.14 (t, J=4.4 Hz, 2H), 3.63 (s, 3H), 3.44 (q, J=6.5 Hz, 2H), 3.37-3.41 (m, 2H), 3.03-3.12 (m, 1H), 2.82-2.90 (m, 2H), 2.70-2.78 (m, 2H), 2.51-2.53 (m, 2H), 2.26-2.34 (m, 3H), 2.01-2.08 (m, 2H), 1.77-1.85 (m, 2H), 1.51-1.61 (m, 2H).

### Example-48-Intermediate 1 and Example 49-Intermediate 1

The following intermediates were prepared in a similar manner to example 47-intermediate 1 from the indicated starting materials.

| Intermediate | Structure | Starting materials | LCMS |
|---|---|---|---|
| Example 48- intermediate 1 | 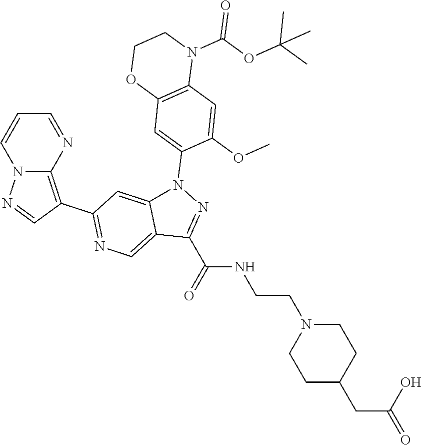 [SMILES: COc1cc2c(cc1-n1nc(C(=O)NCCN3CCC(CC(=O)O)CC3)c3cnc(-c4cnn5cccnc45)cc31)OCCN2C(=O)OC(C)(C)C] | Intermediate step 1 of example 35: tert-butyl 7- (3-((2-(4-(2-ethoxy-2- oxoethyl)piperidin-1- yl)ethyl)carbamoyl)-6- (pyrazolo[1,5-a]pyrimidin- 3-yl)-1H-pyrazolo[4,3- c]pyridin-1-yl)-6- methoxy-2,3-dihydro-4H- benzo[b][1,4]oxazine-4- carboxylate | (Method 1): Rt = 0.84 min, ES⁺ m/z 712.4 [M + H]⁺ |
| Example 49- intermediate 1 | 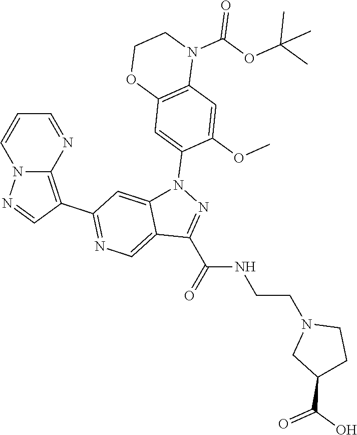 [SMILES: COc1cc2c(cc1-n1nc(C(=O)NCCN3CC[C@@H](C(=O)O)C3)c3cnc(-c4cnn5cccnc45)cc31)OCCN2C(=O)OC(C)(C)C] | Intermediate step 1 of example 29: tert-butyl (R)- 6-methoxy-7-(3-((2-(3- (methoxycarbonyl)pyrrolidin- 1-yl)ethyl)carbamoyl)- 6-(pyrazolo[1,5- a]pyrimidin-3-yl)-1H- pyrazolo[4,3-c]pyridin-1- yl)-2,3-dihydro-4H- benzo[b][1,4]oxazine-4- carboxylate | (Method 1): Rt = 0.82 min, ES⁺ m/z 684.4 [M + H]⁺ |

### Example 48 to 53

The following Examples were prepared with a two-step process (step 1 and step 2) similarly to Example 47-step 2 and step 3 respectively from the indicated starting materials. It is additionally stated if base, solvent, temperature or coupling reagent were varied.

| | | Step 1 | | | |
|---|---|---|---|---|---|
| | | Starting | Name of intermediate Step 1/ | Step 2 | |
| Example | Structure/name | material | LCMS | ¹H-NMR | LCMS |
| Example 48 | 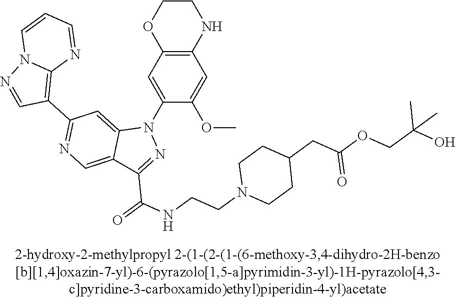 [SMILES: COc1cc2c(cc1-n1nc(C(=O)NCCN3CCC(CC(=O)OCC(C)(C)O)CC3)c3cnc(-c4cnn5cccnc45)cc31)OCCN2] | Example 48- intermediate 1 and 2-meth- ylpropane- 1,2-diol | tert-Butyl 7-(3-((2-(4-(2-(2- hydroxy-2-methylpropoxy)-2- oxoethyl)piperidin-1-yl)ethyl)- carbamoyl)-6-(pyrazolo[1,5-a] pyrimidin-3-yl)-1H-pyrazolo [4,3-c]pyridin-1-yl)-6-methoxy- 2,3-dihydro-4H-benzo[b][1,4] oxazine-4-carboxylate (Method 2): Rt = 1.24 min, ES⁺ m/z 784.5 [M + H]⁺ | (500 MHz, DMSO-d₆₎ δ: 9.45 (d, J = 1,1 Hz, 1H), 9.23 (dd, J = 6.9, 1.6 HZ, 1H), 8.91 (s, 1H), 8.73 (dd, J = 4.1, 1.6 Hz, 1H), 8.41 (t, J = 5.7 Hz, 1H), 8.24 (d, J = 1.1 Hz, 1H), 7.16 (dd, J = 7.0, 4.1 Hz, 1H), 6.88 (s, 1H), 6.49 (s, 1H), 6.38 (bs, 1H), 4.14 (t, J = 4.4 Hz, 2H), 3.79 (s, 2H), 3.63 (s, 3H), 3.43 (q, J = 6.4 Hz, 2H), 3.37-3.40 (m, 2H), 2.87- 2.90 (m, 2H), 2.48 (m, 2H, overlapped with DMSO), 2.25 (d, J = 6.9 Hz, 2H), 1.92-1.97 (m, 2H), 1.66-1.72 (m, 1H), 1.61-1.65 (m, 2H), 1.16-1.23 (m, 2H), 1.08 (s, 6H) | (Method 3): Rt = 4.06 min, ES⁺ m/z 684.3 [M + H]⁺ |
| Example 49 | 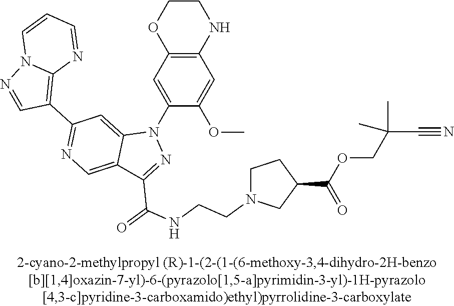 [SMILES: COc1cc2c(cc1-n1nc(C(=O)NCCN3CC[C@@H](C(=O)OCC(C)(C)C#N)C3)c3cnc(-c4cnn5cccnc45)cc31)OCCN2] | Example 49- intermediate 1 and 3- hydroxy- 2,2-dimeth- yl-propane- nitrile | tert-butyl (R)-7-(3-((2-(3-((2- cyano-2-methylpropoxy)carbon- yl)pyrrolidin-1-yl)ethyl)-carbam- oyl)-6-(pyrazolo[1,5-a]pyrimidin- 3-yl)-1H-pyrazolo[4,3-c]pyridin- 1-yl)-6-methoxy-2,3-dihydro-4H- benzo[b][1,4]oxazine-4-carbox- ylate (Method 2): Rt = 1-23 min, ES⁺ m/z 765.4 [M + H]⁺ | (600 MHz, DMSO-d₆) δ: 9.46 (d, J = 1.1 Hz, 1H), 9.24 (dd, J = 7.0, 1.8 Hz, 1H), 8.92 (s, 1H), 8.74 (dd, J = 4.1, 1.7 Hz, 1H), 8.47 (t, J = 5.9 Hz,1H), 8.24 (d, J = 1.3 Hz, 1H), 7.17 (dd, J = 7.0, 4.0 Hz, 1H), 6.89 (s, 1H), 6.49 (s, 1H), 6.37-6.40 (m, 1H), 4.14 (t, J = 4.3 Hz, 2H), 4.08 (s, 2H), 3.63 (s, 3H), 3.44 (q, J = 6.6 Hz, 2H), 3.37-3.41 (m, 2H), 3.07- 3.16 (m, 1H), 2.88 (t, J = 8.8 Hz, 1H), 2.69- 2.73 (m, 1H), 2.60-2.66 (m, 3H), 2.54-2.59 (m, 1H), 1.94-2.09 (m, 2H), 1.32 (s, 6H). | (Method 3): Rt = 4.38 min, ES⁺ m/z 665.6 [M + H]⁺ |
| Example 50 | 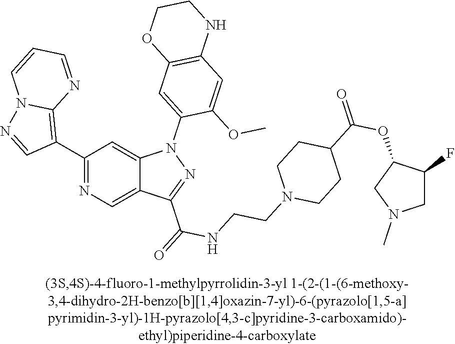 [SMILES: COc1cc2c(cc1-n1nc(C(=O)NCCN3CCC(C(=O)O[C@H]4CN(C)C[C@@H]4F)CC3)c3cnc(-c4cnn5cccnc45)cc31)OCCN2] | Example 47- intermediate 1 and Intermediate 15 | tert-butyl 7-(3-((2-(4-((((3S,4S)- 4-fluoro-1-methylpyrrolidin-3- yl)oxy)carbonyl)piperidin-1-yl)- ethyl)carbamoyl)-6-(pyrazolo [1,5-a]pyrimidin-3-yl)-1H-pyr- azolo[4,3-c]pyridin-1-yl)-6- methoxy-2,3-dihydro-4H-benzo [b][1,4]oxazine-4-carboxylate (Method 2): Rt = 0.86 min, ES⁺ m/z 799.4 [M + H]⁺ | (500 MHz, DMSO-d₆) δ: 9.46 (s, 1H), 9.23 (dd, J = 7.0, 1.5 Hz, 1H), 8.91 (s, 1H), 8.73 (dd, J = 4.1, 1.7 Hz, 1H), 8.44 (t, J = 5.8 Hz, 1H), 8.24 (s, 1H), 7.17 (dd, J = 6.9, 4.1 Hz, 1H), 6.88 (s, 1H), 6.49 (s, 1H), 6.38 (br s, 1H), 5.03-5.10 (m, 1H), 4.93-5.03 (m, 1H), 4.14 (br t, J = 4.3 Hz, 2H), 3.63 (s, 3H), 3.44 (q, J = 6.4 Hz, 2H), 3.39 (m, 2H), 3.07 (dd, J = 10.4, 6.7 Hz, 1H), 2.86 (m, 2H), 2.73- 2.80 (m, 1H), 2.66-2.73 (m, 1H), 2.47-2.54 (m, 2H), 2.30-2.36 (m, 1H), 2.26-2.29 (m, 1H), 2.24 (s, 3H), 2.05 (br t, J = 10.4 Hz, 2H), 1.75-1.87 (m, 2H), 1.48-1.65 (m, 2H). | (Method 3): Rt = 3.43 min, ES⁺ m/z 699.3 [M + H]⁺ |
| Example 51 | 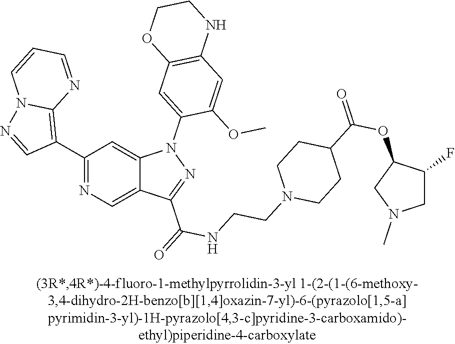 | Example 47- intermediate 1 and (3R*,4R*)- 4-fluoro-1- methylpyr- rolidin-3-ol | tert-butyl 7-(3-((2-(4-((((3R*, 4R*)-4-fluoro-1-methylpyrrolidin- 3-yl)oxy)carbonyl)piperidin-1- yl)ethyl)carbamoyl)-6-(pyrazolo [1,5-a]pyrimidin-3-yl)-1H-pyr- azolo[4,3-c]pyridin-1-yl)-6-meth- oxy-2,3-dihydro-4H-benzo [b][1,4]oxazine-4-carboxylate (Method 2): Rt = 0.86 min, ES⁺ m/z 799.4 [M + H]⁺ | (500 MHz, DMSO-d₆) δ: 9.45 (s, 1H), 9.23 (dd, J = 6.9, 1.0 HZ, 1H), 8.91 (s, 1H), 8.73 (dd, J = 3.9, 1.1 Hz, 1H), 8.41 (t, J = 5.7 Hz, 1H), 8.24 (s, 1H), 7.16 (dd, J = 6.8, 4.0 Hz, 1H), 6.88 (s, 1H), 6.49 (s, 1H), 6.38 (bs, 1H), 4.95-5.07 (m, 2H), 4.13-4.17 (t, J = 3.5 Hz, 2H), 3.62 (s, 3H), 3.43 (q, J = 6.0 Hz, 2H), 3.39 (m, 2H), 3.07 (dd, J = 10.3, 6.8 Hz, 1H), 2.83-2.89 (m, 2H), 2.67-2.78 (m, 2H), 2.49 (m, 2H, overlapped with DMSO signal), 2.30-2.36 (m, 1H), 2.27 (dd, J = 10.4, 4.7 Hz, 1H), 2.24 (s, 3H), 2.05 (t, J = 10.5 Hz, 2H), 1.78-1.82 (m, 2H), 1.52-1.61 (m, 2H). | (Method 3): Rt = 3.51 min, ES⁺ m/z 699.3 [M + H]⁺ |
| Example 52 | 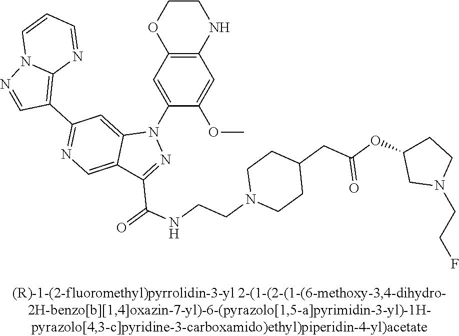 [SMILES: COc1cc2c(cc1-n1nc(C(=O)NCCN3CCC(CC(=O)O[C@@H]4CCN(CCF)C4)CC3)c3cnc(-c4cnn5cccnc45)cc31)OCCN2] | Example 48- intermediate 1 and Intermediate 16 | tert-Butyl (R)-7-(3-((2-(4-(2-((1- (2-fluoroeth-yl)pyrrolidin-3-yl)- oxy)-2-oxoethyl)piperidin-1-yl)- ethyl)carbamoyl)-6-(pyrazolo [1,5-a]pyrimidin-3-yl)-1H-pyr- azolo[4,3-c]pyridin-1-yl)-6- methoxy-2,3-dihydro-4H-benzo [b][1,4]oxazine-4-carboxylate (Method 1): Rt = 0.74 min, ES⁺ m/z 827.4 [M + H]⁺ | (500 MHz, DMSO-d₆) δ: 9.46 (d, J = 1.2 Hz, 1H), 9.24 (dd, J = 7.0, 1.5 Hz, 1H), 8.91 (s, 1H), 8.74 (dd, J = 4.0, 1.5 Hz, 1H), 8.42 (t, J = 5.8 Hz, 1H), 8.24 (d, J = 0.9 Hz, 1H), 7.17 (dd, J = 6.9, 4.1 Hz, 1H), 6.88 (s, 1H), 6.49 (s, 1H), 6.37-6.40 (m, 1H), 5.02-5.08 (m, 1H), 4.49 (dt, J = 47.6, 4.9 Hz, 2H), 4.14 (br t, J = 3.8 Hz, 2H), 3.63 (s, 3H), 3.40-3.46 (m, 2H), 3.35-3.40 (m, 2H), 2.89 (br d, J = 11.0 Hz, 2H), 2.57-2.77 (m, 5H), 2.45-2.50 (m, 2H), 2.34-2.41 (m, 1H), 2.20 (br d, J = 6.7 Hz, 2H), 2.10-2.18 (m, 1H), 1.95 (br t, J = 11.1 Hz, 2H), 1.56-1.70 (m, 4H), 1.14-1.25 (m, 2H). | (Method 3): Rt = 3.50 min, ES⁺ m/z 727.3 [M + H]⁺ |
| Example 53 | 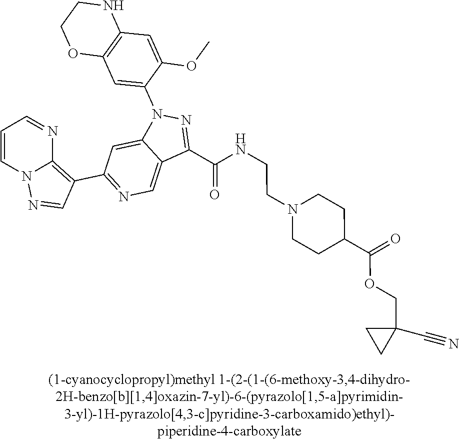 [SMILES: COc1cc2c(cc1-n1nc(C(=O)NCCN3CCC(C(=O)OCC4(C#N)CC4)CC3)c3cnc(-c4cnn5cccnc45)cc31)OCCN2] | Example 47- intermediate l and 1- (hydroxy- methyl)cyclo- propane- carbonitrile | tert-butyl 7-(3-((2-(4-(((1-cyano- cyclopropyl)methoxy)carbonyl)- piperidin-1-yl)ethyl)carbamoyl)-6- (pyrazolo[1,5-a]pyrimidin-3-yl)- 1H-pyrazolo[4,3-c]pyridin-1-yl)- 6-methoxy-2,3-dihydro-4H-benzo [b][1,4]oxazine-4-carboxylate (Method 1): Rt = 0.92 min, ES⁺ m/z 777.4 [M + H]⁺ | (500 MHz, DMSO-d₆) δ: 9.46 (d, J = 0.9 Hz, 1H), 9.23 (dd, J = 7.0, 1.5 Hz, 1H), 8.91 (s, 1H), 8.73 (dd, J = 4.0, 1.8 Hz, 1H), 8.46 (t, J = 5.8 Hz, 1H), 8.24 (d, J = 0.9 Hz, 1H), 7.16-7.18 (m, 1H), 6.89 (s, 1H), 6.49 (s, 1H), 6.38 (s, 1H), 4.14 (t, J = 4.3 Hz, 2H), 4.07 (s, 2H), 3.63 (s, 3H), 3.44 (q, J = 6.6 Hz, 2H), 3.39 (m, 2H), 2.87 (m, 2H), 2.51- 2.54 (m, 2H), 2.37-2.44 (m, 1H), 2.05-2.12 (m, 2H), 1.84 (m, 2H), 1.57-1.65 (m, 2H), 1.29-1.32 (m, 2H), 1.09-1.14 (m, 2H). | (Method 3): Rt = 4.24 min, ES⁺ m/z 677.3 [M + H]⁺ |

### Example 54

### Step 1

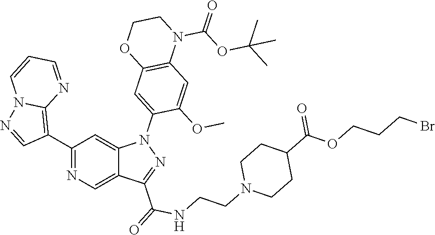 [SMILES: COc1cc2c(cc1-n1nc(C(=O)NCCN3CCC(C(=O)OCCCBr)CC3)c3cnc(-c4cnn5cccnc45)cc31)OCCN2C(=O)OC(C)(C)C]

### tert-Butyl 7-(3-((2-(4-((3-bromopropoxy)carbonyl)piperidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 54-intermediate 1)

Title product was prepared on a similar manner to Example 47-intermediate 2 starting from Example 47-intermediate 1 and 3-bromopropan-1-ol.

LCMS (Method 2): Rt=1.33 min, ES⁺ m/z 818.3/820.2 [M+H]⁺

### Step 2

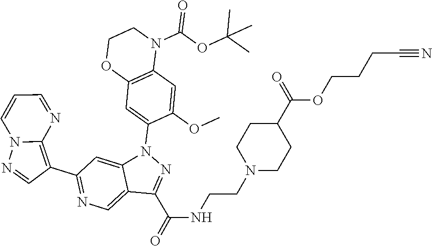 [SMILES: COc1cc2c(cc1-n1nc(C(=O)NCCN3CCC(C(=O)OCCCC#N)CC3)c3cnc(-c4cnn5cccnc45)cc31)OCCN2C(=O)OC(C)(C)C]

### tert-Butyl 7-(3-((2-(4-((3-cyanopropoxy)carbonyl)piperidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 54-intermediate 2)

A mixture of Example 54-intermediate 1 (32.0 mg, 0.04 mmol) and NaCN (10.0 mg, 0.20 mmol) in DMF (1.0 mL) was stirred at 80° C. for 1.5 h. RM was diluted with EtOAc (15 mL), washed with sat. aq. NaHCO₃ (3×10 mL) and sat. aq. NaCl (10 mL). Organic layer was dried over Na₂SO₄ and concentrated in vacuo. The residue was purified by flash chromatography on Si cartridge by eluting with 0-70% DCM/MeOH (9:1) in DCM to afford the title product (28 mg).

LCMS (Method 2): Rt=1.18 min, ES⁺ m/z 765.4 [M+H]⁺

### Step 3

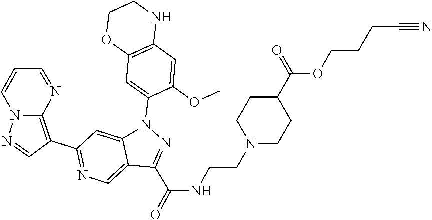 [SMILES: COc1cc2c(cc1-n1nc(C(=O)NCCN3CCC(C(=O)OCCCC#N)CC3)c3cnc(-c4cnn5cccnc45)cc31)OCCN2]

### 3-Cyanopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate (Example 54)

Title compound was prepared on a similar manner to step 3 of Example 47 starting from Example 54-intermediate 2.

LCMS (Method 3): Rt=4.12 min, ES⁺ m/z 665.4 [M+H]⁺

¹H-NMR (500, DMSO-d₆) δ: 9.45 (d, J=1.1 Hz, 1H), 9.23 (dd, J=7.0, 1.5 Hz, 1H), 8.91 (s, 1H), 8.73 (dd, J=4.3, 1.5 Hz, 1H), 8.45 (t, J=5.8 Hz, 1H), 8.24 (d, J=1.2 Hz, 1H), 7.17 (dd, J=7.0, 4.0 Hz, 1H), 6.89 (s, 1H), 6.49 (s, 1H), 6.35-6.41 (m, 1H), 4.14 (t, J=4.3 Hz, 2H), 4.08 (t, J=6.1 Hz, 2H), 3.63 (s, 3H), 3.44 (q, J=6.9 Hz, 2H), 3.36-3.41 (m, 2H), 2.80-2.93 (m, 2H), 2.56 (t, J=7.2 Hz, 2H), 2.49-2.52 (m, 2H), 2.28-2.35 (m, 1H), 2.04 (br t, J=10.5 Hz, 2H), 1.88 (quin, J=6.6 Hz, 2H), 1.78-1.85 (m, 2H), 1.51-1.64 (m, 2H).

### Example 55

### Step 1

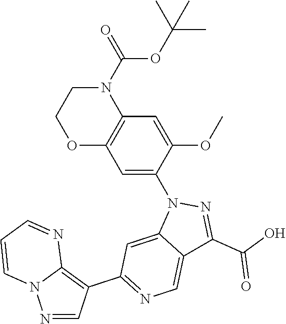 [SMILES: COc1cc2c(cc1-n1nc(C(=O)O)c3cnc(-c4cnn5cccnc45)cc31)OCCN2C(=O)OC(C)(C)C]

### 1-(4-(tert-Butoxycarbonyl)-6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylic acid (Example 55-Intermediate 1)

To a previously degassed mixture of aqueous potassium phosphate (1.0 M, 2.17 mL, 2.2 mmol) and THF (5 mL), intermediate 7 (500 mg, 1.1 mmol), 3-(4,4,5,5-tetramethyl-1,3,2-dioxaborolan-2-yl)pyrazolo[1,5-a]pyrimidine (372 mg, 1.5 mmol) and XPhos-PdG3 (46 mg, 54 μmol) were added and RM stirred at 70° C. for 1.5 h. After cooling to RT, RM was diluted with water (15 mL) and basified to pH 10. RM was washed with EtOAc (2×20 mL) and aqueous layer filtered through a pad of diatomaceous earth and washed with water (100 mL). A mixture of MEK (methylethyl ketone) and i-BMK (isobutylmethyl ketone) (5:1-150 mL) was added and pH adjusted to 4.5. Organic layer was separated, dried over Na₂SO₄, filtered, and evaporated to dryness to give the title product (560 mg) that was used in the next synthetic steps without further purification.

LCMS (Method 1): Rt=1.18 min, ES⁺ m/z 544.4 [M+H]⁺

### Step 2

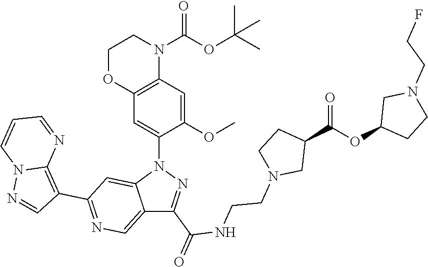 [SMILES: COc1cc2c(cc1-n1nc(C(=O)NCCN3CC[C@@H](C(=O)O[C@@H]4CCN(CCF)C4)C3)c3cnc(-c4cnn5cccnc45)cc31)OCCN2C(=O)OC(C)(C)C]

### tert-butyl 7-(3-((2-((R)-3-((((R)-1-(2-fluoroethyl)pyrrolidin-3-yl)oxy)carbonyl) pyrrolidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 55-Intermediate 2)

A solution of Intermediate 17 (140 mg, 0.34 mmol) in conc. HCl (1.5 mL) was stirred overnight at RT. Solvent was removed in vacuo, residue dried in vacuum oven at 40° C. for 72h. To the residue taken in dry DMF (2 mL), Example 55-Intermediate 1 (171 mg, 0.32 mmol), DIPEA (507 μL, 2.96 mmol) and HATU (169 mg, 0.44 mmol) were added and RM stirred at RT for 1h. A second equivalent of HATU and were added and RM stirred at RT for further 1h. RM was quenched with sat. aq. NH₄Cl and the formed precipitate collected by filtration, washed several times with water. The obtained solid was purified by flash chromatography on Si cartridge by eluting with 0-40% DCM/MeOH/NH₄OH (90:9:1.5) in DCM to afford the title product (10 mg).

LCMS (Method 2): Rt=1.18 min, ES⁺ m/z 799.4 [M+H]⁺

### Step 2

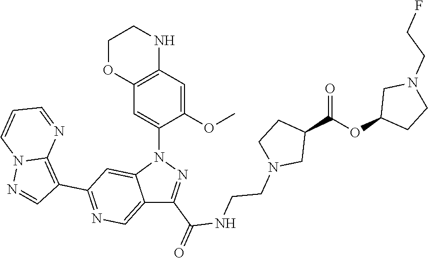 [SMILES: COc1cc2c(cc1-n1nc(C(=O)NCCN3CC[C@@H](C(=O)O[C@@H]4CCN(CCF)C4)C3)c3cnc(-c4cnn5cccnc45)cc31)OCCN2]

### (R)-1-(2-fluoroethyl)pyrrolidin-3-yl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate (Example 55)

Title compound was prepared on a similar manner to step 3 of Example 47 starting from Example 55-Intermediate 2.

LCMS (Method 3): Rt=3.44 min, ES⁺ m/z 699.3 [M+H]⁺

¹H-NMR (500 MHz, DMSO-d₆) δ: 9.46 (d, J=1.2 Hz, 1H), 9.23 (dd, J=7.0, 1.5 Hz, 1H), 8.91 (s, 1H), 8.73 (dd, J=4.1, 1.7 Hz, 1H), 8.45 (t, J=6.0 Hz, 1H), 8.24 (d, J=0.9 Hz, 1H), 7.17 (dd, J=7.0, 4.0 Hz, 1H), 6.88 (s, 1H), 6.49 (s, 1H), 6.37-6.40 (m, 1H), 5.02-5.08 (m, 1H), 4.48 (dt, J=48.0, 4.7 Hz, 2H), 4.14 (t, J=4.3 Hz, 2H), 3.63 (s, 3H), 3.43 (q, J=6.4 Hz, 2H), 3.37-3.41 (m, 2H), 2.94-3.03 (m, 1H), 2.83 (t, J=8.7 Hz, 1H), 2.56-2.75 (m, 9H), 2.51-2.55 (m, 1H), 2.33-2.40 (m, 1H), 2.08-2.20 (m, 1H), 1.88-2.03 (m, 2H), 1.61-1.71 (m, 1H).

### Example 56 to 57

The following Examples were prepared with a two step process similarly to Example 9 from the indicated starting materials.

| | | Step 1 | | | |
|---|---|---|---|---|---|
| | | Starting | Name of intermediate Step 1/ | Step 2 | |
| Example | Structure/name | material | LCMS | ¹H-NMR | LCMS |
| Example 56 | 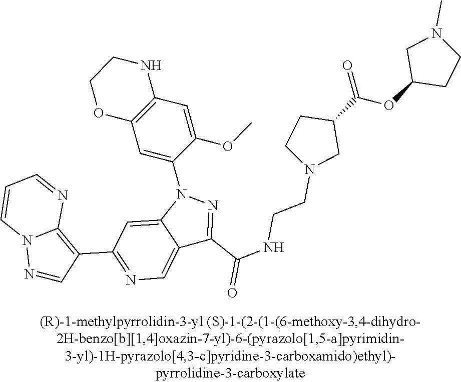 [SMILES: COc1cc2c(cc1-n1nc(C(=O)NCCN3CC[C@H](C(=O)O[C@@H]4CCN(C)C4)C3)c3cnc(-c4cnn5cccnc45)cc31)OCCN2] | Int 20a | tert-butyl 6-methoxy-7-(3-((2-((S)-3- (((R)-1-methylpyrrolidin-3-yl)oxy) carbonyl)pyrrolidin-1-yl)ethyl)- carbamoyl)-6-(pyrazolo[1,5-a] pyrimidin-3-yl)-1H-pyrazolo[4,3- c]pyridin-1-yl)-2,3-dihydro-4H- benzo[b][1,4]oxazine-4-carboxylate (Method 1): Rt = 0.79 min, ES⁺ m/z 767.5 [M + H]⁺ | (500 MHz, DMSO-d₆) δ: 9.46 (d, J = 1.2 Hz, 1H), 9.24 (dd, J = 7.0, 1.5 Hz, 1H), 8.91 (s, 1H), 8.74 (dd, J = 4.1, 1.7 Hz, 1H), 8.45 (t, J = 5.8 Hz, 1H), 8.24 (d, J = 1.2 Hz, 1H), 7.17 (m, 1H), 6.88 (s, 1H), 6.49 (s, 1H), 6.39 (s, 1H), 5.04 (m, 1H), 4.14 (t, J = 4.4 Hz, 2H), 3.63 (s, 3H), 3.44 (q, J = 6.5 Hz, 2H), 3.39 (m, 2H), 2.98 (m, 1H), 2.83 (t, J = 8.7 Hz, 1H), 2.61 (m, 6H), 2.53 (m, overlapping with DMSO, 2H), 2.22 (m, 1H), 2.21 (s, 3H), 2.13 (m, 1H), 1.95 (m, 2H), 1.65 (m, 1 H). | (Method 3): Rt = 3.41 min, ES⁺ m/z 667.3 [M + H]⁺ |
| Example 57 | 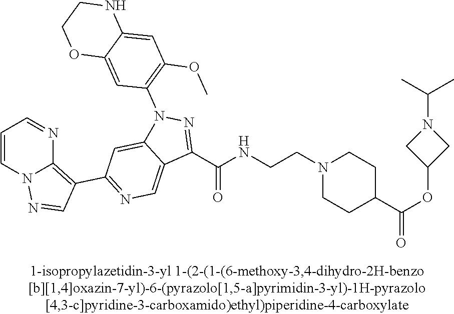 [SMILES: COc1cc2c(cc1-n1nc(C(=O)NCCN3CCC(C(=O)OC4CN(C(C)C)C4)CC3)c3cnc(-c4cnn5cccnc45)cc31)OCCN2] | Int. 20b | tert-butyl 7-(3-((2-(4-(((1-isopropyl- azetidin-3-yl)oxy)carbonyl)piper- idin-1-yl)ethyl)carbamoyl)-6-(pyr- azolo[1,5-a]pyrimidin-3-yl)-1H- pyrazolo[4,3-c]pyridin-1-yl)-6- methoxy-2,3-dihydro-4H-benzo [b][1,4]oxazine-4-carboxylate (Method 1): Rt = 0.82 min, ES⁺ m/z 795.5 [M + H]⁺ | (500 MHz, DMSO-d₆) δ: 9.46 (d, J = 1.0 Hz, 1H), 9.23 (dd, J = 7.0, 1.6 Hz, 1H), 8.91 (s, 1H), 8.73 (dd, J = 4.2, 1.6 Hz, 1H), 8.45 (t, J = 5.0 Hz, 1H), 8.24 (s, 1H), 7.17 (dd, J = 7.2, 4.1 Hz, 1H), 6.88 (s, 1H), 6.49 (s, 1H), 6.38 (bs, 1H), 4.85 (quint, J = 6.0 Hz, 1H), 4.14 (t, J = 4.1 Hz, 2H), 3.63 (s, 3H), 3.57-3.50 (m, 2H), 3.47-3.41 (m, 2H), 3.41-3.37 (m, 2H), 2.92-2.81 (m, 4H), 2.54-2.50 (m, 2H, overlapped with DMSO), 2.34-2.26 (m, 2H), 2.11-2.01 (m, 2H), 1.83-1.77 (m, 2H), 1.61-1.51 (m, 2H), 0.84 (d, J = 6.1 Hz, 6H). | (Method 3): Rt = 3.54 min, ES⁺ m/z 695.3 [M + H]⁺ |

### Example 58

### Step 1

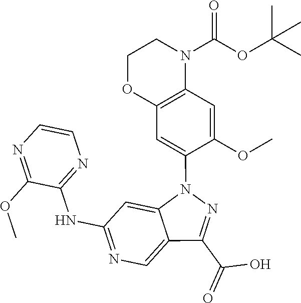 [SMILES: COc1cc2c(cc1-n1nc(C(=O)O)c3cnc(Nc4nccnc4OC)cc31)OCCN2C(=O)OC(C)(C)C]

### 1-(4-(tert-Butoxycarbonyl)-6-methoxy-3,4-dihydro-2H-benzo[b][1,4]oxazin-7-yl)-6-((3-methoxypyrazin-2-yl)amino)-1H-pyrazolo[4,3-c]pyridine-3-carboxylic acid (Example 58-Intermediate 1)

To a Shlenk tube charged with Intermediate 7 (100 mg, 0.22 mmol), 3-methoxypyrazin-2-amine (41 mg, 0.33 mmol) and RuPhos-Pd-G3 (9.1 mg, 11 μmol), under nitrogen dioxane (3 mL) and LiHMDS (1.3 M in THF, 826 μL, 1.1 mmol) were added. RM was heated at 100° C. for 20 h. After cooling to RT, RM was diluted with water (30 mL) and extracted with EtOAc (2×15 mL). Aqueous layer was acidified with 1N HCl (20 mL) and extracted with MEK (methylethyl ketone) (3×15 mL). Combined organic layers were dried over Na₂SO₄, filtered and solvent removed in vacuo. The residue was suspended in water (30 mL) and extracted with EtOAc (2×20 mL). Combined organic layers were dried over Na₂SO₄, filtered and solvent removed in vacuo to afford the title compound (84 mg) that was used in the next synthetic steps without further purifications.

LCMS (Method 2): Rt=0.87 min, ES⁺ m/z 550.2 [M+H]⁺

### Step 2

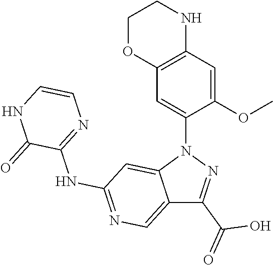 [SMILES: COc1cc2c(cc1-n1nc(C(=O)O)c3cnc(Nc4ncc[nH]c4=O)cc31)OCCN2]

### 1-(6-Methoxy-3,4-dihydro-2H-benzo[b][1,4]oxazin-7-yl)-6-((3-oxo-3,4-dihydropyrazin-2-yl)amino)-1H-pyrazolo[4,3-c]pyridine-3-carboxylic acid (Example 58-Intermediate 2)

A mixture of Example 58-intermediate 1 (84 mg, 0.15), sodium iodide (92 mg, 0.61 mmol) and TMS-Cl (78 μL, 0.61 mmol) in acetonitrile (2.0 mL) was stirred at 85° C. for 3 h. RM was cooled to RT and quenched with water (50 μL). Toluene (5 mL) was added, and solvents removed in vacuo to afford the title compound (290 mg) that was used in the next synthetic step without further purifications.

LCMS (Method 2): Rt=0.49 min, ES⁺ m/z 436.2 [M+H]⁺

### Step 3

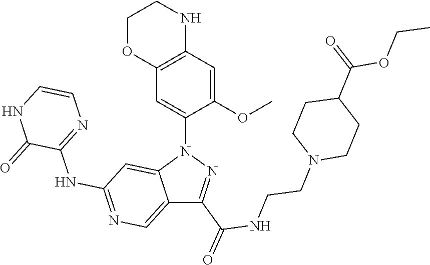 [SMILES: CCOC(=O)C1CCN(CCNC(=O)c2nn(-c3cc4c(cc3OC)NCCO4)c3cc(Nc4ncc[nH]c4=O)ncc23)CC1]

### Ethyl 1-(2-(1-(6-methoxy-3,4-dihydro-2H-benzo[b][1,4]oxazin-7-yl)-6-((3-oxo-3,4-dihydropyrazin-2-yl)amino)-1H-pyrazolo[4,3-c]pyridine-3-carboxamido)ethyl)piperidine-4-carboxylate (Example 58)

Title compound was prepared on a similar manner to intermediate 11a starting from Example 58-Intermediate 2 and Intermediate 1.

LCMS (Method 3): Rt=4.10 min, ES⁺ m/z 618.3 [M+H]⁺

¹H-NMR (500 MHz, DMSO-d₆) δ: 9.15 (d, J=0.5 Hz, 1H), 8.81 (s, 1H), 8.39 (t, J=5.3 Hz, 1H), 8.14 (d, J=0.7 Hz, 1H), 6.94 (d, J=4.4 Hz, 1H), 6.89 (d, J=4.4 Hz, 1H), 6.83 (s, 1H), 6.46 (s, 1H), 6.35 (bs, 1H), 4.12 (d, J=3.9 Hz, 2H), 4.05 (q, J=7.1 Hz, 2H), 3.63 (s, 3H), 3.41 (q, J=6.7 Hz, 2H), 3.30 (m, 2H, overlapped with HDO), 2.83-2.86 (m, 2H), 2.48 (t, J=6.7 Hz, 2H), 2.27 (tt, J=6.7 Hz, 1H), 2.00-2.05 (m, 2H), 1.76-1.80 (m, 2H), 1.51-1.58 (m, 2H), 1.16 (t, J=7.1 Hz, 3H)

### Example 59

### Step 1

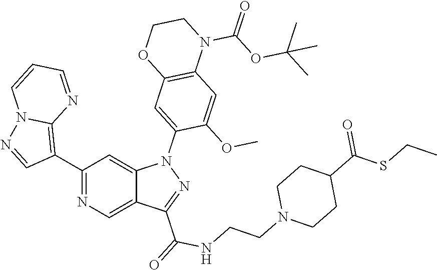 [SMILES: CCSC(=O)C1CCN(CCNC(=O)c2nn(-c3cc4c(cc3OC)N(C(=O)OC(C)(C)C)CCO4)c3cc(-c4cnn5cccnc45)ncc23)CC1]

### tert-Butyl 7-(3-((2-(4-((ethylthio)carbonyl)piperidin-1-yl)ethyl)carbamoyl)-6-(pyrazolo[1,5-a]pyrimidin-3-yl)-1H-pyrazolo[4,3-c]pyridin-1-yl)-6-methoxy-2,3-dihydro-4H-benzo[b][1,4]oxazine-4-carboxylate (Example 59-Intermediate 1)

Title product was prepared on a similar manner to step 2 of Example 47 starting from Example 47-intermediate 1 and ethanethiol.

LCMS (Method 1): Rt=1.00 min, ES⁺ m/z 742.4 [M+H]⁺

### Step 2

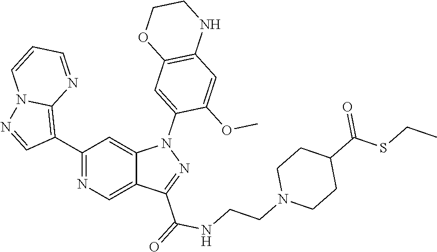 [SMILES: CCSC(=O)C1CCN(CCNC(=O)c2nn(-c3cc4c(cc3OC)NCCO4)c3cc(-c4cnn5cccnc45)ncc23)CC1]

### S-Ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carbothioate (Example 59)

Title compound was prepared similarly to step 3 of Example 47, starting from Example 59-intermediate 1.

LCMS (Method 4): Rt=4.47 ES⁺ m/z 642.2 [M+H]⁺

¹H-NMR (500 MHz, DMSO-d₆) δ: 9.46 (d, J=0.9 Hz, 1H), 9.23 (dd, J=7.0, 1.5 Hz, 1H), 8.91 (s, 1H), 8.73 (dd, J=4.1, 1.7 Hz, 1H), 8.45 (t, J=5.6 Hz, 1H), 8.24 (d, J=0.9 Hz, 1H), 7.17 (dd, J=7.0, 4.3 Hz, 1H), 6.89 (s, 1H), 6.49 (s, 1H), 6.38 (s, 1H), 4.14 (t, J=4.3 Hz, 2H), 3.63 (s, 3H), 3.43 (q, J=6.7 Hz, 2H), 3.39 (m, 2H), 2.92 (m, 2H), 2.82 (q, J=7.5 Hz, 2H), 2.51-2.54 (m, 2H), 2.45-2.49 (m, 1H), 2.03 (br t, J=10.7 Hz, 2H), 1.80 (m, 2H), 1.54-1.62 (m, 2H), 1.16 (t, J=7.3 Hz, 3H).

### PREPARATION OF COMPARATIVE EXAMPLES A1 to A12

### Example A1

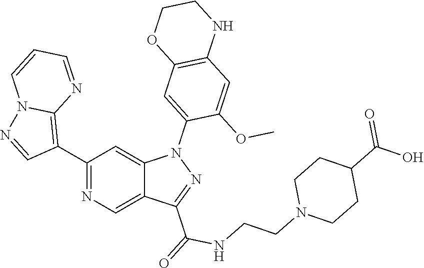 [SMILES: COc1cc2c(cc1-n1nc(C(=O)NCCN3CCC(C(=O)O)CC3)c3cnc(-c4cnn5cccnc45)cc31)OCCN2]

### 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylic acid (Example A1)

A solution of example 9 (46.1 mg, 0.06 mmol) in THF (6 mL) was treated with solution of LiOH·H₂O (6.60 mg, 0.18 mmol) in water (2 mL). RM was stirred at RT overnight. Organic solvent was removed in vacuo and pH was adjusted to 7 using aq. 1M HCl, the residue was then purified by HPLC-MDAP (Method 1) to afford the title compound (24.1 mg).

LCMS (Method 3): Rt=3.72 min, ES⁺ m/z 598.3

¹H-NMR (500 MHz, DMSO-d₆) δ: 9.45 (s, 1H), 9.22 (d, J=7.0 Hz, 1H), 8.90 (s, 1H), 8.73 (dd, J=4.3, 1.5 Hz, 1H), 8.46 (s, 1H), 8.24 (s, 1H), 7.16 (dd, J=7.0, 4.3 Hz, 1H), 6.88 (s, 1H), 6.49 (s, 1H), 6.36 (br s, 1H), 4.13 (br t, J=4.1 Hz, 2H), 3.63 (m, 5H, overlapped with HDO), 2.86 (br d, J=11.3 Hz, 2H), 2.54 (s, 1H), 2.53-2.51 (overlapped with DMSO-d6, 4H), 2.19 (br t, J=4.0 Hz, 1H), 2.04 (br t, J=10.5 Hz, 2H), 1.78 (br d, J=10.4 Hz, 2H), 1.59-1.49 (m, 2H).

### Examples A2 to A12

The following examples were prepared in a similar manner to example A1 from the indicated starting materia.

| | | Starting | | |
|---|---|---|---|---|
| Example | Structure/name | Material | ¹H-NMR | LCMS |
| A2 | 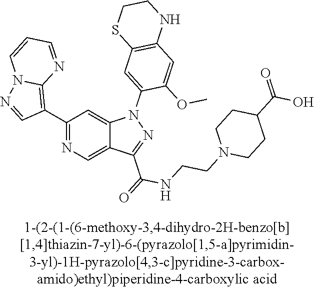 [SMILES: COc1cc2c(cc1-n1nc(C(=O)NCCN3CCC(C(=O)O)CC3)c3cnc(-c4cnn5cccnc45)cc31)SCCN2] | Example 1 | (500 MHz, DMSO-d₆) δ: 9.45 (d, J = 1.0 Hz, 1H), 9.23 (dd, J = 7.0, 1.6 Hz, 1H), 8.90 (s, 1H), 8.73 (dd, J = 3.9, 1.7 Hz, 1H), 8.41-8.53 (m, 1H), 8.23 (d, J = 1.0 Hz, 1H), 7.16 (dd, J = 7.0, 4.2 Hz, 1H), 7.08 (s, 1H), 6.58-6.74 (m, 1H), 6.44 (s, 1H), 3.64 (s, 3H), 3.54- 3.61 (m, 2H), 3.38-3.48 (m, 2H), 2.93-3.04 (m, 2H), 2.76- 2.90 (m, 2H), 2.35-2.57 (m, 2H), 2.09-2.22 (m, 1H), 1.94- 2.07 (m, 2H), 1.68-1.84 (m, 2H), 1.42-1.62 (m, 2H). | (Method 3): Rt = 4.00 min, ES⁺ m/z 614.2 [M + H]⁺ |
| A3 | 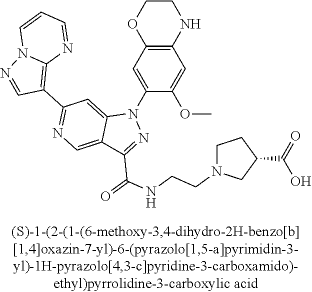 [SMILES: COc1cc2c(cc1-n1nc(C(=O)NCCN3CC[C@H](C(=O)O)C3)c3cnc(-c4cnn5cccnc45)cc31)OCCN2] | Example 44 | (600 MHz, DMSO-d₆) δ: 9.47 (d, J = 1.1 Hz, 1H), 9.24 (dd, J = 6.9, 1.5 Hz, 1H), 8.91 (s, 1H), 8.74 (dd, J = 4.2, 1.8 Hz, 1H), 8.50 (t, J = 5.6 Hz, 1H), 8.25 (d, J = 1.0 Hz, 1H), 7.18 (dd, J = 7.2, 4.1 Hz, 1H), 6.89 (s, 1H), 6.49 (s, 1H), 6.38-6.40 (m, 1H), 4.15 (t, J = 4.4 Hz, 2H), 3.63 (s, 3H), 3.43-3.50 (m, 4H overlapped with HDO); 2.83-2.95 (m, 2H), 2.60-2.66 (m, 4H), 2.50-2.54 (m, 1H, overlapped with DMSO), 1.91-1.98 (m, 2H). | (Method 3): Rt = 3.66 min, ES⁺ m/z 584.2 [M + H]⁺ |
| A4 | 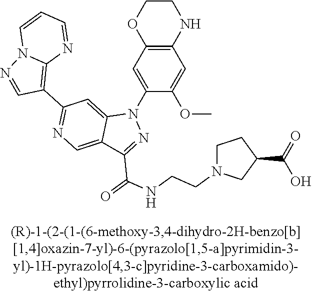 [SMILES: COc1cc2c(cc1-n1nc(C(=O)NCCN3CC[C@@H](C(=O)O)C3)c3cnc(-c4cnn5cccnc45)cc31)OCCN2] | Example 43 | (600 MHz, DMSO-d₆) δ: 9.47 (d, J = 1.1 Hz, 1H), 9.24 (dd, J = 6.9, 1.5 Hz, 1H), 8.91 (s, 1H), 8.74 (dd, J = 4.2, 1.8 Hz, 1H), 8.50 (t, J = 5.6 Hz, 1H), 8.25 (d, J = 1.0 Hz, 1H), 7.18 (dd, J = 7.2, 4.1 Hz, 1H), 6.89 (s, 1H), 6.49 (s, 1H), 6.38-6.40 (m, 1H), 4.15 (t, J = 4.4 Hz, 2H), 3.63 (s, 3H), 3.43-3.50 (m, 4H overlapped with HDO); 2.83-2.95 (m, 2H), 2.60-2.66 (m, 4H), 2.50-2.54 (m, 1H, overlapped with DMSO), 1.91-1.98 (m, 2H). | (Method 3): Rt = 3.65 min, ES⁺ m/z 584.2 [M + H]⁺ |
| A5 | 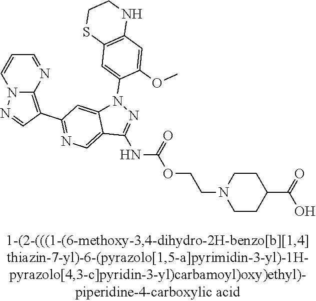 [SMILES: COc1cc2c(cc1-n1nc(NC(=O)OCCN3CCC(C(=O)O)CC3)c3cnc(-c4cnn5cccnc45)cc31)SCCN2] | Example 4 | (500 MHz, DMSO-d₆) δ: 12.13 (br s, 1H), 10.43 (br s, 1H), 9.25-9.28 (m, 1H), 9.22 (dd, J = 7.0, 1.5 Hz, 1H), 8.87 (s, 1H), 8.72 (dd, J = 4.1, 1.7 Hz, 1H), 8.08 (d, J = 0.9 Hz, 1H), 7.15 (dd, J = 7.0, 4.0 Hz, 1H), 6.93 (s, 1H), 6.55-6.59 (m, 1H), 6.42 (s, 1H), 4.27 (t, J = 5.6 Hz, 2H), 3.64 (s, 3H), 3.56-3.61 (m, 2H), 2.96-3.01 (m, 2H), 2.84-2.92 (m, 2H), 2.61 (t, J = 5.6 Hz, 2H), 2.15- 2.24 (m, 1H), 2.01-2.11 (m, 2H), 1.74-1.84 (m, 2H), 1.51- 1.65 (m, 2H). | (Method 3): Rt = 3.70 min, ES⁺ m/z 630.2 [M + H]⁺ |
| A6 | 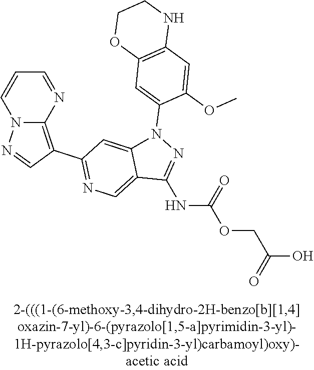 [SMILES: COc1cc2c(cc1-n1nc(NC(=O)OCC(=O)O)c3cnc(-c4cnn5cccnc45)cc31)OCCN2] | Example 39 | (300 MHz, DMSO-d₆) δ: 13.12 (br s, 1H), 10.67 (br s, 1H), 9.17-9.25 (m, 2H), 8.87 (s, 1H), 8.71 (dd, J = 4.0, 1.6 Hz, 1H), 8.09 (d, J = 0.9 Hz, 1H), 7.14 (dd, J = 7.0, 4.2 Hz, 1H), 6.75 (s, 1H), 6.46 (s, 1H), 6.25-6.32 (m, 1H), 4.66 (s, 2H), 4.07- 4.15 (m, 2H), 3.62 (s, 3H), 3.33-3.41 (m, 2H) | (Method 3): Rt = 3.85 min, ES⁺ m/z 517.2 [M + H]⁺ |
| A7 | 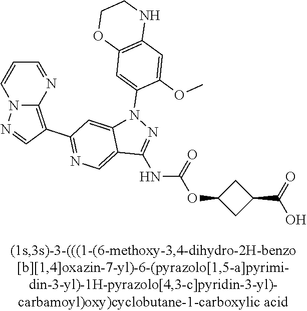 [SMILES: COc1cc2c(cc1-n1nc(NC(=O)O[C@H]3C[C@@H](C(=O)O)C3)c3cnc(-c4cnn5cccnc45)cc31)OCCN2] | Example 40 | (500 MHz, DMSO-d₆) δ: 12.11-12.40 (m, 1H), 10.56 (m, 1H), 9.21-9.29 (m, 2H), 8.91 (s, 1H), 8.73 (d, J = 4.0 Hz, 1H), 8.10 (s, 1H), 7.17 (dd, J = 6.7 Hz, J = 4.0 Hz, 1H), 6.75 (s, 1H), 6.47 (s, 1H), 6.32 (br s, 1H), 4.94-5.04 (m, 1H), 4.08-4.16 (m, 2H), 3.63 (s, 3H), 3.35-3.42 (m, 2H, over- lapping with HDO), 2.72-2.81 (m, 1H), 2.57-2.67 (m, 2H, overlapping with DMSO), 2.20-2.29 (m, 2H) | (Method 3): Rt = 4.17 min, ES⁺ m/z 557.2 [M + H]⁺ |
| A8 | 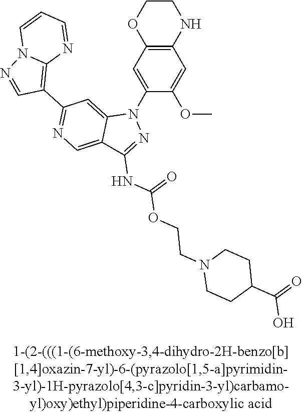 [SMILES: COc1cc2c(cc1-n1nc(NC(=O)OCCN3CCC(C(=O)O)CC3)c3cnc(-c4cnn5cccnc45)cc31)OCCN2] | Example 41 | (600 MHz, DMSO-d₆) δ: 10.44 (bs, 1H), 9.25 (d, J = 1.1, 1H), 9.21 (dd, J = 7.0, 1.6 Hz, 1H), 8.88 (s, 1H), 8.71 (dd, J = 4.2, 1.9 Hz, 1H), 8.09 (d, J = 0.9 Hz, 1H), 7.14 (dd, J = 6.9, 4.1 Hz, 1H), 6.74 (s, 1H), 6.47 (s, 1H), 6.29 (bs, 1H), 4.25 (t, J = 5.9 Hz, 2H), 4.12 (t, J = 4.8 Hz, 2H), 3.63 (s, 3H), 3.36 (m, 2H, overlapped with HDO), 2.83-2.88 (m, 2H), 2.57 (t, J = 5.4 Hz, 2H), 1.96-2.02 (m, 2H), 1.85-1.91 (m, 1H), 1.69-1.76 (m, 2H), 1.49-1.57 (m, 2H) | (Method 3): Rt = 3.45 min, ES⁺ m/z 614.3 [M + H]⁺ |

### Example A9 (or Example 46-Intermediate 1)

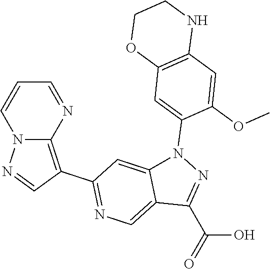 [SMILES: COc1cc2c(cc1-n1nc(C(=O)O)c3cnc(-c4cnn5cccnc45)cc31)OCCN2]

### 1-(6-Methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylic acid (Example A9)

See procedure for the preparation of Example 46-intermediate 1.

LCMS (Method 1), Rt=3.79 min, ES⁺ m/z 444.1 [M+H]⁺

¹H-NMR (500 MHz, DMSO-d₆) δ: 9.47 (s, 1H), 9.19 (dd, J=7.0, 1.6, 1H), 8.88 (s, 1H), 8.71 (dd, J=3.9, 1.5 Hz, 1H), 8.15 (d, J=0.6 Hz, 1H), 7.13 (dd, J=6.9, 3.9 Hz, 1H), 6.78 (s, 1H), 6.48 (s, 1H), 6.27 (bs, 1H), 4.14 (t, J=4.2, 2.0 Hz), 3.63 (s, 3H), 3.25-3.41 (m, 2H)

### Example A10

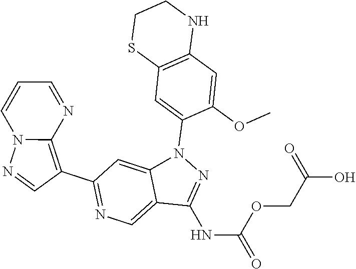 [SMILES: COc1cc2c(cc1-n1nc(NC(=O)OCC(=O)O)c3cnc(-c4cnn5cccnc45)cc31)SCCN2]

### 2-(((1-(6-Methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamoyl)oxy)acetic acid (Example A10)

The title compound was obtained as secondary product during the preparation of example 3.

LCMS (Method 1), Rt=4.20 min, ES⁺ m/z 533.1 [M+H]⁺

¹H-NMR (500 MHz, DMSO-d₆) δ: 13.11 (br s, 1H); 10.76 (br s, 1H); 9.20-9.29 (m, 2H); 8.90 (s, 1H); 8.74 (dd, J=4.0, 1.5 Hz, 1H); 8.10 (s, 1H); 7.17 (dd, J=6.9, 4.1 Hz, 1H); 6.96 (s, 1H); 6.56-6.65 (m, 1H); 6.43 (s, 1H); 4.70 (s, 2H); 3.64 (s, 3H); 3.54-3.61 (m, 2H); 2.95-3.02 (m, 2H).

### Examples A11

The following examples were prepared in a similar manner to example A1 from the indicated starting material.

| | | Starting | | |
|---|---|---|---|---|
| Example | Structure/name | Material | ¹H-NMR | LCMS |
| A11 | 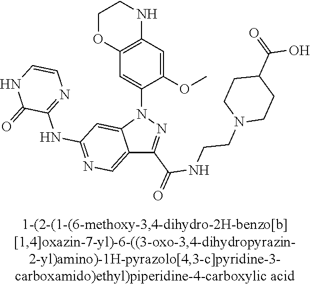 [SMILES: COc1cc2c(cc1-n1nc(C(=O)NCCN3CCC(C(=O)O)CC3)c3cnc(Nc4ncc[nH]c4=O)cc31)OCCN2] | Example 58 | (500 MHz, DMSO-d₆) δ: 9.04 (s, 1H), 9.00 (bs, 1H), 8.27 (t, J = 5.7 Hz, 1H), 8.11 (s, 1H), 7.05 (d, J = 3.0 Hz, 1H), 6.78 (s, 1H), 1.41 (s J = 3.0 Hz, 1H), 6.46 (s, 1H), 6.31 (bs, 1H), 4.12 (m, 2H), 3.62 (s, 3H), 3.45 (m, 2H), overlapped with HDO), 3.38 (m, 2H, overlapped with HDO), 2.78-2.82 (m, 2H), 2.42 (t, J = 6.9 Hz, 2H), 1.88-1.93 (m, 2H), 1.70-1.75 (m, 1H), 1.64-1.69 (m, 2H), 1.42-1.50 (m, 2H). | (Method 3): Rt = 3.48 min, ES⁺ m/z 590.3 [M + H]⁺ |

### Example A12

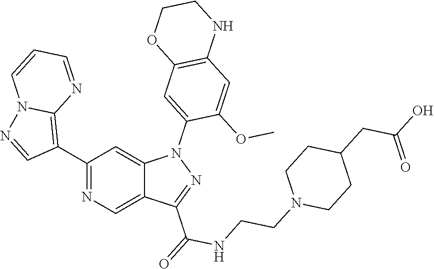 [SMILES: COc1cc2c(cc1-n1nc(C(=O)NCCN3CCC(CC(=O)O)CC3)c3cnc(-c4cnn5cccnc45)cc31)OCCN2]

### 2-(1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidin-4-yl)acetic acid (Example A12)

Title compound was prepared on a similar manner to step 3 of Example 47 starting from Example 48-Intermediate 1.

LCMS (Method 3), Rt=3.77 min, ES⁺ m/z 612.2 [M+H]⁺

¹H-NMR (500 MHz, DMSO-d₆) δ: 12.03 (br s, 1H); 9.46 (d, J=1.2 Hz, 1H); 9.23 (dd, J=7.0, 1.5 Hz, 1H); 8.91 (s, 1H); 8.74 (dd, J=4.1, 1.7 Hz, 1H); 8.42 (t, J=5.8 Hz, 1H); 8.24 (d, J=0.9 Hz, 1H); 7.17 (dd, J=6.9, 4.1 Hz, 1H); 6.89 (s, 1H); 6.49 (s, 1H); 6.37-6.40 (m, 1H); 4.14 (t, J=4.1 Hz, 2H); 3.63 (s, 3H); 3.41-3.46 (m, 2H); 3.37-3.41 (m, 2H); 2.86-2.92 (m, 2H); 2.44-2.52 (m, 2H, overlapped with DMSO); 2.12 (d, J=6.4 Hz, 2H); 1.95 (br t, J=11.0 Hz, 2H); 1.59-1.67 (m, 3H); 1.13-1.24 (m, 2H).

### PHARMACOLOGICAL ACTIVITY OF THE COMPOUNDS OF THE INVENTION (1-59)

### Biochemical Potency on JAK1, JAK2, JAK3 and Tyk2

### Assay Principle

The objective of this study was to assess the capability of compounds to inhibit all 4 JAK isoforms activity in a cell-free environment. Assay for JAK 1, JAK 2, JAK 3 and TYK2 were performed by Time-resolved fluorescence resonance energy transfer (TR-FRET) technology. It consists in the interaction of two labelled binding partners detected by the energy transfer from an excited donor to an acceptor dye and measurement of light emission by the acceptor dye. LANCE Ultra kinase assay was used. In presence of JAK 1, JAK 2, JAK 3 and TYK2 kinases and ATP (corresponding to Km), the ULight peptide substrate (LANCE Ulight-JAK-1 (Tyr1023) Peptide, Perkin Elmer, TRF0121) is phosphorylated. It is then captured by Eu-anti-phospho-substrate antibody (LANCE Eu-W1024 Anti-phosphotyrosine (PT66), Perkin Elmer, AD0069), which bring the Eu-chelate donor and ULight acceptor dyes into close proximity. Upon excitation at 320 nm, the Eu-chelate transfers its energy to the ULight dye, resulting in a fluorescent light emission at 665 nm.

### Compound Testing

Serial dilutions of compounds in pure DMSO are prepared from 10 mM DMSO stock solutions. Compounds were tested in 384-well plate for 11 consecutive 5-fold dilutions starting from 20 μM highest concentration (20 μM-2 μM). 200 nL of compound were transferred from mother plate to test plate by using Mosquito (TTP labtech). Assay was performed in 384-well Perkin Elmer test plate in 20 μL assay volume (kinase reaction) and 40 μL total volume (stopping reagent and antibody detection reagents). In 10 μL of substrate solution (peptide+ATP) 30/50/20/10 nM of peptide and 20/0.7/0.2/12 μM of ATP were added for JAK 1, JAK 2, JAK 3 and TYK2 respectively. 10 μL of enzyme solution was added to kinase reaction at these concentrations: 0.15/0.083/0.025/0.144 ng/μL of JAK 1, JAK 2, JAK 3 and TYK2 respectively. After shaking and 1.5 h of incubation at r.t., 20 μL of Stop (10 μL EDTA) and Detection mixture (10 μL Europium-anti-phospho antibody, final: 0.5 nM) were added. Reading was performed after 1 h of incubation on a En Vision 2104 reader (Perkin Elmer).

Calculation of IC50 data, curves and QC analysis was performed by using Excel tool and GraphPadPrism software, v9. Briefly, individual concentration-effect curves are generated by plotting the logarithm of the tested concentration of tested compounds (X) vs. corresponding percent inhibition values (Y) using least squares (ordinary) fit. Best fit IC50 values are calculated using Log (inhibitor) vs. normalized response—Variable slope equation, where Y=100/(1+10\hat{ }((LogIC50−X)*HillSlope)). QC criteria parameters (Z′, S:B, R₂, HillSlope) were checked for every IC50 curve. Calculation of IC50 data, curves and QC analysis were made using Excel tools and GraphPadPrism software. QC criteria parameters: Z′≥0.5, Hill Slope range 0.5 to 5, S:B>2.

Compounds according to the invention showed values in terms of pIC50 higher than 6 with respect to their inhibitory activity on all JAK isoforms corresponding to ≤1 μM in terms of inhibitory concentration. Most compounds preferably showed values higher than 7.3, even more preferably higher than 8.3, at least with respect to their inhibitory activity on JAK1; corresponding to ≤50 nM, even more preferably ≤5 nM, in terms of inhibitory concentration.

Data for compounds 1-46 are reported in the table hereinbelow

| Example No | JAK1 | JAK2 | JAK3 | TYK2 |
|---|---|---|---|---|
| 1 | +++ | +++ | +++ | +++ |
| 2 | +++ | +++ | +++ | ++ |
| 3 | +++ | +++ | +++ | ++ |
| 4 | +++ | +++ | +++ | +++ |
| 5 | +++ | +++ | +++ | ++ |
| 6 | ++ | +++ | +++ | + |
| 7 | +++ | +++ | +++ | +++ |
| 8 | +++ | +++ | +++ | +++ |
| 9 | +++ | +++ | +++ | ++ |
| 10 | +++ | +++ | +++ | ++ |
| 11 | +++ | +++ | +++ | ++ |
| 12 | +++ | +++ | +++ | ++ |
| 13 | +++ | +++ | +++ | ++ |
| 14 | +++ | +++ | +++ | ++ |
| 15 | +++ | +++ | +++ | ++ |
| 16 | +++ | +++ | +++ | ++ |
| 17 | +++ | +++ | +++ | ++ |
| 18 | +++ | +++ | +++ | ++ |
| 19 | +++ | +++ | +++ | ++ |
| 20 | +++ | +++ | +++ | ++ |
| 21 | +++ | +++ | +++ | ++ |
| 22 | ++ | +++ | +++ | ++ |
| 23 | +++ | +++ | +++ | ++ |
| 24 | +++ | +++ | +++ | ++ |
| 25 | +++ | +++ | +++ | ++ |
| 26 | ++ | +++ | +++ | ++ |
| 27 | +++ | +++ | +++ | ++ |
| 28 | +++ | +++ | +++ | ++ |
| 29 | +++ | +++ | +++ | ++ |
| 30 | +++ | +++ | +++ | ++ |
| 31 | +++ | +++ | +++ | ++ |
| 32 | +++ | +++ | +++ | ++ |
| 33 | +++ | +++ | +++ | ++ |
| 34 | +++ | +++ | +++ | ++ |
| 35 | +++ | +++ | +++ | ++ |
| 36 | +++ | ++ | +++ | ++ |
| 37 | ++ | +++ | +++ | ++ |
| 38 | ++ | ++ | +++ | ++ |
| 39 | +++ | +++ | +++ | ++ |
| 40 | +++ | +++ | +++ | ++ |
| 41 | +++ | +++ | +++ | ++ |
| 42 | +++ | +++ | +++ | ++ |
| 43 | +++ | +++ | +++ | ++ |
| 44 | +++ | +++ | +++ | ++ |
| 45 | +++ | +++ | +++ | ++ |
| 46 | +++ | +++ | +++ | ++ |
| 47 | +++ | +++ | +++ | ++ |
| 48 | +++ | +++ | +++ | ++ |
| 49 | +++ | +++ | +++ | ++ |
| 50 | +++ | +++ | +++ | ++ |
| 51 | +++ | +++ | +++ | ++ |
| 52 | +++ | +++ | +++ | ++ |
| 53 | +++ | +++ | +++ | ++ |
| 54 | +++ | +++ | +++ | ++ |
| 55 | +++ | +++ | +++ | ++ |
| 56 | +++ | +++ | +++ | ++ |
| 57 | +++ | +++ | +++ | ++ |
| 58 | +++ | +++ | +++ | ++ |
| 59 | +++ | +++ | +++ | ++ |

The compounds are classified in the table above in term of potency with respect to their inhibitory activity on JAKI, JAK2, JAK3 and Tyk2 isoforms according to the following classification criterion:
+++: pIC₅₀≥8.3
++: 8.3>pIC₅₀≥7.3
+: pIC₅₀<7.3
   Inhibition of IL-13 Induced pSTAT6 in BEAS

BEAS-2B human cell line was seeded (100.000 cells/well) and incubated for 48 h at 37° C., 5% CO2, 95% humidity. Compounds were added and incubated 30 min followed by IL-13 as trigger. After 30 min incubation, cells were lysed and pSTAT6 determined by Fastscan phospho-stat6 (Tyr641) sandwich ELISA kit (cell signaling). Inhibitors were tested at 11 consecutive 5-fold dilutions starting from 10 μM (10 μM-40 μM) in duplicate. Calculation of IC50 data, curves and QC analysis were made using Excel tools and GraphPadPrism software. QC criteria parameters: Z′≥0.35, Hill Slope range 0.5 to 5, S:B>2.

Compounds according to the invention showed measurable values in terms of pIC50 (BEAS) higher than 7.3.

Predicted acid metabolites showed measurable values in terms of pIC50 (BEAS) higher than 5.

| | | | pIC₅₀ | Fold difference |
|---|---|---|---|---|
| | | | (BEAS) | parent vs |
| | | Predicted | predicted | predicted |
| | pIC₅₀ | acid | acid | acid |
| Example No | (BEAS) | metabolite | metabolite | metabolite |
| 1 | §§§§ | A2 | §§ | kk |
| 2 | §§§ | | | |
| 3 | §§§§ | A10 | § | kkkk |
| 4 | §§§§ | A5 | §§ | kk |
| 5 | §§§§ | A1 | § | kkkk |
| 6 | §§§ | | | |
| 7 | §§§§ | | | |
| 8 | §§§ | | | |
| 9 | §§§§ | A1 | § | kkkk |
| 10 | §§§§ | A1 | § | kkkk |
| 11 | §§§§ | A1 | § | kkkk |
| 12 | §§§§ | A1 | § | kkkk |
| 13 | §§§§ | A1 | § | kkkk |
| 14 | §§§§ | A1 | § | kkkk |
| 15 | §§§§ | A1 | § | kkkk |
| 16 | §§§§ | A1 | § | kkkk |
| 17 | §§§§ | A1 | § | kkkk |
| 18 | §§§§ | A1 | § | kkkk |
| 19 | §§§§ | A1 | § | kkkk |
| 20 | §§§ | A1 | § | kkk |
| 21 | §§§§ | A1 | § | kkkk |
| 22 | §§§ | A1 | § | kkk |
| 23 | §§§ | A1 | § | kkk |
| 24 | §§§ | A1 | § | kkk |
| 25 | §§§§ | A1 | § | kkkk |
| 26 | §§§ | | | |
| 27 | §§§ | | | |
| 28 | §§§ | | | |
| 29 | §§§§ | A4 | § | kkk |
| 30 | §§§ | A3 | § | kkkk |
| 31 | §§§ | | | |
| 32 | §§§ | | | |
| 33 | §§§ | | | |
| 34 | §§§ | | | |
| 35 | §§§§ | A12 | § | kkkk |
| 36 | §§§ | | | |
| 37 | §§§ | | | |
| 38 | §§§§ | | | |
| 39 | §§§ | A6 | § | kkkk |
| 40 | §§§ | A7 | § | kk |
| 41 | §§§§ | A8 | § | kkk |
| 42 | §§§ | A1 | § | kkk |
| 43 | §§§ | A4 | § | kkk |
| 44 | §§§ | A3 | § | kkkk |
| 45 | §§§§ | A1 | § | kkkk |
| 46 | §§§ | A9 | § | kkk |
| 47 | §§§§ | A1 | § | kkkk |
| 48 | §§§ | A12 | § | kkkk |
| 49 | §§§§ | A4 | § | kkk |
| 50 | §§§§ | A1 | § | kkkk |
| 51 | §§§ | A1 | § | kkk |
| 52 | §§§ | A12 | § | kkk |
| 53 | §§§§ | A1 | § | kkkk |
| 54 | §§§§ | A1 | § | kkkk |
| 55 | §§§ | A4 | § | kk |
| 56 | §§§ | A3 | § | kk |
| 57 | §§§ | A1 | § | kkk |
| 58 | §§§ | A11 | § | kkkk |
| 59 | §§§§ | A1 | § | kkkk |

The compounds are classified in the table above in term of potency with respect to their functional activity in BEAS according to the following classification criterion:
§ § § §: pIC₅₀≥8.3
§ § §: 8.3>pIC₅₀≥7.3
§ §: 7.3>pIC₅₀≥6.3
§: pIC₅₀<6.3
and in terms of fold difference (ratio) of IC₅₀ of corresponding predicted acid metabolite vs parent ester (ratio=IC₅₀ predicted acid metabolite/IC₅₀ ester)
kkkk: ratio≥200
kkk: 200>ratio≥100
kk: 100>ratio≥10
k: ratio<10

Where a numerical limit or range is stated herein, the endpoints are included. Also, all values and subranges within a numerical limit or range are specifically included when not explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

1: A compound of formula I-1 or I-2 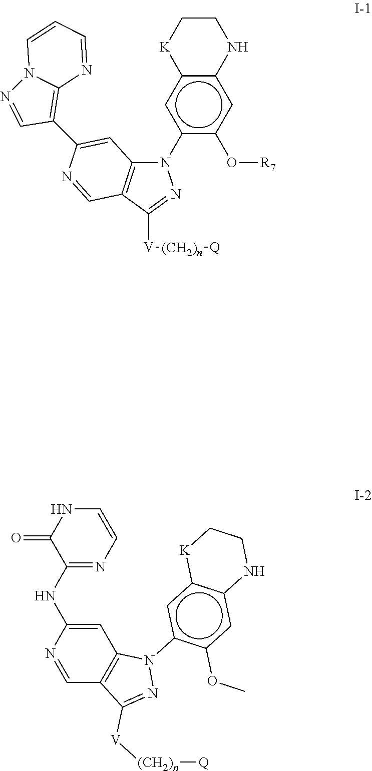
wherein the substituent group 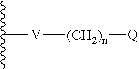
comprises at least one ester or thioester moiety;
V is a divalent group selected from the group consisting of C(O)O, C(O)N(R₆), and N(R₆)C(O)O;
Q is selected from the group consisting of (C₁-C₆)alkoxycarbonyl, —(CH₂)ₘNR₄R₅, (C₃-C₈)cycloalkyl, and (C₃-C₆)heterocycloalkyl; wherein said (C₃-C₈)cycloalkyl and (C₃-C₆)heterocycloalkyl are optionally substituted by one or more substituent groups selected from the group consisting of (C₁-C₆)alkoxycarbonyl(CH₂)ₘ, (C₃-C₈)cycloalkyl-oxycarbonyl, NC—(C₁-C₆)alkoxycarbonyl, (C₁-C₆)haloalkyl-oxycarbonyl, (C₃-C₈)heterocycloalkyl-oxycarbonyl, (C₁-C₆)alkyl (C₃-C₈)heterocycloalkyl-oxycarbonyl, (C₃-C₈)heterocycloalkyl-(C₁-C₆)alkoxycarbonyl, (C₁-C₆)alkyl-(C₃-C₅)heterocycloalkyl-(C₁-C₆)alkoxycarbonyl, (C₁-C₆) hydroxyalkyl-oxycarbonyl, (C₁-C₆) aminoalkyl-oxycarbonyl, (C₁-C₆)alkoxy-(C₁-C₆)alkyl-oxycarbonyl, (C₁-C₆)alkylthiocarbonyl, NC—(C₃-C₈)cycloalkyl (C₁-C₆)alkoxycarbonyl, NC—(C₃-C₈)cycloalkyl-oxycarbonyl, (C₁-C₆)haloalkyl-(C₃-C₈)heterocycloalkyl-oxycarbonyl, halo-((C₁-C₆)alkyl(C₃-C₈)heterocycloalkyl)-oxycarbonyl, (C₁-C₆)haloalkyl-(C₃-C₈)heterocycloalkyl-(C₁-C₆)alkoxycarbonyl, (C₁-C₆)haloalkyl-(C₃-C₈)heterocycloalkyl-oxycarbonyl-(C₁-C₆)alkyl, (C₁-C₆) hydroxyalkyl-oxycarbonyl-(C₁-C₆)alkyl, (C₁-C₁₀)alkyl, and halogen;
K is selected from the group consisting of O, CH₂, and S;
n and m are in each occurrence independently 0 or an integer selected from the group consisting of 1, 2, 3 and 4;
R₄ and R₅, the same or different, are selected from the group consisting of
H,
(C₁-C₆)alkyl,
(C₁-C₆)haloalkyl,
(C₃-C₆)heterocycloalkyl,
(C₁-C₆)alkyl-(C₃-C₈)heterocycloalkyl,
(C₁-C₆)alkoxycarbonyl-methyl,
(C₁-C₆)alkoxycarbonyl-phenyl-methyl, and
(C₁-C₆)alkoxycarbonyl-(C₁-C₆)alkyl-phenyl-methyl;
R₆ is in each occurrence independently selected from the group consisting of H, (C₁-C₆)alkyl, and (C₁-C₆) hydroxyalkyl;
a single enantiomer, a diastereoisomer, a mixture thereof,
or a pharmaceutically acceptable salt or solvate thereof.

2: The compound, single enantiomer, diastereoisomer, mixture thereof, or pharmaceutically acceptable salt or solvate thereof, according to claim 1, wherein K is O and the compound is represented by formula (Ia) 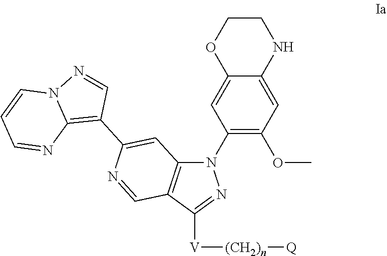
wherein
V is C(O)N(R₆),
Q is (C₃-C₆)heterocycloalkyl substituted by at least one group selected from the group consisting of (C₁-C₆)alkoxycarbonyl, (C₃-C₈)cycloalkyl-oxycarbonyl, NC—(C₁-C₆) alkoxycarbonyl, (C₁-C₆)haloalkyl-oxycarbonyl, (C₁-C₆)alkyl (C₃-C₈)heterocycloalkyl-oxycarbonyl, (C₁-C₆)hydroxyalkyl-oxycarbonyl and (C₁-C₆)alkoxy (C₁-C₆)alkyl-oxycarbonyl and (C₁-C₆)alkylthiocarbonyl;
n is in each occurrence independently 0 or an integer selected from the group consisting of 1, 2, 3 and 4; and
R₆ is in each occurrence independently selected from the group consisting of H, (C₁-C₆)alkyl and (C₁-C₆) hydroxyalkyl.

3: The compound, single enantiomer, diastereoisomer, mixture thereof, or pharmaceutically acceptable salt or solvate thereof, according to claim 2, wherein the compound is selected from the group consisting of
ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
isopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
cyclopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-cyano-2-methylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2,2,2-trifluoroethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
1-methylazetidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
3-hydroxy-2,2-dimethylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-hydroxy-2-methylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
1-methoxy-2-methylpropan-2-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(R)-1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate; and
S-ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carbothioate.

4: The compound, single enantiomer, diastereoisomer, mixture thereof, or pharmaceutically acceptable salt or solvate thereof, according to claim 1, wherein
V is C(O)O;
Q is selected from the group consisting of (C₁-C₆)alkoxycarbonyl, —(CH₂)ₘNR₄R₅, and (C₃-C₆)heterocycloalkyl optionally substituted by one or more groups selected from the group consisting of (C₁-C₁₀)alkyl and halogen;
n and m are in each occurrence independently 0 or an integer selected from the group consisting of 1, 2, 3 and 4;
R₄ and R₅, the same or different, are selected from the group consisting of H and (C₁-C₆)alkyl.

5: The compound, single enantiomer, diastereoisomer, mixture thereof, or pharmaceutically acceptable salt or solvate thereof, according to claim 1, wherein K is S and the compound is represented by formula (Ib) 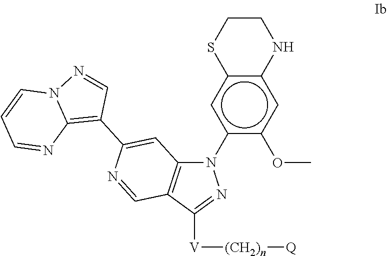
wherein
V is N(R₆)C(O)O;
Q is (C₁-C₆)alkoxycarbonyl;
n is an integer selected from the group consisting of 1, 2, 3 and 4; and
R₆ is in each occurrence independently selected from the group consisting of H and (C₁-C₆)alkyl.

6: The compound, single enantiomer, diastereoisomer, mixture thereof, or pharmaceutically acceptable salt or solvate thereof, according to claim 1, wherein the compound is selected from the the group consisting of
ethyl 1-(2-(1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridine-3-carboxamido)ethyl)piperidine-4-carboxylate;
ethyl (1s,3s)-3-(((1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamoyl)oxy)cyclobutane-1-carboxylate;
ethyl 2-(((1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamoyl)oxy) acetate;
ethyl 1-(2-(((1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridin-3-yl)carbamoyl)oxy)ethyl)piperidine-4-carboxylate;
1-methoxy-2-methylpropan-2-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
propyl N-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)-N-methylglycinate;
3-(dimethylamino) propyl 1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridine-3-carboxylate;
3-(4-methylpiperazin-1-yl)propyl 1-(6-methoxy-3,4-dihydro-2H-benzo[b][1,4]thiazin-7-yl)-6-(pyrazolo[1,5-a]pyrimidin-3-yl)-1H-pyrazolo[4,3-c]pyridine-3-carboxylate;
ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
sec-butyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
cyclopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
cyclopentyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2,2,2-trifluoroethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-hydroxy-2-methylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
3-hydroxy-2,2-dimethylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-methoxyethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
tetrahydro-2H-pyran-4-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(tetrahydro-2H-pyran-4-yl)methyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(4-methylmorpholin-2-yl)methyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-(dimethylamino)-2-methylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
1-methylazetidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(R)-1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(S)-1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-cyano-2-methylpropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
1-methylpiperidin-4-yl 1-(3-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido) propyl)piperidine-4-carboxylate;
ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-3-carboxylate;
ethyl 4-fluoro-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
methyl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
methyl (S)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl) azetidine-3-carboxylate;
ethyl (3aR,5r,6aS)-2-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl) octahydrocyclopenta[c]pyrrole-5-carboxylate;
ethyl (1R,5S,6r)-3-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)-3-azabicyclo[3.1.0]hexane-6-carboxylate;
ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)-4-methylpiperidine-4-carboxylate;
ethyl 2-(1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidin-4-yl)acetate;
ethyl 4-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)-1-methylpiperazine-2-carboxylate;
methyl 2-(3-(((2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)(methyl)amino)methyl)phenyl) acetate;
ethyl 1-(2-(1-(7-methoxy-1,2,3,4-tetrahydroquinolin-6-yl)-6-(pyrazolo[1,5-a]pyrimidin-3-yl)-1H-pyrazolo[4,3-c]pyridine-3-carboxamido)ethyl)piperidine-4-carboxylate;
ethyl 2-(((1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamoyl)oxy) acetate;
ethyl (1s,3s)-3-(((1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl) carbamoyl)oxy)cyclobutane-1-carboxylate;
ethyl 1-(2-(((1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridin-3-yl)carbamoyl)oxy)ethyl)piperidine-4-carboxylate;
1-methylpiperidin-4-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
ethyl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
ethyl (S)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
isopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(4-methylmorpholin-2-yl)methyl 1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxylate;
3-cyanocyclobutyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
2-hydroxy-2-methylpropyl 2-(1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidin-4-yl)acetate;
2-cyano-2-methylpropyl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
(3S,4S)-4-fluoro-1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(3R*,4R*)-4-fluoro-1-methylpyrrolidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(R)-1-(2-fluoroethyl)pyrrolidin-3-yl 2-(1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidin-4-yl)acetate;
(1-cyanocyclopropyl)methyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
3-cyanopropyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
(R)-1-(2-fluoroethyl)pyrrolidin-3-yl (R)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
(R)-1-methylpyrrolidin-3-yl(S)-1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)pyrrolidine-3-carboxylate;
1-isopropylazetidin-3-yl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carboxylate;
ethyl 1-(2-(1-(6-methoxy-3,4-dihydro-2H-benzo[b][1,4]oxazin-7-yl)-6-((3-oxo-3,4-dihydropyrazin-2-yl)amino)-1H-pyrazolo[4,3-c]pyridine-3-carboxamido)ethyl)piperidine-4-carboxylate; and
S-ethyl 1-(2-(1-(6-methoxy-3,4-dihydro-2H-benzo[b][1,4]oxazin-7-yl)-6-(pyrazolo[1,5-a]pyrimidin-3-yl)-1H-pyrazolo[4,3-c]pyridine-3-carboxamido)ethyl)piperidine-4-carbothioate.

7: A pharmaceutical composition, comprising the compound, single enantiomer, diastereoisomer, mixture thereof, or pharmaceutically acceptable salt or solvate thereof, according to claim 1, in admixture with at least one pharmaceutically acceptable carrier or excipient.

8: The pharmaceutical composition according to claim 7, which is formulated as inhalable powders, propellant-containing metering aerosols, or propellant-free inhalable formulations, to be administered by inhalation.

9: A device comprising the pharmaceutical composition according to claim 8, which is a single- or multi-dose dry powder inhaler, a metered dose inhaler, or a soft mist nebulizer.

10: A medicament, comprising the compound, single enantiomer, diastereoisomer, mixture thereof, or pharmaceutically acceptable salt or solvate thereof, according to claim 1.

11: A method for preventing and/or treating a pulmonary disease, the method comprising:
administering the medicament according to claim 10 to a subject in need thereof,
wherein the pulmonary disease is selected from the group consisting of asthma, chronic obstructive pulmonary disease COPD, idiopathic pulmonary fibrosis (IPF), acute lung injury and acute respiratory distress syndrome (ARDS).

12: A composition, comprising:
the compound, single enantiomer, diastereoisomer, mixture thereof, or pharmaceutically acceptable salt or solvate thereof, according to claim 1, and
one or more active ingredients for treating a respiratory disorder selected from the the group consisting of a beta2-agonist, an antimuscarinic agent, a corticosteroid, a mitogen-activated kinases (P38 MAP kinases) inhibitor, a PI3K (phosphoinositide 3-kinases) inhibitor, a nuclear factor kappa-B kinase subunit beta inhibitor (IKK2), a Rho kinase inhibitor (ROCKi), a human neutrophil elastase (HNE) inhibitor, a phosphodiesterase 4 (PDE4) inhibitor, a leukotriene modulator, a non-steroidal anti-inflammatory agent (NSAIDs), and a mucus regulator.
